(19) United States
(12) Patent Application Publication
(10) Pub. No.: US 2025/0375690 A1
(43) Pub. Date: Dec. 11, 2025

(54) WORKOUT SAFETY USER INTERFACES

(71) Applicant: Apple Inc., Cupertino, CA (US)

(72) Inventor: Edward CHAO, Palo Alto, CA (US)

(21) Appl. No.: 19/187,477

(22) Filed: Apr. 23, 2025

Related U.S. Application Data
(60) Provisional application No. 63/657,934, filed on Jun. 9, 2024.

(51) Int. Cl. A63B 71/00; A63B 24/00; A63B 71/06; G16H 20/30; H04M 1/72418

(52) U.S. Cl. CPC A63B 71/0054; A63B 24/0087; A63B 71/0622; G16H 20/30; H04M 1/72418; A63B 2071/0675

(57) ABSTRACT

The present disclosure generally relates to techniques for managing workout-based notifications.

### CROSS-REFERENCE TO RELATED APPLICATIONS

This application claims priority to U.S. Provisional Patent Application Ser. No. 63/657,934, entitled “WORKOUT SAFETY USER INTERFACES,” filed on Jun. 9, 2024, the contents of which are hereby incorporated by reference in their entirety.

### FIELD

The present disclosure relates generally to computer user interfaces, and more specifically to techniques for managing workout-based notifications.

### BACKGROUND

Devices detect users performing physical activities and update user interfaces according to the physical activities detected. This allows users to review the physical activities they have performed.

### BRIEF SUMMARY

Some techniques for managing workout-based notifications using electronic devices, however, are generally cumbersome and inefficient. For example, some existing techniques use a complex and time-consuming user interface, which may include multiple key presses or keystrokes. Existing techniques require more time than necessary, wasting user time and device energy. This latter consideration is particularly important in battery-operated devices.

Accordingly, the present technique provides electronic devices with faster, more efficient methods and interfaces for managing workout-based notifications. Such methods and interfaces optionally complement or replace other methods for managing workout-based notifications. Such methods and interfaces reduce the cognitive burden on a user and produce a more efficient human-machine interface. For battery-operated computing devices, such methods and interfaces conserve power and increase the time between battery charges.

In some embodiments, a method is disclosed. The method is performed at a computer system that is in communication with one or more display generation components and one or more input devices, and comprises: detecting, via the one or more input devices, a first user input corresponding to a user request to initiate a workout session; in response to detecting the first user input, initiating a workout session; while the workout session is active, detecting, via the one or more input devices, one or more user inputs corresponding to a user request to activate a workout-based check-in session with an external computer system separate from the computer system, including selection of a displayed check-in option; in response to detecting the one or more user inputs corresponding to the user request to activate the workout-based check-in session with an external computer system, initiating a process for activating a workout-based check-in session with a first external computer system separate from the computer system, wherein, while the workout-based check-in session is active, the computer system automatically transmits notifications to the first external computer system based on satisfaction of notification criteria pertaining to the computer system; and while the workout-based check-in session with the first external computer system is active: in accordance with a determination that notification criteria are satisfied, transmitting check-in notification information to the first external computer system, wherein the check-in notification information causes the first external computer system to output information corresponding to the computer system.

In some embodiments, a non-transitory computer-readable storage medium is disclosed. The non-transitory computer-readable storage medium stores one or more programs configured to be executed by one or more processors of a computer system that is in communication with one or more display generation components and one or more input devices, and the one or more programs include instructions for: detecting, via the one or more input devices, a first user input corresponding to a user request to initiate a workout session; in response to detecting the first user input, initiating a workout session; while the workout session is active, detecting, via the one or more input devices, one or more user inputs corresponding to a user request to activate a workout-based check-in session with an external computer system separate from the computer system, including selection of a displayed check-in option; in response to detecting the one or more user inputs corresponding to the user request to activate the workout-based check-in session with an external computer system, initiating a process for activating a workout-based check-in session with a first external computer system separate from the computer system, wherein, while the workout-based check-in session is active, the computer system automatically transmits notifications to the first external computer system based on satisfaction of notification criteria pertaining to the computer system; and while the workout-based check-in session with the first external computer system is active: in accordance with a determination that notification criteria are satisfied, transmitting check-in notification information to the first external computer system, wherein the check-in notification information causes the first external computer system to output information corresponding to the computer system.

In some embodiments, a transitory computer-readable storage medium is disclosed. The transitory computer-readable storage medium stores one or more programs configured to be executed by one or more processors of a computer system that is in communication with one or more display generation components and one or more input devices, and the one or more programs include instructions for: detecting, via the one or more input devices, a first user input corresponding to a user request to initiate a workout session; in response to detecting the first user input, initiating a workout session; while the workout session is active, detecting, via the one or more input devices, one or more user inputs corresponding to a user request to activate a workout-based check-in session with an external computer system separate from the computer system, including selection of a displayed check-in option; in response to detecting the one or more user inputs corresponding to the user request to activate the workout-based check-in session with an external computer system, initiating a process for activating a workout-based check-in session with a first external computer system separate from the computer system, wherein, while the workout-based check-in session is active, the computer system automatically transmits notifications to the first external computer system based on satisfaction of notification criteria pertaining to the computer system; and while the workout-based check-in session with the first external computer system is active: in accordance with a determination that notification criteria are satisfied, transmitting check-in notification information to the first external computer system, wherein the check-in notification information causes the first external computer system to output information corresponding to the computer system.

In some embodiments, a computer system is disclosed. The computer system is configured to communicate with one or more display generation components and one or more input devices, and comprises: one or more processors; and memory storing one or more programs configured to be executed by the one or more display generation components, the one or more programs including instructions for: detecting, via the one or more input devices, a first user input corresponding to a user request to initiate a workout session; in response to detecting the first user input, initiating a workout session; while the workout session is active, detecting, via the one or more input devices, one or more user inputs corresponding to a user request to activate a workout-based check-in session with an external computer system separate from the computer system, including selection of a displayed check-in option; in response to detecting the one or more user inputs corresponding to the user request to activate the workout-based check-in session with an external computer system, initiating a process for activating a workout-based check-in session with a first external computer system separate from the computer system, wherein, while the workout-based check-in session is active, the computer system automatically transmits notifications to the first external computer system based on satisfaction of notification criteria pertaining to the computer system; and while the workout-based check-in session with the first external computer system is active: in accordance with a determination that notification criteria are satisfied, transmitting check-in notification information to the first external computer system, wherein the check-in notification information causes the first external computer system to output information corresponding to the computer system.

In some embodiments, a computer system is disclosed. The computer system is configured to communicate with one or more display generation components and one or more input devices, and comprises: means for detecting, via the one or more input devices, a first user input corresponding to a user request to initiate a workout session; means for, in response to detecting the first user input, initiating a workout session; means for, while the workout session is active, detecting, via the one or more input devices, one or more user inputs corresponding to a user request to activate a workout-based check-in session with an external computer system separate from the computer system, including selection of a displayed check-in option; means for, in response to detecting the one or more user inputs corresponding to the user request to activate the workout-based check-in session with an external computer system, initiating a process for activating a workout-based check-in session with a first external computer system separate from the computer system, wherein, while the workout-based check-in session is active, the computer system automatically transmits notifications to the first external computer system based on satisfaction of notification criteria pertaining to the computer system; and means for, while the workout-based check-in session with the first external computer system is active: in accordance with a determination that notification criteria are satisfied, transmitting check-in notification information to the first external computer system, wherein the check-in notification information causes the first external computer system to output information corresponding to the computer system.

In some embodiments, a computer program product is disclosed. The computer program product comprises one or more programs configured to be executed by one or more processors of a computer system that is in communication with one or more display generation components and one or more input devices, the one or more programs including instructions for: detecting, via the one or more input devices, a first user input corresponding to a user request to initiate a workout session; in response to detecting the first user input, initiating a workout session; while the workout session is active, detecting, via the one or more input devices, one or more user inputs corresponding to a user request to activate a workout-based check-in session with an external computer system separate from the computer system, including selection of a displayed check-in option; in response to detecting the one or more user inputs corresponding to the user request to activate the workout-based check-in session with an external computer system, initiating a process for activating a workout-based check-in session with a first external computer system separate from the computer system, wherein, while the workout-based check-in session is active, the computer system automatically transmits notifications to the first external computer system based on satisfaction of notification criteria pertaining to the computer system; and while the workout-based check-in session with the first external computer system is active: in accordance with a determination that notification criteria are satisfied, transmitting check-in notification information to the first external computer system, wherein the check-in notification information causes the first external computer system to output information corresponding to the computer system.

In some embodiments, a method is disclosed. The method is performed at a computer system that is in communication with one or more display generation components and one or more input devices, and comprises: displaying, via the one or more display generation components, a time user interface, wherein the time user interface includes an indication of time; and while displaying the time user interface: in accordance with a determination that a workout session is active, displaying, via the one or more display generation components, a recommendation to initiate a workout-based check-in session with an external computer system, wherein, while the workout-based check-in session is active, the computer system automatically transmits notifications to the external computer system based on satisfaction of notification criteria pertaining to the computer system.

In some embodiments, a non-transitory computer-readable storage medium is disclosed. The non-transitory computer-readable storage medium stores one or more programs configured to be executed by one or more processors of a computer system that is in communication with one or more display generation components and one or more input devices, and the one or more programs include instructions for: displaying, via the one or more display generation components, a time user interface, wherein the time user interface includes an indication of time; and while displaying the time user interface: in accordance with a determination that a workout session is active, displaying, via the one or more display generation components, a recommendation to initiate a workout-based check-in session with an external computer system, wherein, while the workout-based check-in session is active, the computer system automatically transmits notifications to the external computer system based on satisfaction of notification criteria pertaining to the computer system.

In some embodiments, a transitory computer-readable storage medium is disclosed. The transitory computer-readable storage medium stores one or more programs configured to be executed by one or more processors of a computer system that is in communication with one or more display generation components and one or more input devices, and the one or more programs include instructions for: displaying, via the one or more display generation components, a time user interface, wherein the time user interface includes an indication of time; and while displaying the time user interface: in accordance with a determination that a workout session is active, displaying, via the one or more display generation components, a recommendation to initiate a workout-based check-in session with an external computer system, wherein, while the workout-based check-in session is active, the computer system automatically transmits notifications to the external computer system based on satisfaction of notification criteria pertaining to the computer system.

In some embodiments, a computer system is disclosed. The computer system is configured to communicate with one or more display generation components and one or more input devices, and comprises: one or more processors; and memory storing one or more programs configured to be executed by the one or more display generation components, the one or more programs including instructions for: displaying, via the one or more display generation components, a time user interface, wherein the time user interface includes an indication of time; and while displaying the time user interface: in accordance with a determination that a workout session is active, displaying, via the one or more display generation components, a recommendation to initiate a workout-based check-in session with an external computer system, wherein, while the workout-based check-in session is active, the computer system automatically transmits notifications to the external computer system based on satisfaction of notification criteria pertaining to the computer system.

In some embodiments, a computer system is disclosed. The computer system is configured to communicate with one or more display generation components and one or more input devices, and comprises: means for displaying, via the one or more display generation components, a time user interface, wherein the time user interface includes an indication of time; and means for, while displaying the time user interface: in accordance with a determination that a workout session is active, displaying, via the one or more display generation components, a recommendation to initiate a workout-based check-in session with an external computer system, wherein, while the workout-based check-in session is active, the computer system automatically transmits notifications to the external computer system based on satisfaction of notification criteria pertaining to the computer system.

In some embodiments, a computer program product is disclosed. The computer program product comprises one or more programs configured to be executed by one or more processors of a computer system that is in communication with one or more display generation components and one or more input devices, the one or more programs including instructions for: displaying, via the one or more display generation components, a time user interface, wherein the time user interface includes an indication of time; and while displaying the time user interface: in accordance with a determination that a workout session is active, displaying, via the one or more display generation components, a recommendation to initiate a workout-based check-in session with an external computer system, wherein, while the workout-based check-in session is active, the computer system automatically transmits notifications to the external computer system based on satisfaction of notification criteria pertaining to the computer system.

Executable instructions for performing these functions are, optionally, included in a non-transitory computer-readable storage medium or other computer program product configured for execution by one or more processors. Executable instructions for performing these functions are, optionally, included in a transitory computer-readable storage medium or other computer program product configured for execution by one or more processors.

Thus, devices are provided with faster, more efficient methods and interfaces for managing workout-based notifications, thereby increasing the effectiveness, efficiency, and user satisfaction with such devices. Such methods and interfaces may complement or replace other methods for managing workout-based notifications.

### DESCRIPTION OF THE FIGURES

For a better understanding of the various described embodiments, reference should be made to the Description of Embodiments below, in conjunction with the following drawings in which like reference numerals refer to corresponding parts throughout the figures.

FIG. 1A is a block diagram illustrating a portable multifunction device with a touch-sensitive display in accordance with some embodiments.

FIG. 1B is a block diagram illustrating exemplary components for event handling in accordance with some embodiments.

FIG. 2 illustrates a portable multifunction device having a touch screen in accordance with some embodiments.

FIG. 3A is a block diagram of an exemplary multifunction device with a display and a touch-sensitive surface in accordance with some embodiments.

FIGS. 3B-3G illustrate the use of Application Programming Interfaces (A Pls) to perform operations.

FIG. 4A illustrates an exemplary user interface for a menu of applications on a portable multifunction device in accordance with some embodiments.

FIG. 4B illustrates an exemplary user interface for a multifunction device with a touch-sensitive surface that is separate from the display in accordance with some embodiments.

FIG. 5A illustrates a personal electronic device in accordance with some embodiments.

FIG. 5B is a block diagram illustrating a personal electronic device in accordance with some embodiments.

FIGS. 6A-6A C illustrate exemplary user interfaces for managing workout-based notifications, in accordance with some embodiments.

FIG. 7 illustrates a flow diagram depicting a method for managing workout-based notifications, in accordance with some embodiments.

FIG. 8 illustrates a flow diagram depicting a method for managing workout-based notifications, in accordance with some embodiments.

### DESCRIPTION OF EMBODIMENTS

The following description sets forth exemplary methods, parameters, and the like. It should be recognized, however, that such description is not intended as a limitation on the scope of the present disclosure but is instead provided as a description of exemplary embodiments.

There is a need for electronic devices that provide efficient methods and interfaces for managing workout-based notifications. In some embodiments, workout-based notifications can improve user safety. Such techniques can reduce the cognitive burden on a user who access and/or utilize workout-based notifications, thereby enhancing productivity. Further, such techniques can reduce processor and battery power otherwise wasted on redundant user inputs.

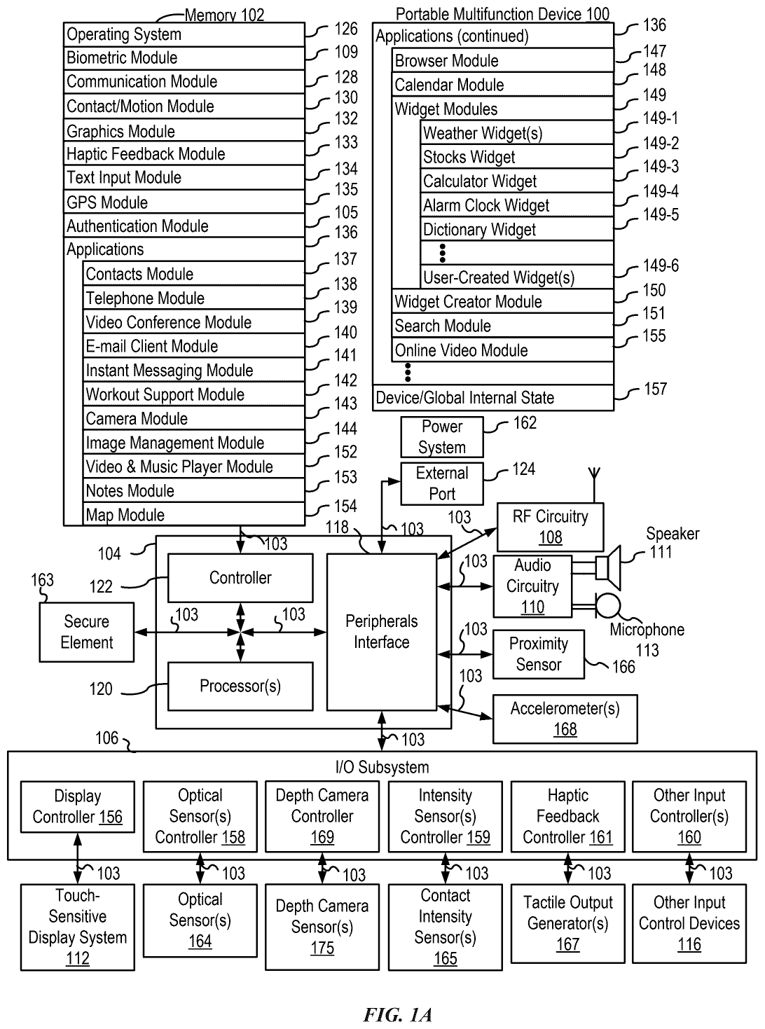

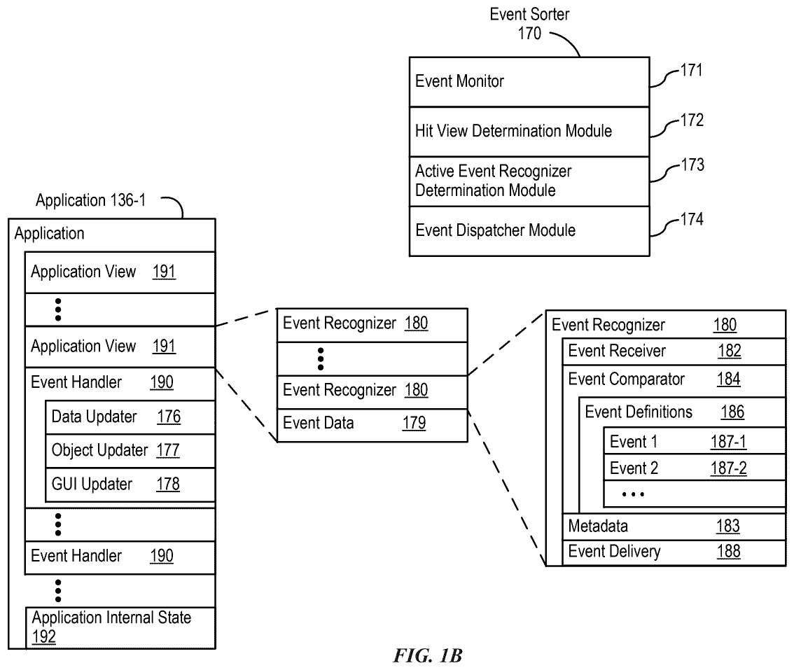

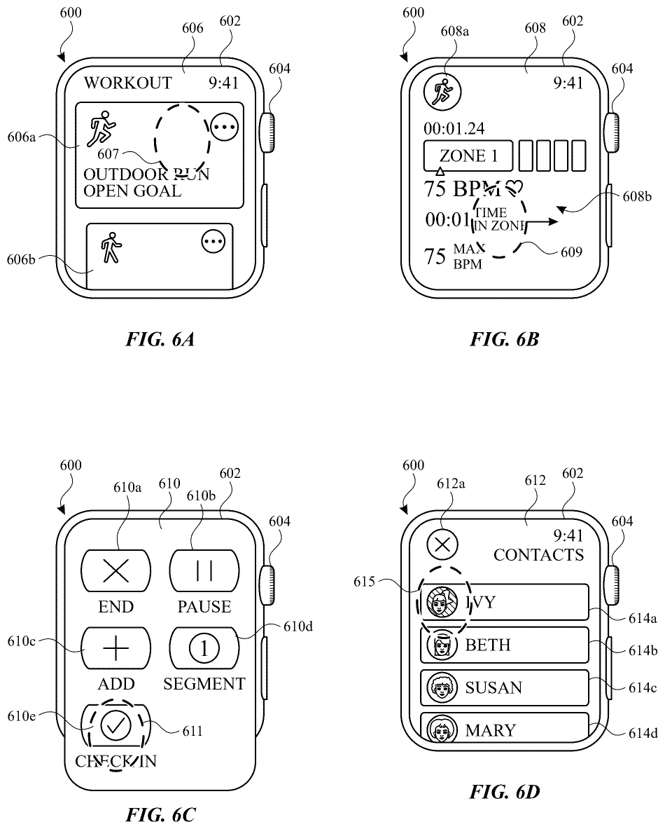

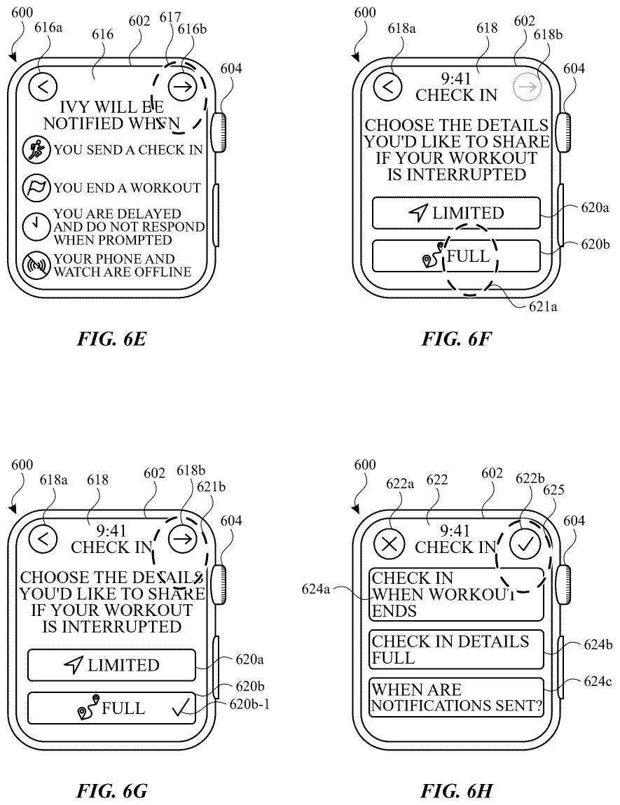

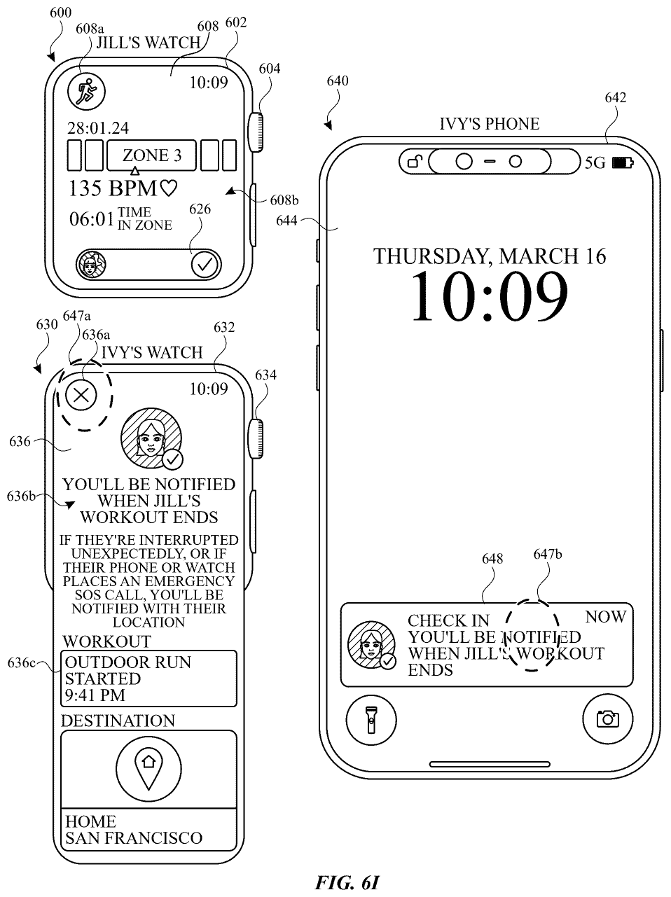

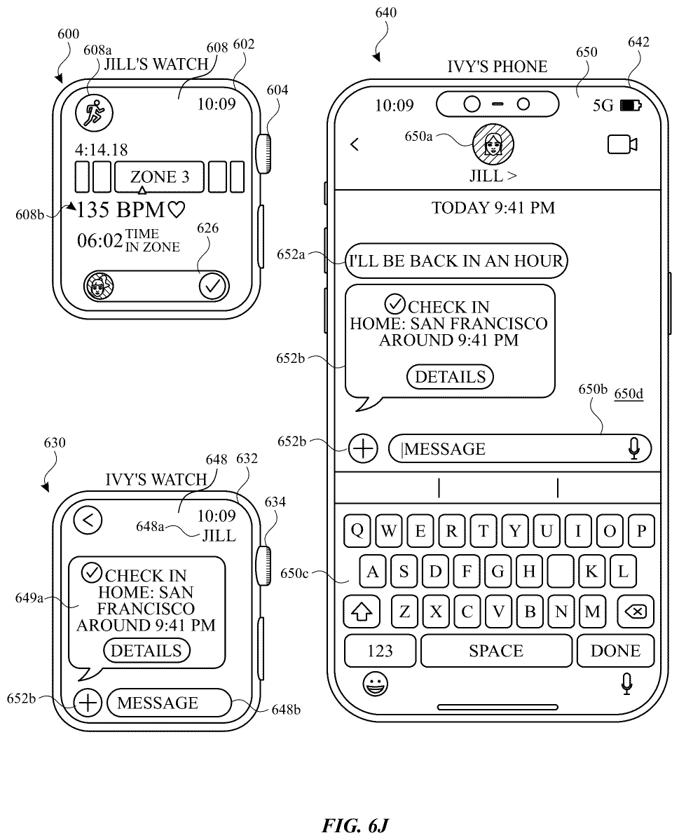

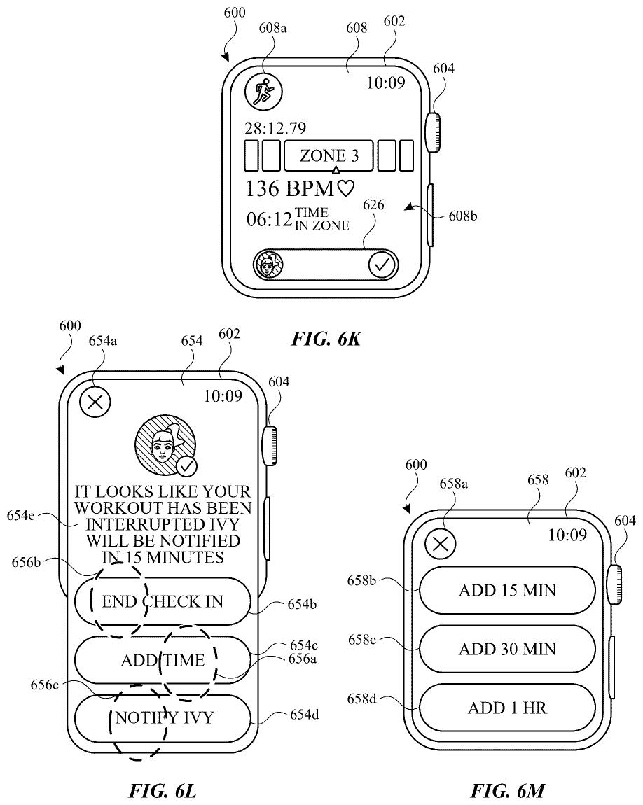

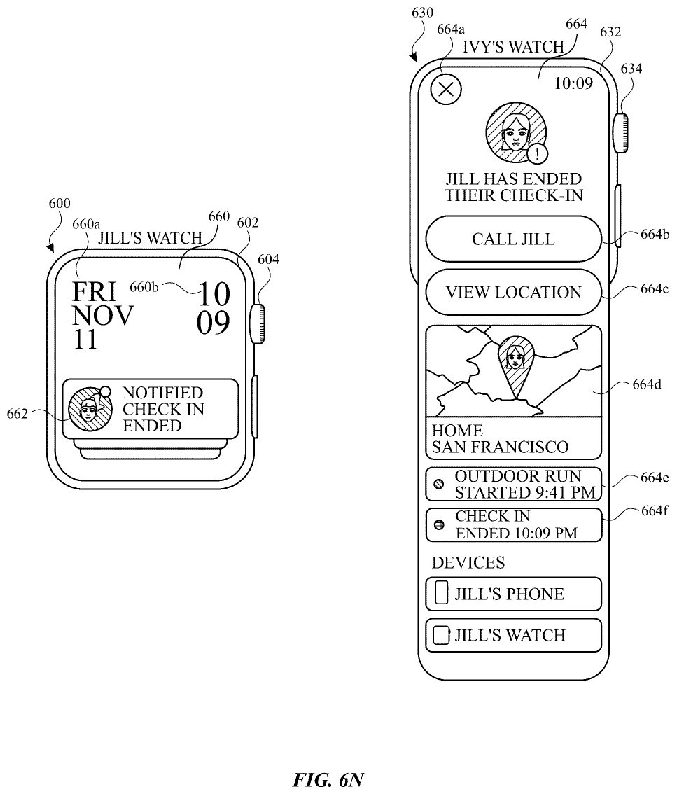

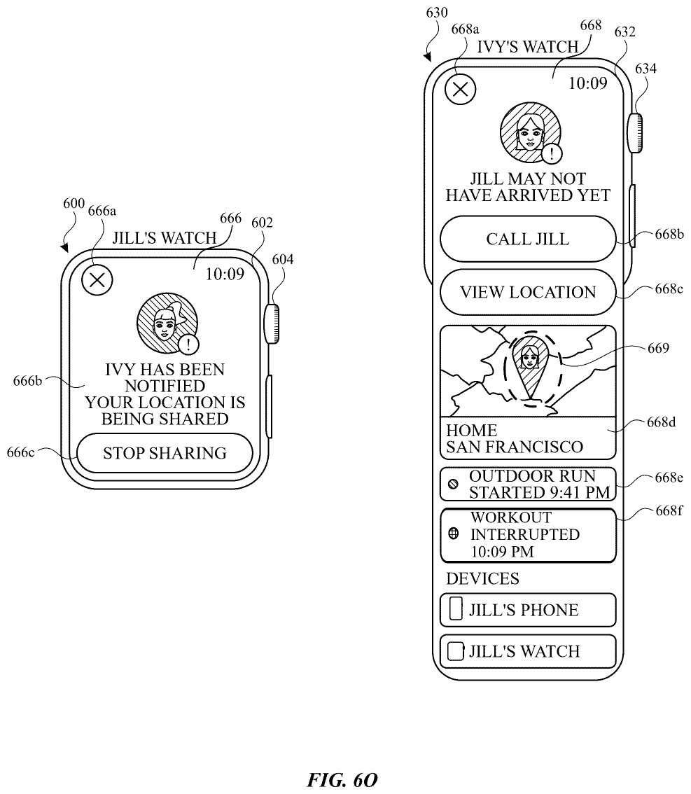

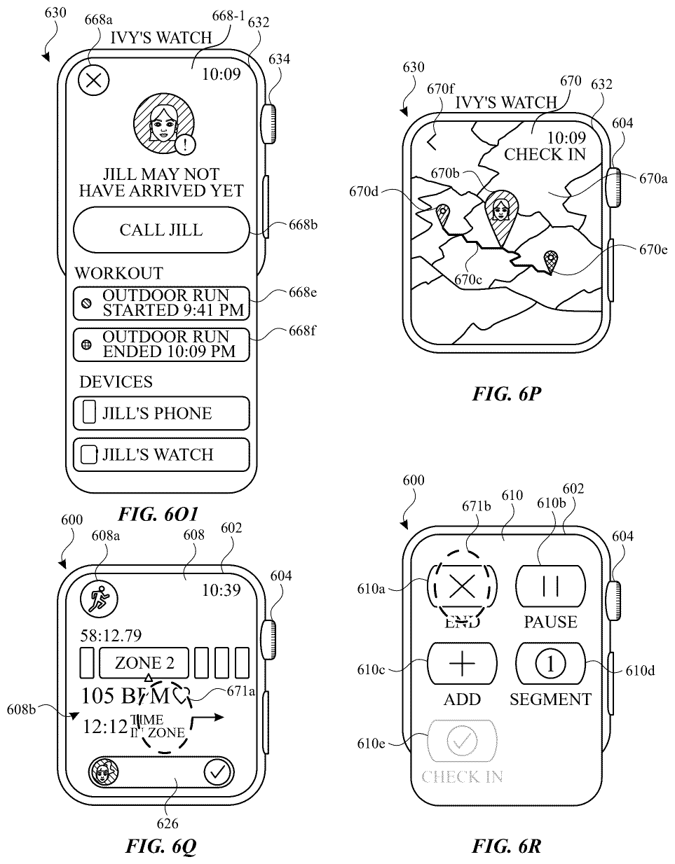

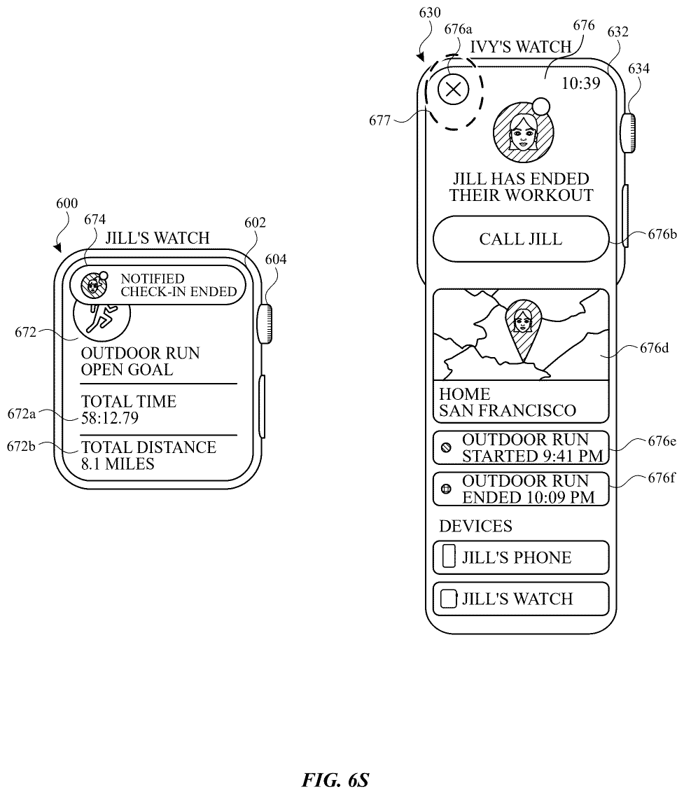

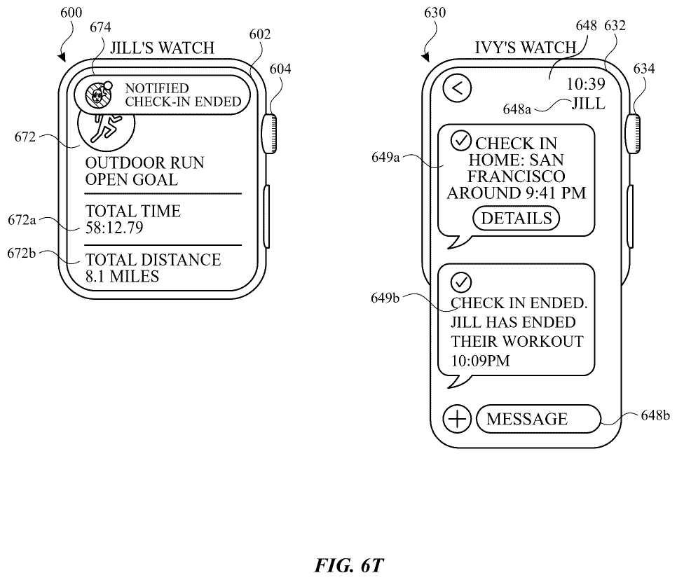

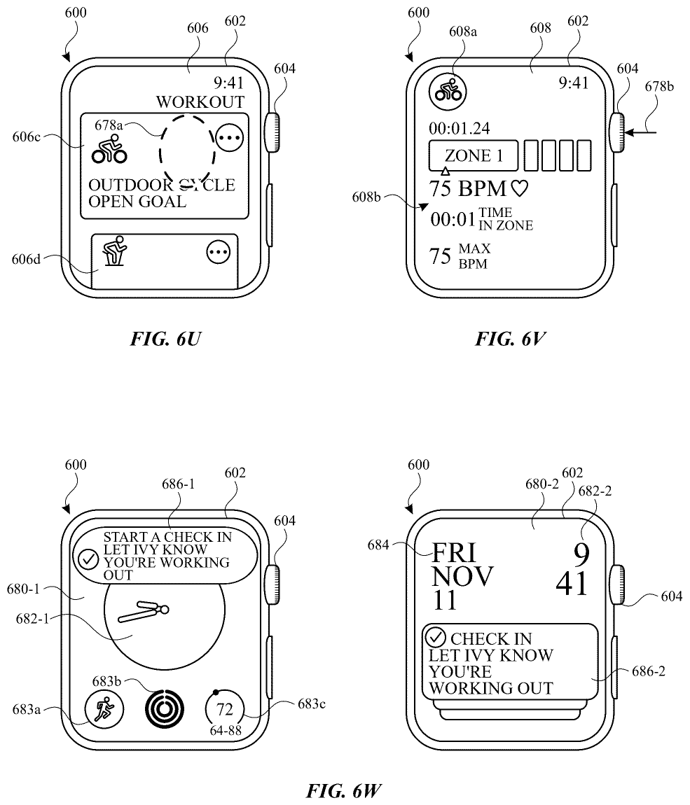

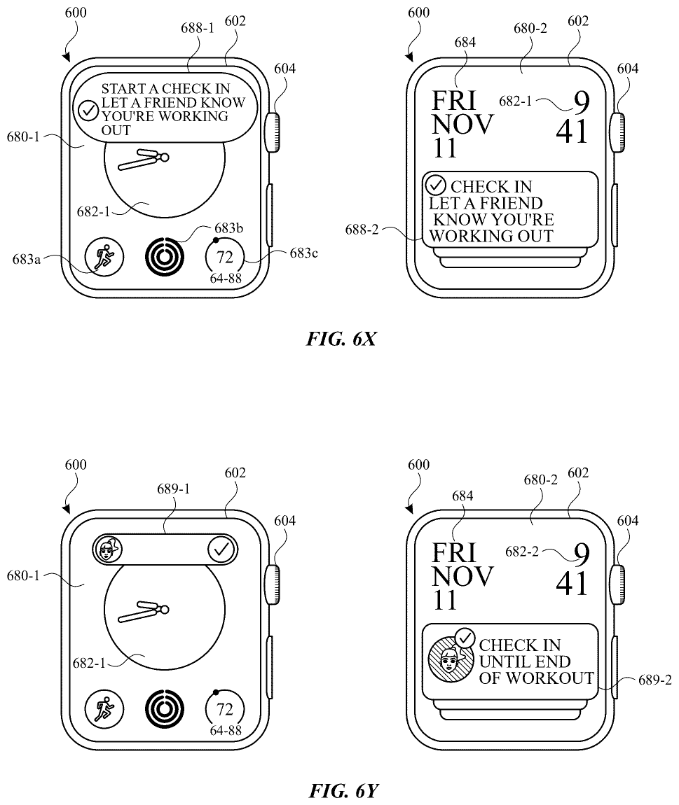

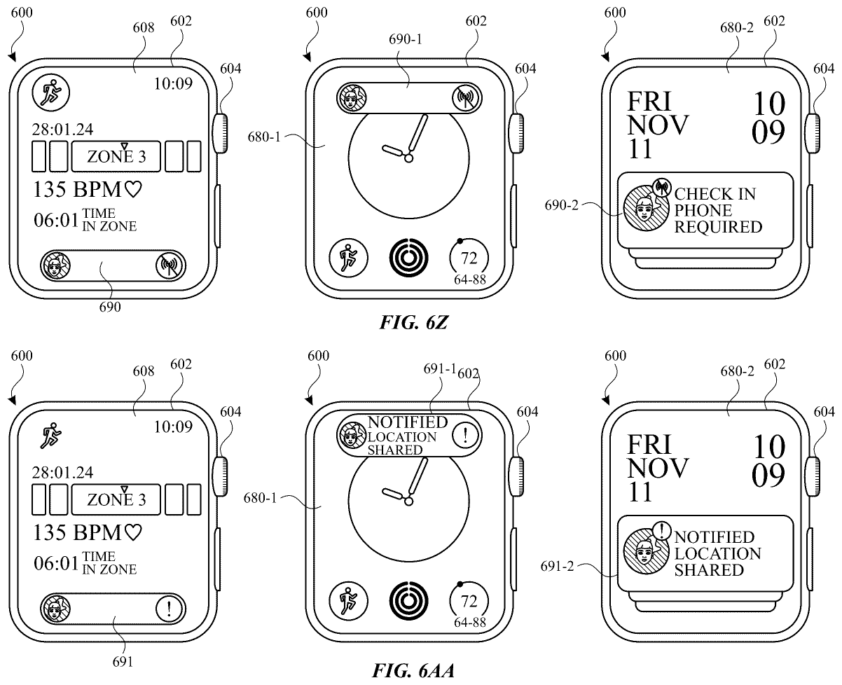

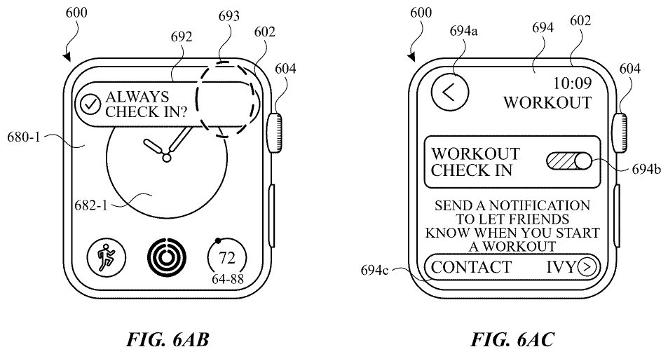

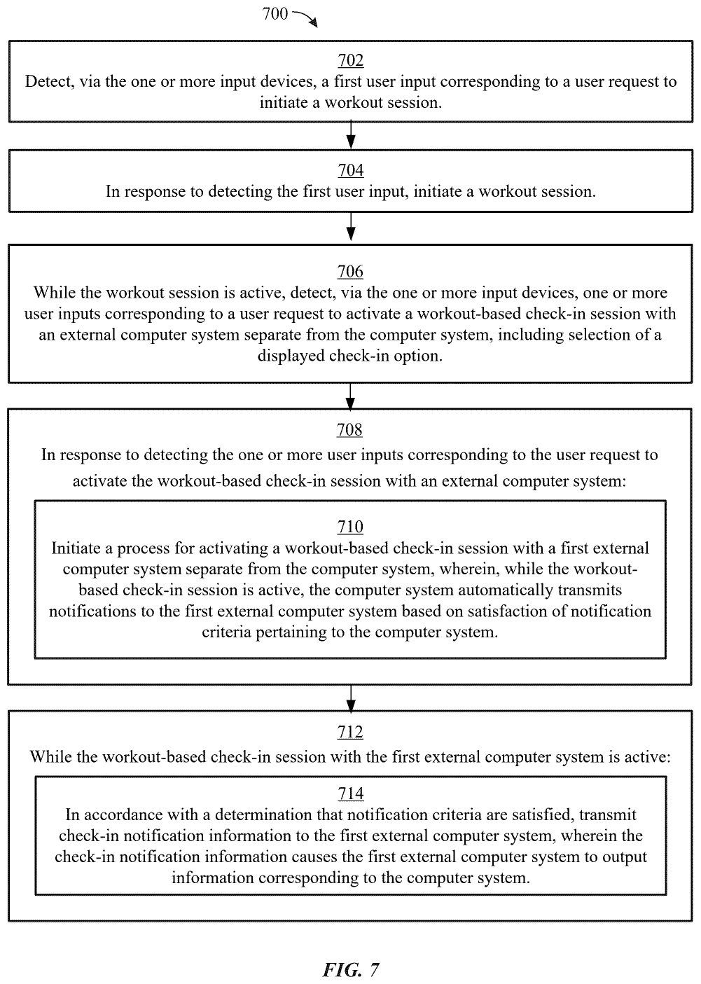

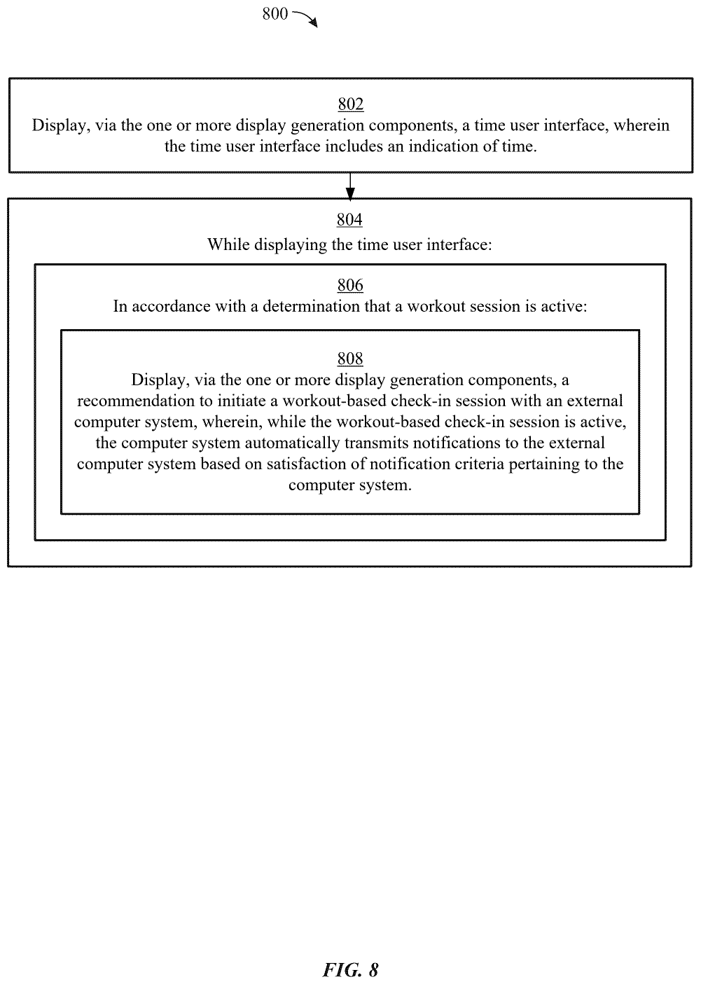

Below, FIGS. 1A-1B, 2, 3A-3G, 4A-4B, and 5A-5B provide a description of exemplary devices for performing the techniques for managing workout-based notifications. FIGS. 6A-6A C illustrate exemplary user interfaces for managing workout-based notifications. FIG. 7 is a flow diagram illustrating methods of managing workout-based notifications in accordance with some embodiments. FIG. 8 is a flow diagram illustrating methods of managing workout-based notifications in accordance with some embodiments. The user interfaces in FIGS. 6A-6A C are used to illustrate the processes described below, including the processes in FIG. 7 and FIG. 8.

The processes described below enhance the operability of the devices and make the user-device interfaces more efficient (e.g., by helping the user to provide proper inputs and reducing user mistakes when operating/interacting with the device) through various techniques, including by providing improved visual feedback to the user, reducing the number of inputs needed to perform an operation, providing additional control options without cluttering the user interface with additional displayed controls, performing an operation when a set of conditions has been met without requiring further user input, and/or additional techniques. These techniques also reduce power usage and improve battery life of the device by enabling the user to use the device more quickly and efficiently.

In addition, in methods described herein where one or more steps are contingent upon one or more conditions having been met, it should be understood that the described method can be repeated in multiple repetitions so that over the course of the repetitions all of the conditions upon which steps in the method are contingent have been met in different repetitions of the method. For example, if a method requires performing a first step if a condition is satisfied, and a second step if the condition is not satisfied, then a person of ordinary skill would appreciate that the claimed steps are repeated until the condition has been both satisfied and not satisfied, in no particular order. Thus, a method described with one or more steps that are contingent upon one or more conditions having been met could be rewritten as a method that is repeated until each of the conditions described in the method has been met. This, however, is not required of system or computer readable medium claims where the system or computer readable medium contains instructions for performing the contingent operations based on the satisfaction of the corresponding one or more conditions and thus is capable of determining whether the contingency has or has not been satisfied without explicitly repeating steps of a method until all of the conditions upon which steps in the method are contingent have been met. A person having ordinary skill in the art would also understand that, similar to a method with contingent steps, a system or computer readable storage medium can repeat the steps of a method as many times as are needed to ensure that all of the contingent steps have been performed.

Although the following description uses terms “first,” “second,” etc. to describe various elements, these elements should not be limited by the terms. In some embodiments, these terms are used to distinguish one element from another. For example, a first touch could be termed a second touch, and, similarly, a second touch could be termed a first touch, without departing from the scope of the various described embodiments. In some embodiments, the first touch and the second touch are two separate references to the same touch. In some embodiments, the first touch and the second touch are both touches, but they are not the same touch.

The terminology used in the description of the various described embodiments herein is for the purpose of describing particular embodiments only and is not intended to be limiting. As used in the description of the various described embodiments and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

The term “if” is, optionally, construed to mean “when” or “upon” or “in response to determining” or “in response to detecting,” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event],” depending on the context.

Embodiments of electronic devices, user interfaces for such devices, and associated processes for using such devices are described. In some embodiments, the device is a portable communications device, such as a mobile telephone, that also contains other functions, such as PDA and/or music player functions. Exemplary embodiments of portable multifunction devices include, without limitation, the iPhone®, iPod Touch®, and iPad® devices from Apple Inc. of Cupertino, California. Other portable electronic devices, such as laptops or tablet computers with touch-sensitive surfaces (e.g., touch screen displays and/or touchpads), are, optionally, used. It should also be understood that, in some embodiments, the device is not a portable communications device, but is a desktop computer with a touch-sensitive surface (e.g., a touch screen display and/or a touchpad). In some embodiments, the electronic device is a computer system that is in communication (e.g., via wireless communication, via wired communication) with a display generation component (e.g., a display device such as a head-mounted display (HM D), a display, a projector, a touch-sensitive display, or other device or component that presents visual content to a user, for example on or in the display generation component itself or produced from the display generation component and visible elsewhere). The display generation component is configured to provide visual output, such as display via a CRT display, display via an LED display, or display via image projection. In some embodiments, the display generation component is integrated with the computer system. In some embodiments, the display generation component is separate from the computer system. As used herein, “displaying” content includes causing to display the content (e.g., video data rendered or decoded by display controller 156) by transmitting, via a wired or wireless connection, data (e.g., image data or video data) to an integrated or external display generation component to visually produce the content.

In the discussion that follows, an electronic device that includes a display and a touch-sensitive surface is described. It should be understood, however, that the electronic device optionally includes one or more other physical user-interface devices, such as a physical keyboard, a mouse, and/or a joystick.

The device typically supports a variety of applications, such as one or more of the following: a drawing application, a presentation application, a word processing application, a website creation application, a disk authoring application, a spreadsheet application, a gaming application, a telephone application, a video conferencing application, an e-mail application, an instant messaging application, a workout support application, a photo management application, a digital camera application, a digital video camera application, a web browsing application, a digital music player application, and/or a digital video player application.

The various applications that are executed on the device optionally use at least one common physical user-interface device, such as the touch-sensitive surface. One or more functions of the touch-sensitive surface as well as corresponding information displayed on the device are, optionally, adjusted and/or varied from one application to the next and/or within a respective application. In this way, a common physical architecture (such as the touch-sensitive surface) of the device optionally supports the variety of applications with user interfaces that are intuitive and transparent to the user.

Attention is now directed toward embodiments of portable devices with touch-sensitive displays. FIG. 1A is a block diagram illustrating portable multifunction device 100 with touch-sensitive display system 112 in accordance with some embodiments. Touch-sensitive display 112 is sometimes called a “touch screen” for convenience and is sometimes known as or called a “touch-sensitive display system.” Device 100 includes memory 102 (which optionally includes one or more computer-readable storage mediums), memory controller 122, one or more processing units (CPUs) 120, peripherals interface 118, RF circuitry 108, audio circuitry 110, speaker 111, microphone 113, input/output (I/O) subsystem 106, other input control devices 116, and external port 124. Device 100 optionally includes one or more optical sensors 164. Device 100 optionally includes one or more contact intensity sensors 165 for detecting intensity of contacts on device 100 (e.g., a touch-sensitive surface such as touch-sensitive display system 112 of device 100). Device 100 optionally includes one or more tactile output generators 167 for generating tactile outputs on device 100 (e.g., generating tactile outputs on a touch-sensitive surface such as touch-sensitive display system 112 of device 100 or touchpad 355 of device 300). These components optionally communicate over one or more communication buses or signal lines 103.

As used in the specification and claims, the term “intensity” of a contact on a touch-sensitive surface refers to the force or pressure (force per unit area) of a contact (e.g., a finger contact) on the touch-sensitive surface, or to a substitute (proxy) for the force or pressure of a contact on the touch-sensitive surface. The intensity of a contact has a range of values that includes at least four distinct values and more typically includes hundreds of distinct values (e.g., at least 256). Intensity of a contact is, optionally, determined (or measured) using various approaches and various sensors or combinations of sensors. For example, one or more force sensors underneath or adjacent to the touch-sensitive surface are, optionally, used to measure force at various points on the touch-sensitive surface. In some implementations, force measurements from multiple force sensors are combined (e.g., a weighted average) to determine an estimated force of a contact. Similarly, a pressure-sensitive tip of a stylus is, optionally, used to determine a pressure of the stylus on the touch-sensitive surface. Alternatively, the size of the contact area detected on the touch-sensitive surface and/or changes thereto, the capacitance of the touch-sensitive surface proximate to the contact and/or changes thereto, and/or the resistance of the touch-sensitive surface proximate to the contact and/or changes thereto are, optionally, used as a substitute for the force or pressure of the contact on the touch-sensitive surface. In some implementations, the substitute measurements for contact force or pressure are used directly to determine whether an intensity threshold has been exceeded (e.g., the intensity threshold is described in units corresponding to the substitute measurements). In some implementations, the substitute measurements for contact force or pressure are converted to an estimated force or pressure, and the estimated force or pressure is used to determine whether an intensity threshold has been exceeded (e.g., the intensity threshold is a pressure threshold measured in units of pressure). Using the intensity of a contact as an attribute of a user input allows for user access to additional device functionality that may otherwise not be accessible by the user on a reduced-size device with limited real estate for displaying affordances (e.g., on a touch-sensitive display) and/or receiving user input (e.g., via a touch-sensitive display, a touch-sensitive surface, or a physical/mechanical control such as a knob or a button).

As used in the specification and claims, the term “tactile output” refers to physical displacement of a device relative to a previous position of the device, physical displacement of a component (e.g., a touch-sensitive surface) of a device relative to another component (e.g., housing) of the device, or displacement of the component relative to a center of mass of the device that will be detected by a user with the user's sense of touch. For example, in situations where the device or the component of the device is in contact with a surface of a user that is sensitive to touch (e.g., a finger, palm, or other part of a user's hand), the tactile output generated by the physical displacement will be interpreted by the user as a tactile sensation corresponding to a perceived change in physical characteristics of the device or the component of the device. For example, movement of a touch-sensitive surface (e.g., a touch-sensitive display or trackpad) is, optionally, interpreted by the user as a “down click” or “up click” of a physical actuator button. In some cases, a user will feel a tactile sensation such as an “down click” or “up click” even when there is no movement of a physical actuator button associated with the touch-sensitive surface that is physically pressed (e.g., displaced) by the user's movements. As another example, movement of the touch-sensitive surface is, optionally, interpreted or sensed by the user as “roughness” of the touch-sensitive surface, even when there is no change in smoothness of the touch-sensitive surface. While such interpretations of touch by a user will be subject to the individualized sensory perceptions of the user, there are many sensory perceptions of touch that are common to a large majority of users. Thus, when a tactile output is described as corresponding to a particular sensory perception of a user (e.g., an “up click,” a “down click,” “roughness”), unless otherwise stated, the generated tactile output corresponds to physical displacement of the device or a component thereof that will generate the described sensory perception for a typical (or average) user.

It should be appreciated that device 100 is only one example of a portable multifunction device, and that device 100 optionally has more or fewer components than shown, optionally combines two or more components, or optionally has a different configuration or arrangement of the components. The various components shown in FIG. 1A are implemented in hardware, software, or a combination of both hardware and software, including one or more signal processing and/or application-specific integrated circuits.

Memory 102 optionally includes high-speed random access memory and optionally also includes non-volatile memory, such as one or more magnetic disk storage devices, flash memory devices, or other non-volatile solid-state memory devices. Memory controller 122 optionally controls access to memory 102 by other components of device 100.

Peripherals interface 118 can be used to couple input and output peripherals of the device to CPU 120 and memory 102. The one or more processors 120 run or execute various software programs (such as computer programs (e.g., including instructions)) and/or sets of instructions stored in memory 102 to perform various functions for device 100 and to process data. In some embodiments, peripherals interface 118, CPU 120, and memory controller 122 are, optionally, implemented on a single chip, such as chip 104. In some other embodiments, they are, optionally, implemented on separate chips.

RF (radio frequency) circuitry 108 receives and sends RF signals, also called electromagnetic signals. RF circuitry 108 converts electrical signals to/from electromagnetic signals and communicates with communications networks and other communications devices via the electromagnetic signals. RF circuitry 108 optionally includes well-known circuitry for performing these functions, including but not limited to an antenna system, an RF transceiver, one or more amplifiers, a tuner, one or more oscillators, a digital signal processor, a CODEC chipset, a subscriber identity module (SIM) card, memory, and so forth. RF circuitry 108 optionally communicates with networks, such as the Internet, also referred to as the World Wide Web (WWW), an intranet and/or a wireless network, such as a cellular telephone network, a wireless local area network (LAN) and/or a metropolitan area network (MAN), and other devices by wireless communication. The RF circuitry 108 optionally includes well-known circuitry for detecting near field communication (NFC) fields, such as by a short-range communication radio. The wireless communication optionally uses any of a plurality of communications standards, protocols, and technologies, including but not limited to Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Bluetooth Low Energy (BTLE), Wireless Fidelity (Wi-Fi) (e.g., IEEE 802.11a, IEEE 802.11b, IEEE 802.11g, IEEE 802.11n, and/or IEEE 802.11ac), voice over Internet Protocol (VoIP), Wi-MAX, a protocol for e-mail (e.g., Internet message access protocol (IMAP) and/or post office protocol (POP)), instant messaging (e.g., extensible messaging and presence protocol (XM PP), Session Initiation Protocol for Instant Messaging and Presence Leveraging Extensions (SIM PLE), Instant Messaging and Presence Service (IMPS)), and/or Short Message Service (SMS), or any other suitable communication protocol, including communication protocols not yet developed as of the filing date of this document.

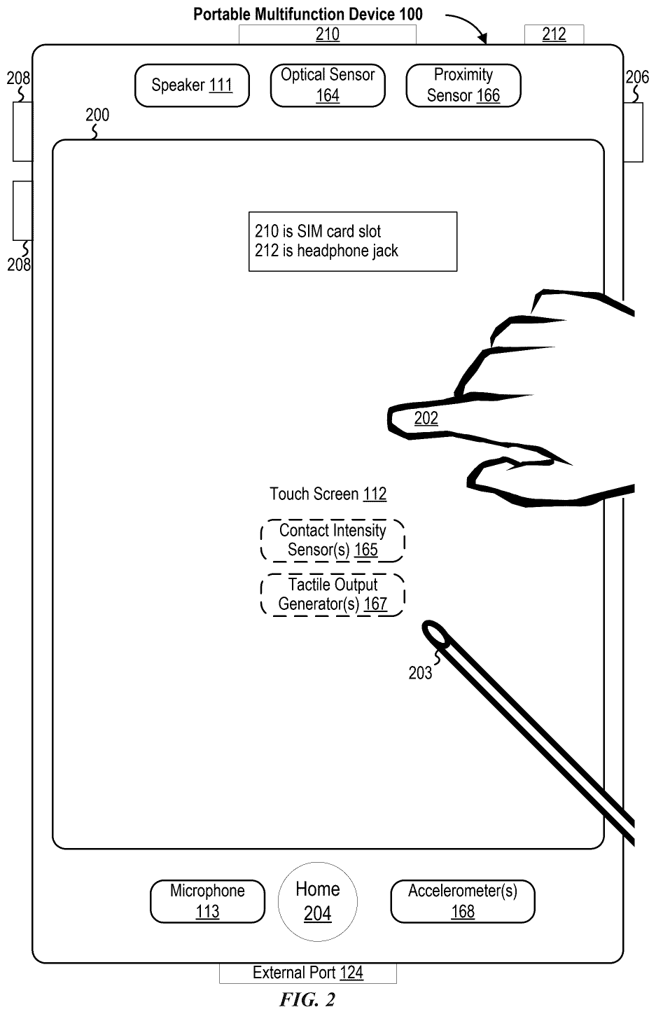

Audio circuitry 110, speaker 111, and microphone 113 provide an audio interface between a user and device 100. Audio circuitry 110 receives audio data from peripherals interface 118, converts the audio data to an electrical signal, and transmits the electrical signal to speaker 111. Speaker 111 converts the electrical signal to human-audible sound waves. Audio circuitry 110 also receives electrical signals converted by microphone 113 from sound waves. Audio circuitry 110 converts the electrical signal to audio data and transmits the audio data to peripherals interface 118 for processing. Audio data is, optionally, retrieved from and/or transmitted to memory 102 and/or RF circuitry 108 by peripherals interface 118. In some embodiments, audio circuitry 110 also includes a headset jack (e.g., 212, FIG. 2). The headset jack provides an interface between audio circuitry 110 and removable audio input/output peripherals, such as output-only headphones or a headset with both output (e.g., a headphone for one or both ears) and input (e.g., a microphone).

I/O subsystem 106 couples input/output peripherals on device 100, such as touch screen 112 and other input control devices 116, to peripherals interface 118. I/O subsystem 106 optionally includes display controller 156, optical sensor controller 158, depth camera controller 169, intensity sensor controller 159, haptic feedback controller 161, and one or more input controllers 160 for other input or control devices. The one or more input controllers 160 receive/send electrical signals from/to other input control devices 116. The other input control devices 116 optionally include physical buttons (e.g., push buttons, rocker buttons, etc.), dials, slider switches, joysticks, click wheels, and so forth. In some embodiments, input controller(s) 160 are, optionally, coupled to any (or none) of the following: a keyboard, an infrared port, a USB port, and a pointer device such as a mouse. The one or more buttons (e.g., 208, FIG. 2) optionally include an up/down button for volume control of speaker 111 and/or microphone 113. The one or more buttons optionally include a push button (e.g., 206, FIG. 2). In some embodiments, the electronic device is a computer system that is in communication (e.g., via wireless communication, via wired communication) with one or more input devices. In some embodiments, the one or more input devices include a touch-sensitive surface (e.g., a trackpad, as part of a touch-sensitive display). In some embodiments, the one or more input devices include one or more camera sensors (e.g., one or more optical sensors 164 and/or one or more depth camera sensors 175), such as for tracking a user's gestures (e.g., hand gestures and/or air gestures) as input. In some embodiments, the one or more input devices are integrated with the computer system. In some embodiments, the one or more input devices are separate from the computer system. In some embodiments, an air gesture is a gesture that is detected without the user touching an input element that is part of the device (or independently of an input element that is a part of the device) and is based on detected motion of a portion of the user's body through the air including motion of the user's body relative to an absolute reference (e.g., an angle of the user's arm relative to the ground or a distance of the user's hand relative to the ground), relative to another portion of the user's body (e.g., movement of a hand of the user relative to a shoulder of the user, movement of one hand of the user relative to another hand of the user, and/or movement of a finger of the user relative to another finger or portion of a hand of the user), and/or absolute motion of a portion of the user's body (e.g., a tap gesture that includes movement of a hand in a predetermined pose by a predetermined amount and/or speed, or a shake gesture that includes a predetermined speed or amount of rotation of a portion of the user's body).

A quick press of the push button optionally disengages a lock of touch screen 112 or optionally begins a process that uses gestures on the touch screen to unlock the device, as described in U.S. patent application Ser. No. 11/322,549, “Unlocking a Device by Performing Gestures on an Unlock Image,” filed Dec. 23, 2005, U.S. Pat. No. 7,657,849, which is hereby incorporated by reference in its entirety. A longer press of the push button (e.g., 206) optionally turns power to device 100 on or off. The functionality of one or more of the buttons are, optionally, user-customizable. Touch screen 112 is used to implement virtual or soft buttons and one or more soft keyboards.

Touch-sensitive display 112 provides an input interface and an output interface between the device and a user. Display controller 156 receives and/or sends electrical signals from/to touch screen 112. Touch screen 112 displays visual output to the user. The visual output optionally includes graphics, text, icons, video, and any combination thereof (collectively termed “graphics”). In some embodiments, some or all of the visual output optionally corresponds to user-interface objects.

Touch screen 112 has a touch-sensitive surface, sensor, or set of sensors that accepts input from the user based on haptic and/or tactile contact. Touch screen 112 and display controller 156 (along with any associated modules and/or sets of instructions in memory 102) detect contact (and any movement or breaking of the contact) on touch screen 112 and convert the detected contact into interaction with user-interface objects (e.g., one or more soft keys, icons, web pages, or images) that are displayed on touch screen 112. In an exemplary embodiment, a point of contact between touch screen 112 and the user corresponds to a finger of the user.

Touch screen 112 optionally uses LCD (liquid crystal display) technology, LPD (light emitting polymer display) technology, or LED (light emitting diode) technology, although other display technologies are used in other embodiments. Touch screen 112 and display controller 156 optionally detect contact and any movement or breaking thereof using any of a plurality of touch sensing technologies now known or later developed, including but not limited to capacitive, resistive, infrared, and surface acoustic wave technologies, as well as other proximity sensor arrays or other elements for determining one or more points of contact with touch screen 112. In an exemplary embodiment, projected mutual capacitance sensing technology is used, such as that found in the iPhone® and iPod Touch® from Apple Inc. of Cupertino, California.

A touch-sensitive display in some embodiments of touch screen 112 is, optionally, analogous to the multi-touch sensitive touchpads described in the following U.S. Pat. No. 6,323,846 (Westerman et al.), U.S. Pat. No. 6,570,557 (Westerman et al.), and/or U.S. Pat. No. 6,677,932 (Westerman), and/or U.S. Patent Publication 2002/0015024A1, each of which is hereby incorporated by reference in its entirety. However, touch screen 112 displays visual output from device 100, whereas touch-sensitive touchpads do not provide visual output.

A touch-sensitive display in some embodiments of touch screen 112 is described in the following applications: (1) U.S. patent application Ser. No. 11/381,313, “Multipoint Touch Surface Controller,” filed May 2, 2006; (2) U.S. patent application Ser. No. 10/840,862, “Multipoint Touchscreen,” filed May 6, 2004; (3) U.S. patent application Ser. No. 10/903,964, “Gestures For Touch Sensitive Input Devices,” filed Jul. 30, 2004; (4) U.S. patent application Ser. No. 11/048,264, “Gestures For Touch Sensitive Input Devices,” filed Jan. 31, 2005; (5) U.S. patent application Ser. No. 11/038,590, “Mode-Based Graphical User Interfaces For Touch Sensitive Input Devices,” filed Jan. 18, 2005; (6) U.S. patent application Ser. No. 11/228,758, “Virtual Input Device Placement On A Touch Screen User Interface,” filed Sep. 16, 2005; (7) U.S. patent application Ser. No. 11/228,700, “Operation Of A Computer With A Touch Screen Interface,” filed Sep. 16, 2005; (8) U.S. patent application Ser. No. 11/228,737, “Activating Virtual Keys Of A Touch-Screen Virtual Keyboard,” filed Sep. 16, 2005; and (9) U.S. patent application Ser. No. 11/367,749, “Multi-Functional Hand-Held Device,” filed Mar. 3, 2006. All of these applications are incorporated by reference herein in their entirety.

Touch screen 112 optionally has a video resolution in excess of 100 dpi. In some embodiments, the touch screen has a video resolution of approximately 160 dpi. The user optionally makes contact with touch screen 112 using any suitable object or appendage, such as a stylus, a finger, and so forth. In some embodiments, the user interface is designed to work primarily with finger-based contacts and gestures, which can be less precise than stylus-based input due to the larger area of contact of a finger on the touch screen. In some embodiments, the device translates the rough finger-based input into a precise pointer/cursor position or command for performing the actions desired by the user.

In some embodiments, in addition to the touch screen, device 100 optionally includes a touchpad for activating or deactivating particular functions. In some embodiments, the touchpad is a touch-sensitive area of the device that, unlike the touch screen, does not display visual output. The touchpad is, optionally, a touch-sensitive surface that is separate from touch screen 112 or an extension of the touch-sensitive surface formed by the touch screen.

Device 100 also includes power system 162 for powering the various components. Power system 162 optionally includes a power management system, one or more power sources (e.g., battery, alternating current (AC)), a recharging system, a power failure detection circuit, a power converter or inverter, a power status indicator (e.g., a light-emitting diode (LED)) and any other components associated with the generation, management and distribution of power in portable devices.

Device 100 optionally also includes secure element 163 for securely storing information. In some embodiments, secure element 163 is a hardware component (e.g., a secure microcontroller chip) configured to securely store data or an algorithm. In some embodiments, secure element 163 provides (e.g., releases) secure information (e.g., payment information (e.g., an account number and/or a transaction-specific dynamic security code), identification information (e.g., credentials of a state-approved digital identification), and/or authentication information (e.g., data generated using a cryptography engine and/or by performing asymmetric cryptography operations)). In some embodiments, secure element 163 provides (or releases) the secure information in response to device 100 receiving authorization, such as a user authentication (e.g., fingerprint authentication; passcode authentication; detecting double-press of a hardware button when device 100 is in an unlocked state, and optionally, while device 100 has been continuously on a user's wrist since device 100 was unlocked by providing authentication credentials to device 100, where the continuous presence of device 100 on the user's wrist is determined by periodically checking that the device is in contact with the user's skin). For example, device 100 detects a fingerprint at a fingerprint sensor (e.g., a fingerprint sensor integrated into a button) of device 100. Device 100 determines whether the detected fingerprint is consistent with an enrolled fingerprint. In accordance with a determination that the fingerprint is consistent with the enrolled fingerprint, secure element 163 provides (e.g., releases) the secure information. In accordance with a determination that the fingerprint is not consistent with the enrolled fingerprint, secure element 163 forgoes providing (e.g., releasing) the secure information.

Device 100 optionally also includes one or more optical sensors 164. FIG. 1A shows an optical sensor coupled to optical sensor controller 158 in I/O subsystem 106. Optical sensor 164 optionally includes charge-coupled device (CCD) or complementary metal-oxide semiconductor (CM OS) phototransistors. Optical sensor 164 receives light from the environment, projected through one or more lenses, and converts the light to data representing an image. In conjunction with imaging module 143 (also called a camera module), optical sensor 164 optionally captures still images or video. In some embodiments, an optical sensor is located on the back of device 100, opposite touch screen display 112 on the front of the device so that the touch screen display is enabled for use as a viewfinder for still and/or video image acquisition. In some embodiments, an optical sensor is located on the front of the device so that the user's image is, optionally, obtained for video conferencing while the user views the other video conference participants on the touch screen display. In some embodiments, the position of optical sensor 164 can be changed by the user (e.g., by rotating the lens and the sensor in the device housing) so that a single optical sensor 164 is used along with the touch screen display for both video conferencing and still and/or video image acquisition.

Device 100 optionally also includes one or more depth camera sensors 175. FIG. 1A shows a depth camera sensor coupled to depth camera controller 169 in I/O subsystem 106. Depth camera sensor 175 receives data from the environment to create a three dimensional model of an object (e.g., a face) within a scene from a viewpoint (e.g., a depth camera sensor). In some embodiments, in conjunction with imaging module 143 (also called a camera module), depth camera sensor 175 is optionally used to determine a depth map of different portions of an image captured by the imaging module 143. In some embodiments, a depth camera sensor is located on the front of device 100 so that the user's image with depth information is, optionally, obtained for video conferencing while the user views the other video conference participants on the touch screen display and to capture selfies with depth map data. In some embodiments, the depth camera sensor 175 is located on the back of device, or on the back and the front of the device 100. In some embodiments, the position of depth camera sensor 175 can be changed by the user (e.g., by rotating the lens and the sensor in the device housing) so that a depth camera sensor 175 is used along with the touch screen display for both video conferencing and still and/or video image acquisition.

Device 100 optionally also includes one or more contact intensity sensors 165. FIG. 1A shows a contact intensity sensor coupled to intensity sensor controller 159 in I/O subsystem 106. Contact intensity sensor 165 optionally includes one or more piezoresistive strain gauges, capacitive force sensors, electric force sensors, piezoelectric force sensors, optical force sensors, capacitive touch-sensitive surfaces, or other intensity sensors (e.g., sensors used to measure the force (or pressure) of a contact on a touch-sensitive surface). Contact intensity sensor 165 receives contact intensity information (e.g., pressure information or a proxy for pressure information) from the environment. In some embodiments, at least one contact intensity sensor is collocated with, or proximate to, a touch-sensitive surface (e.g., touch-sensitive display system 112). In some embodiments, at least one contact intensity sensor is located on the back of device 100, opposite touch screen display 112, which is located on the front of device 100.

Device 100 optionally also includes one or more proximity sensors 166. FIG. 1A shows proximity sensor 166 coupled to peripherals interface 118. Alternately, proximity sensor 166 is, optionally, coupled to input controller 160 in I/O subsystem 106. Proximity sensor 166 optionally performs as described in U.S. patent application Ser. No. 11/241,839, “Proximity Detector In Handheld Device”; Ser. No. 11/240,788, “Proximity Detector In Handheld Device”; Ser. No. 11/620,702, “Using Ambient Light Sensor To Augment Proximity Sensor Output”; Ser. No. 11/586,862, “Automated Response To And Sensing Of User Activity In Portable Devices”; and Ser. No. 11/638,251, “Methods And Systems For Automatic Configuration Of Peripherals,” which are hereby incorporated by reference in their entirety. In some embodiments, the proximity sensor turns off and disables touch screen 112 when the multifunction device is placed near the user's ear (e.g., when the user is making a phone call).

Device 100 optionally also includes one or more tactile output generators 167. FIG. 1A shows a tactile output generator coupled to haptic feedback controller 161 in I/O subsystem 106. Tactile output generator 167 optionally includes one or more electroacoustic devices such as speakers or other audio components and/or electromechanical devices that convert energy into linear motion such as a motor, solenoid, electroactive polymer, piezoelectric actuator, electrostatic actuator, or other tactile output generating component (e.g., a component that converts electrical signals into tactile outputs on the device). Contact intensity sensor 165 receives tactile feedback generation instructions from haptic feedback module 133 and generates tactile outputs on device 100 that are capable of being sensed by a user of device 100. In some embodiments, at least one tactile output generator is collocated with, or proximate to, a touch-sensitive surface (e.g., touch-sensitive display system 112) and, optionally, generates a tactile output by moving the touch-sensitive surface vertically (e.g., in/out of a surface of device 100) or laterally (e.g., back and forth in the same plane as a surface of device 100). In some embodiments, at least one tactile output generator sensor is located on the back of device 100, opposite touch screen display 112, which is located on the front of device 100.

Device 100 optionally also includes one or more accelerometers 168. FIG. 1A shows accelerometer 168 coupled to peripherals interface 118. Alternately, accelerometer 168 is, optionally, coupled to an input controller 160 in I/O subsystem 106. Accelerometer 168 optionally performs as described in U.S. Patent Publication No. 20050190059, “Acceleration-based Theft Detection System for Portable Electronic Devices,” and U.S. Patent Publication No. 20060017692, “Methods And Apparatuses For Operating A Portable Device Based On An Accelerometer,” both of which are incorporated by reference herein in their entirety. In some embodiments, information is displayed on the touch screen display in a portrait view or a landscape view based on an analysis of data received from the one or more accelerometers. Device 100 optionally includes, in addition to accelerometer(s) 168, a magnetometer and a GPS (or GLONASS or other global navigation system) receiver for obtaining information concerning the location and orientation (e.g., portrait or landscape) of device 100.

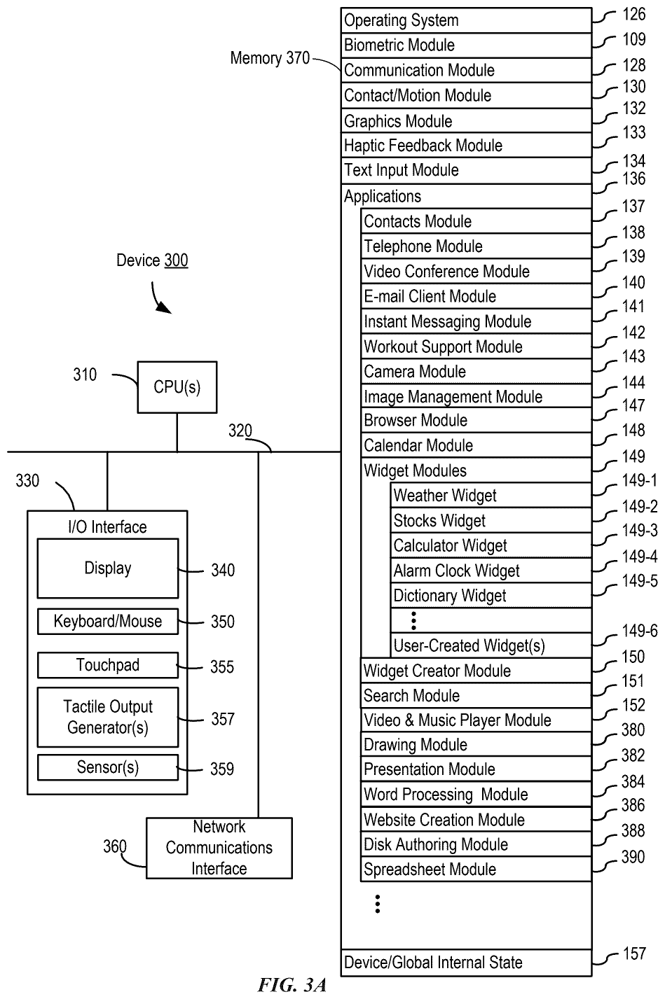

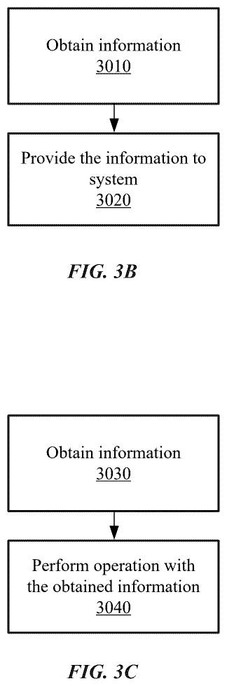

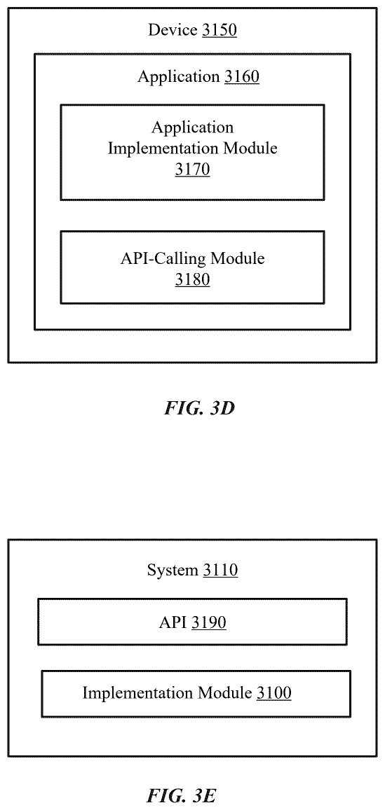

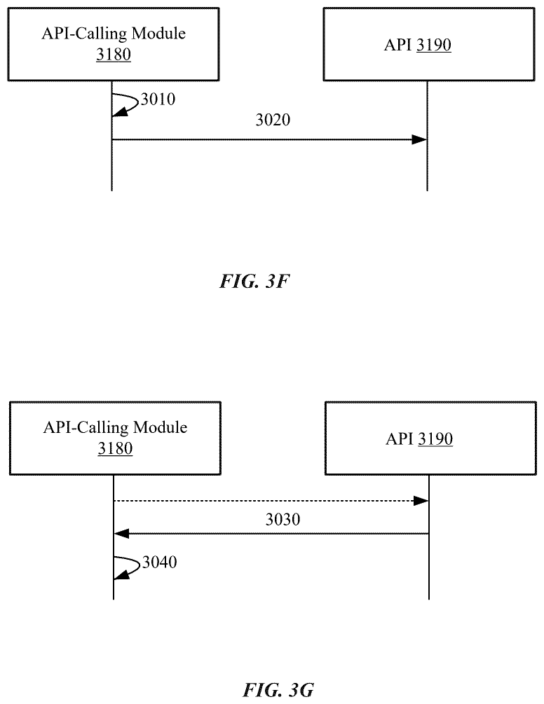

In some embodiments, the software components stored in memory 102 include operating system 126, biometric module 109, communication module (or set of instructions) 128, contact/motion module (or set of instructions) 130, graphics module (or set of instructions) 132, text input module (or set of instructions) 134, Global Positioning System (GPS) module (or set of instructions) 135, authentication module 105, and applications (or sets of instructions) 136. Furthermore, in some embodiments, memory 102 (FIG. 1A) or 370 (FIG. 3A) stores device/global internal state 157, as shown in FIGS. 1A and 3A. Device/global internal state 157 includes one or more of: active application state, indicating which applications, if any, are currently active; display state, indicating what applications, views or other information occupy various regions of touch screen display 112; sensor state, including information obtained from the device's various sensors and input control devices 116; and location information concerning the device's location and/or attitude.

Operating system 126 (e.g., Darwin, RTXC, LINUX, UNIX, OS X, IOS, WINDOWS, or an embedded operating system such as VxWorks) includes various software components and/or drivers for controlling and managing general system tasks (e.g., memory management, storage device control, power management, etc.) and facilitates communication between various hardware and software components.

Communication module 128 facilitates communication with other devices over one or more external ports 124 and also includes various software components for handling data received by RF circuitry 108 and/or external port 124. External port 124 (e.g., Universal Serial Bus (USB), FIREWIRE®, etc.) is adapted for coupling directly to other devices or indirectly over a network (e.g., the Internet, wireless LAN, etc.). In some embodiments, the external port is a multi-pin (e.g., 30-pin) connector that is the same as, or similar to and/or compatible with, the 30-pin connector used on iPod® (trademark of Apple Inc.) devices.

Biometric module 109 optionally stores information about one or more enrolled biometric features (e.g., fingerprint feature information, facial recognition feature information, eye and/or iris feature information) for use to verify whether received biometric information matches the enrolled biometric features. In some embodiments, the information stored about the one or more enrolled biometric features includes data that enables the comparison between the stored information and received biometric information without including enough information to reproduce the enrolled biometric features. In some embodiments, biometric module 109 stores the information about the enrolled biometric features in association with a user account of device 100. In some embodiments, biometric module 109 compares the received biometric information to an enrolled biometric feature to determine whether the received biometric information matches the enrolled biometric feature.

Contact/motion module 130 optionally detects contact with touch screen 112 (in conjunction with display controller 156) and other touch-sensitive devices (e.g., a touchpad or physical click wheel). Contact/motion module 130 includes various software components for performing various operations related to detection of contact, such as determining if contact has occurred (e.g., detecting a finger-down event), determining an intensity of the contact (e.g., the force or pressure of the contact or a substitute for the force or pressure of the contact), determining if there is movement of the contact and tracking the movement across the touch-sensitive surface (e.g., detecting one or more finger-dragging events), and determining if the contact has ceased (e.g., detecting a finger-up event or a break in contact). Contact/motion module 130 receives contact data from the touch-sensitive surface. Determining movement of the point of contact, which is represented by a series of contact data, optionally includes determining speed (magnitude), velocity (magnitude and direction), and/or an acceleration (a change in magnitude and/or direction) of the point of contact. These operations are, optionally, applied to single contacts (e.g., one finger contacts) or to multiple simultaneous contacts (e.g., “multitouch”/multiple finger contacts). In some embodiments, contact/motion module 130 and display controller 156 detect contact on a touchpad.

In some embodiments, contact/motion module 130 uses a set of one or more intensity thresholds to determine whether an operation has been performed by a user (e.g., to determine whether a user has “clicked” on an icon). In some embodiments, at least a subset of the intensity thresholds are determined in accordance with software parameters (e.g., the intensity thresholds are not determined by the activation thresholds of particular physical actuators and can be adjusted without changing the physical hardware of device 100). For example, a mouse “click” threshold of a trackpad or touch screen display can be set to any of a large range of predefined threshold values without changing the trackpad or touch screen display hardware. Additionally, in some implementations, a user of the device is provided with software settings for adjusting one or more of the set of intensity thresholds (e.g., by adjusting individual intensity thresholds and/or by adjusting a plurality of intensity thresholds at once with a system-level click “intensity” parameter).

Contact/motion module 130 optionally detects a gesture input by a user. Different gestures on the touch-sensitive surface have different contact patterns (e.g., different motions, timings, and/or intensities of detected contacts). Thus, a gesture is, optionally, detected by detecting a particular contact pattern. For example, detecting a finger tap gesture includes detecting a finger-down event followed by detecting a finger-up (liftoff) event at the same position (or substantially the same position) as the finger-down event (e.g., at the position of an icon). As another example, detecting a finger swipe gesture on the touch-sensitive surface includes detecting a finger-down event followed by detecting one or more finger-dragging events, and subsequently followed by detecting a finger-up (liftoff) event.

Graphics module 132 includes various known software components for rendering and displaying graphics on touch screen 112 or other display, including components for changing the visual impact (e.g., brightness, transparency, saturation, contrast, or other visual property) of graphics that are displayed. As used herein, the term “graphics” includes any object that can be displayed to a user, including, without limitation, text, web pages, icons (such as user-interface objects including soft keys), digital images, videos, animations, and the like.

In some embodiments, graphics module 132 stores data representing graphics to be used. Each graphic is, optionally, assigned a corresponding code. Graphics module 132 receives, from applications etc., one or more codes specifying graphics to be displayed along with, if necessary, coordinate data and other graphic property data, and then generates screen image data to output to display controller 156.

Haptic feedback module 133 includes various software components for generating instructions used by tactile output generator(s) 167 to produce tactile outputs at one or more locations on device 100 in response to user interactions with device 100.

Text input module 134, which is, optionally, a component of graphics module 132, provides soft keyboards for entering text in various applications (e.g., contacts module 137, e-mail client module 140, IM module 141, browser module 147, and any other application that needs text input).

GPS module 135 determines the location of the device and provides this information for use in various applications (e.g., to telephone module 138 for use in location-based dialing; to camera module 143 as picture/video metadata; and to applications that provide location-based services such as weather widgets, local yellow page widgets, and map/navigation widgets).

Authentication module 105 determines whether a requested operation (e.g., requested by an application of applications 136) is authorized to be performed. In some embodiments, authentication module 105 receives for an operation to be perform that optionally requires authentication. Authentication module 105 determines whether the operation is authorized to be performed, such as based on a series of factors, including the lock status of device 100, the location of device 100, whether a security delay has elapsed, whether received biometric information matches enrolled biometric features, and/or other factors. Once authentication module 105 determines that the operation is authorized to be performed, authentication module 105 triggers performance of the operation.

Applications 136 optionally include the following modules (or sets of instructions), or a subset or superset thereof:
Contacts module 137 (sometimes called an address book or contact list);
Telephone module 138;
Video conference module 139;
E-mail client module 140;
Instant messaging (IM) module 141;
Workout support module 142;
Camera module 143 for still and/or video images;
Image management module 144;
Video player module;
Music player module;
Browser module 147;
Calendar module 148;
Widget modules 149, which optionally include one or more of: weather widget 149-1, stocks widget 149-2, calculator widget 149-3, alarm clock widget 149-4, dictionary widget 149-5, and other widgets obtained by the user, as well as user-created widgets 149-6;
Widget creator module 150 for making user-created widgets 149-6;
Search module 151;
Video and music player module 152, which merges video player module and music player module;
Notes module 153;
Map module 154; and/or
Online video module 155.

Examples of other applications 136 that are, optionally, stored in memory 102 include other word processing applications, other image editing applications, drawing applications, presentation applications, JAVA-enabled applications, encryption, digital rights management, voice recognition, and voice replication.

In conjunction with touch screen 112, display controller 156, contact/motion module 130, graphics module 132, and text input module 134, contacts module 137 are, optionally, used to manage an address book or contact list (e.g., stored in application internal state 192 of contacts module 137 in memory 102 or memory 370), including: adding name(s) to the address book; deleting name(s) from the address book; associating telephone number(s), e-mail address(es), physical address(es) or other information with a name; associating an image with a name; categorizing and sorting names; providing telephone numbers or e-mail addresses to initiate and/or facilitate communications by telephone module 138, video conference module 139, e-mail client module 140, or IM module 141; and so forth.

In conjunction with RF circuitry 108, audio circuitry 110, speaker 111, microphone 113, touch screen 112, display controller 156, contact/motion module 130, graphics module 132, and text input module 134, telephone module 138 are optionally, used to enter a sequence of characters corresponding to a telephone number, access one or more telephone numbers in contacts module 137, modify a telephone number that has been entered, dial a respective telephone number, conduct a conversation, and disconnect or hang up when the conversation is completed. As noted above, the wireless communication optionally uses any of a plurality of communications standards, protocols, and technologies.

In conjunction with RF circuitry 108, audio circuitry 110, speaker 111, microphone 113, touch screen 112, display controller 156, optical sensor 164, optical sensor controller 158, contact/motion module 130, graphics module 132, text input module 134, contacts module 137, and telephone module 138, video conference module 139 includes executable instructions to initiate, conduct, and terminate a video conference between a user and one or more other participants in accordance with user instructions.

In conjunction with RF circuitry 108, touch screen 112, display controller 156, contact/motion module 130, graphics module 132, and text input module 134, e-mail client module 140 includes executable instructions to create, send, receive, and manage e-mail in response to user instructions. In conjunction with image management module 144, e-mail client module 140 makes it very easy to create and send e-mails with still or video images taken with camera module 143.

In conjunction with RF circuitry 108, touch screen 112, display controller 156, contact/motion module 130, graphics module 132, and text input module 134, the instant messaging module 141 includes executable instructions to enter a sequence of characters corresponding to an instant message, to modify previously entered characters, to transmit a respective instant message (for example, using a Short Message Service (SMS) or Multimedia Message Service (MMS) protocol for telephony-based instant messages or using XM PP, SIM PLE, or IMPS for Internet-based instant messages), to receive instant messages, and to view received instant messages. In some embodiments, transmitted and/or received instant messages optionally include graphics, photos, audio files, video files and/or other attachments as are supported in an MMS and/or an Enhanced Messaging Service (EMS). As used herein, “instant messaging” refers to both telephony-based messages (e.g., messages sent using SMS or MMS) and Internet-based messages (e.g., messages sent using XMPP, SIM PLE, or IMPS).

In conjunction with RF circuitry 108, touch screen 112, display controller 156, contact/motion module 130, graphics module 132, text input module 134, GPS module 135, map module 154, and music player module, workout support module 142 includes executable instructions to create workouts (e.g., with time, distance, and/or calorie burning goals); communicate with workout sensors (sports devices); receive workout sensor data; calibrate sensors used to monitor a workout; select and play music for a workout; and display, store, and transmit workout data.

In conjunction with touch screen 112, display controller 156, optical sensor(s) 164, optical sensor controller 158, contact/motion module 130, graphics module 132, and image management module 144, camera module 143 includes executable instructions to capture still images or video (including a video stream) and store them into memory 102, modify characteristics of a still image or video, or delete a still image or video from memory 102.

In conjunction with touch screen 112, display controller 156, contact/motion module 130, graphics module 132, text input module 134, and camera module 143, image management module 144 includes executable instructions to arrange, modify (e.g., edit), or otherwise manipulate, label, delete, present (e.g., in a digital slide show or album), and store still and/or video images.

In conjunction with RF circuitry 108, touch screen 112, display controller 156, contact/motion module 130, graphics module 132, and text input module 134, browser module 147 includes executable instructions to browse the Internet in accordance with user instructions, including searching, linking to, receiving, and displaying web pages or portions thereof, as well as attachments and other files linked to web pages.

In conjunction with RF circuitry 108, touch screen 112, display controller 156, contact/motion module 130, graphics module 132, text input module 134, e-mail client module 140, and browser module 147, calendar module 148 includes executable instructions to create, display, modify, and store calendars and data associated with calendars (e.g., calendar entries, to-do lists, etc.) in accordance with user instructions.

In conjunction with RF circuitry 108, touch screen 112, display controller 156, contact/motion module 130, graphics module 132, text input module 134, and browser module 147, widget modules 149 are mini-applications that are, optionally, downloaded and used by a user (e.g., weather widget 149-1, stocks widget 149-2, calculator widget 149-3, alarm clock widget 149-4, and dictionary widget 149-5) or created by the user (e.g., user-created widget 149-6). In some embodiments, a widget includes an HTML (Hypertext Markup Language) file, a CSS (Cascading Style Sheets) file, and a JavaScript® file. In some embodiments, a widget includes an XML (Extensible Markup Language) file and a JavaScript® file (e.g., Yahoo!® Widgets).

In conjunction with RF circuitry 108, touch screen 112, display controller 156, contact/motion module 130, graphics module 132, text input module 134, and browser module 147, the widget creator module 150 are, optionally, used by a user to create widgets (e.g., turning a user-specified portion of a web page into a widget).

In conjunction with touch screen 112, display controller 156, contact/motion module 130, graphics module 132, and text input module 134, search module 151 includes executable instructions to search for text, music, sound, image, video, and/or other files in memory 102 that match one or more search criteria (e.g., one or more user-specified search terms) in accordance with user instructions.

In conjunction with touch screen 112, display controller 156, contact/motion module 130, graphics module 132, audio circuitry 110, speaker 111, RF circuitry 108, and browser module 147, video and music player module 152 includes executable instructions that allow the user to download and play back recorded music and other sound files stored in one or more file formats, such as MP3 or AAC files, and executable instructions to display, present, or otherwise play back videos (e.g., on touch screen 112 or on an external, connected display via external port 124). In some embodiments, device 100 optionally includes the functionality of an MP3 player, such as an iPod (trademark of Apple Inc.).

In conjunction with touch screen 112, display controller 156, contact/motion module 130, graphics module 132, and text input module 134, notes module 153 includes executable instructions to create and manage notes, to-do lists, and the like in accordance with user instructions.

In conjunction with RF circuitry 108, touch screen 112, display controller 156, contact/motion module 130, graphics module 132, text input module 134, GPS module 135, and browser module 147, map module 154 are, optionally, used to receive, display, modify, and store maps and data associated with maps (e.g., driving directions, data on stores and other points of interest at or near a particular location, and other location-based data) in accordance with user instructions.

In conjunction with touch screen 112, display controller 156, contact/motion module 130, graphics module 132, audio circuitry 110, speaker 111, RF circuitry 108, text input module 134, e-mail client module 140, and browser module 147, online video module 155 includes instructions that allow the user to access, browse, receive (e.g., by streaming and/or download), play back (e.g., on the touch screen or on an external, connected display via external port 124), send an e-mail with a link to a particular online video, and otherwise manage online videos in one or more file formats, such as H.264. In some embodiments, instant messaging module 141, rather than e-mail client module 140, is used to send a link to a particular online video. Additional description of the online video application can be found in U.S. Provisional Patent Application No. 60/936,562, “Portable Multifunction Device, Method, and Graphical User Interface for Playing Online Videos,” filed Jun. 20, 2007, and U.S. patent application Ser. No. 11/968,067, “Portable Multifunction Device, Method, and Graphical User Interface for Playing Online Videos,” filed Dec. 31, 2007, the contents of which are hereby incorporated by reference in their entirety.

Each of the above-identified modules and applications corresponds to a set of executable instructions for performing one or more functions described above and the methods described in this application (e.g., the computer-implemented methods and other information processing methods described herein). These modules (e.g., sets of instructions) need not be implemented as separate software programs (such as computer programs (e.g., including instructions)), procedures, or modules, and thus various subsets of these modules are, optionally, combined or otherwise rearranged in various embodiments. For example, video player module is, optionally, combined with music player module into a single module (e.g., video and music player module 152, FIG. 1A). In some embodiments, memory 102 optionally stores a subset of the modules and data structures identified above. Furthermore, memory 102 optionally stores additional modules and data structures not described above.

In some embodiments, device 100 is a device where operation of a predefined set of functions on the device is performed exclusively through a touch screen and/or a touchpad. By using a touch screen and/or a touchpad as the primary input control device for operation of device 100, the number of physical input control devices (such as push buttons, dials, and the like) on device 100 is, optionally, reduced.

The predefined set of functions that are performed exclusively through a touch screen and/or a touchpad optionally include navigation between user interfaces. In some embodiments, the touchpad, when touched by the user, navigates device 100 to a main, home, or root menu from any user interface that is displayed on device 100. In such embodiments, a “menu button” is implemented using a touchpad. In some other embodiments, the menu button is a physical push button or other physical input control device instead of a touchpad.

FIG. 1B is a block diagram illustrating exemplary components for event handling in accordance with some embodiments. In some embodiments, memory 102 (FIG. 1A) or 370 (FIG. 3A) includes event sorter 170 (e.g., in operating system 126) and a respective application 136-1 (e.g., any of the aforementioned applications 137-151, 155, 380-390).

Event sorter 170 receives event information and determines the application 136-1 and application view 191 of application 136-1 to which to deliver the event information. Event sorter 170 includes event monitor 171 and event dispatcher module 174. In some embodiments, application 136-1 includes application internal state 192, which indicates the current application view(s) displayed on touch-sensitive display 112 when the application is active or executing. In some embodiments, device/global internal state 157 is used by event sorter 170 to determine which application(s) is (are) currently active, and application internal state 192 is used by event sorter 170 to determine application views 191 to which to deliver event information.

In some embodiments, application internal state 192 includes additional information, such as one or more of: resume information to be used when application 136-1 resumes execution, user interface state information that indicates information being displayed or that is ready for display by application 136-1, a state queue for enabling the user to go back to a prior state or view of application 136-1, and a redo/undo queue of previous actions taken by the user.

Event monitor 171 receives event information from peripherals interface 118. Event information includes information about a sub-event (e.g., a user touch on touch-sensitive display 112, as part of a multi-touch gesture). Peripherals interface 118 transmits information it receives from I/O subsystem 106 or a sensor, such as proximity sensor 166, accelerometer(s) 168, and/or microphone 113 (through audio circuitry 110). Information that peripherals interface 118 receives from I/O subsystem 106 includes information from touch-sensitive display 112 or a touch-sensitive surface.

In some embodiments, event monitor 171 sends requests to the peripherals interface 118 at predetermined intervals. In response, peripherals interface 118 transmits event information. In other embodiments, peripherals interface 118 transmits event information only when there is a significant event (e.g., receiving an input above a predetermined noise threshold and/or for more than a predetermined duration).

In some embodiments, event sorter 170 also includes a hit view determination module 172 and/or an active event recognizer determination module 173.

Hit view determination module 172 provides software procedures for determining where a sub-event has taken place within one or more views when touch-sensitive display 112 displays more than one view. Views are made up of controls and other elements that a user can see on the display.

Another aspect of the user interface associated with an application is a set of views, sometimes herein called application views or user interface windows, in which information is displayed and touch-based gestures occur. The application views (of a respective application) in which a touch is detected optionally correspond to programmatic levels within a programmatic or view hierarchy of the application. For example, the lowest level view in which a touch is detected is, optionally, called the hit view, and the set of events that are recognized as proper inputs are, optionally, determined based, at least in part, on the hit view of the initial touch that begins a touch-based gesture.

Hit view determination module 172 receives information related to sub-events of a touch-based gesture. When an application has multiple views organized in a hierarchy, hit view determination module 172 identifies a hit view as the lowest view in the hierarchy which should handle the sub-event. In most circumstances, the hit view is the lowest level view in which an initiating sub-event occurs (e.g., the first sub-event in the sequence of sub-events that form an event or potential event). Once the hit view is identified by the hit view determination module 172, the hit view typically receives all sub-events related to the same touch or input source for which it was identified as the hit view.

Active event recognizer determination module 173 determines which view or views within a view hierarchy should receive a particular sequence of sub-events. In some embodiments, active event recognizer determination module 173 determines that only the hit view should receive a particular sequence of sub-events. In other embodiments, active event recognizer determination module 173 determines that all views that include the physical location of a sub-event are actively involved views, and therefore determines that all actively involved views should receive a particular sequence of sub-events. In other embodiments, even if touch sub-events were entirely confined to the area associated with one particular view, views higher in the hierarchy would still remain as actively involved views.

Event dispatcher module 174 dispatches the event information to an event recognizer (e.g., event recognizer 180). In embodiments including active event recognizer determination module 173, event dispatcher module 174 delivers the event information to an event recognizer determined by active event recognizer determination module 173. In some embodiments, event dispatcher module 174 stores in an event queue the event information, which is retrieved by a respective event receiver 182.

In some embodiments, operating system 126 includes event sorter 170. Alternatively, application 136-1 includes event sorter 170. In yet other embodiments, event sorter 170 is a stand-alone module, or a part of another module stored in memory 102, such as contact/motion module 130.

In some embodiments, application 136-1 includes a plurality of event handlers 190 and one or more application views 191, each of which includes instructions for handling touch events that occur within a respective view of the application's user interface. Each application view 191 of the application 136-1 includes one or more event recognizers 180. Typically, a respective application view 191 includes a plurality of event recognizers 180. In other embodiments, one or more of event recognizers 180 are part of a separate module, such as a user interface kit or a higher level object from which application 136-1 inherits methods and other properties. In some embodiments, a respective event handler 190 includes one or more of: data updater 176, object updater 177, GUI updater 178, and/or event data 179 received from event sorter 170. Event handler 190 optionally utilizes or calls data updater 176, object updater 177, or GUI updater 178 to update the application internal state 192. Alternatively, one or more of the application views 191 include one or more respective event handlers 190. Also, in some embodiments, one or more of data updater 176, object updater 177, and GUI updater 178 are included in a respective application view 191.

A respective event recognizer 180 receives event information (e.g., event data 179) from event sorter 170 and identifies an event from the event information. Event recognizer 180 includes event receiver 182 and event comparator 184. In some embodiments, event recognizer 180 also includes at least a subset of: metadata 183, and event delivery instructions 188 (which optionally include sub-event delivery instructions).

Event receiver 182 receives event information from event sorter 170. The event information includes information about a sub-event, for example, a touch or a touch movement. Depending on the sub-event, the event information also includes additional information, such as location of the sub-event. When the sub-event concerns motion of a touch, the event information optionally also includes speed and direction of the sub-event. In some embodiments, events include rotation of the device from one orientation to another (e.g., from a portrait orientation to a landscape orientation, or vice versa), and the event information includes corresponding information about the current orientation (also called device attitude) of the device.

Event comparator 184 compares the event information to predefined event or sub-event definitions and, based on the comparison, determines an event or sub-event, or determines or updates the state of an event or sub-event. In some embodiments, event comparator 184 includes event definitions 186. Event definitions 186 contain definitions of events (e.g., predefined sequences of sub-events), for example, event 1 (187-1), event 2 (187-2), and others. In some embodiments, sub-events in an event (e.g., 187-1 and/or 187-2) include, for example, touch begin, touch end, touch movement, touch cancellation, and multiple touching. In one example, the definition for event 1 (187-1) is a double tap on a displayed object. The double tap, for example, comprises a first touch (touch begin) on the displayed object for a predetermined phase, a first liftoff (touch end) for a predetermined phase, a second touch (touch begin) on the displayed object for a predetermined phase, and a second liftoff (touch end) for a predetermined phase. In another example, the definition for event 2 (187-2) is a dragging on a displayed object. The dragging, for example, comprises a touch (or contact) on the displayed object for a predetermined phase, a movement of the touch across touch-sensitive display 112, and liftoff of the touch (touch end). In some embodiments, the event also includes information for one or more associated event handlers 190.

In some embodiments, event definitions 186 include a definition of an event for a respective user-interface object. In some embodiments, event comparator 184 performs a hit test to determine which user-interface object is associated with a sub-event. For example, in an application view in which three user-interface objects are displayed on touch-sensitive display 112, when a touch is detected on touch-sensitive display 112, event comparator 184 performs a hit test to determine which of the three user-interface objects is associated with the touch (sub-event). If each displayed object is associated with a respective event handler 190, the event comparator uses the result of the hit test to determine which event handler 190 should be activated. For example, event comparator 184 selects an event handler associated with the sub-event and the object triggering the hit test.

In some embodiments, the definition for a respective event (187) also includes delayed actions that delay delivery of the event information until after it has been determined whether the sequence of sub-events does or does not correspond to the event recognizer's event type.

When a respective event recognizer 180 determines that the series of sub-events do not match any of the events in event definitions 186, the respective event recognizer 180 enters an event impossible, event failed, or event ended state, after which it disregards subsequent sub-events of the touch-based gesture. In this situation, other event recognizers, if any, that remain active for the hit view continue to track and process sub-events of an ongoing touch-based gesture.

In some embodiments, a respective event recognizer 180 includes metadata 183 with configurable properties, flags, and/or lists that indicate how the event delivery system should perform sub-event delivery to actively involved event recognizers. In some embodiments, metadata 183 includes configurable properties, flags, and/or lists that indicate how event recognizers interact, or are enabled to interact, with one another. In some embodiments, metadata 183 includes configurable properties, flags, and/or lists that indicate whether sub-events are delivered to varying levels in the view or programmatic hierarchy.

In some embodiments, a respective event recognizer 180 activates event handler 190 associated with an event when one or more particular sub-events of an event are recognized. In some embodiments, a respective event recognizer 180 delivers event information associated with the event to event handler 190. Activating an event handler 190 is distinct from sending (and deferred sending) sub-events to a respective hit view. In some embodiments, event recognizer 180 throws a flag associated with the recognized event, and event handler 190 associated with the flag catches the flag and performs a predefined process.

In some embodiments, event delivery instructions 188 include sub-event delivery instructions that deliver event information about a sub-event without activating an event handler. Instead, the sub-event delivery instructions deliver event information to event handlers associated with the series of sub-events or to actively involved views. Event handlers associated with the series of sub-events or with actively involved views receive the event information and perform a predetermined process.

In some embodiments, data updater 176 creates and updates data used in application 136-1. For example, data updater 176 updates the telephone number used in contacts module 137, or stores a video file used in video player module. In some embodiments, object updater 177 creates and updates objects used in application 136-1. For example, object updater 177 creates a new user-interface object or updates the position of a user-interface object. GUI updater 178 updates the GUI. For example, GUI updater 178 prepares display information and sends it to graphics module 132 for display on a touch-sensitive display.

In some embodiments, event handler(s) 190 includes or has access to data updater 176, object updater 177, and GUI updater 178. In some embodiments, data updater 176, object updater 177, and GUI updater 178 are included in a single module of a respective application 136-1 or application view 191. In other embodiments, they are included in two or more software modules.

It shall be understood that the foregoing discussion regarding event handling of user touches on touch-sensitive displays also applies to other forms of user inputs to operate multifunction devices 100 with input devices, not all of which are initiated on touch screens. For example, mouse movement and mouse button presses, optionally coordinated with single or multiple keyboard presses or holds; contact movements such as taps, drags, scrolls, etc. on touchpads; pen stylus inputs; movement of the device; oral instructions; detected eye movements; biometric inputs; and/or any combination thereof are optionally utilized as inputs corresponding to sub-events which define an event to be recognized.

FIG. 2 illustrates a portable multifunction device 100 having a touch screen 112 in accordance with some embodiments. The touch screen optionally displays one or more graphics within user interface (UI) 200. In this embodiment, as well as others described below, a user is enabled to select one or more of the graphics by making a gesture on the graphics, for example, with one or more fingers 202 (not drawn to scale in the figure) or one or more styluses 203 (not drawn to scale in the figure). In some embodiments, selection of one or more graphics occurs when the user breaks contact with the one or more graphics. In some embodiments, the gesture optionally includes one or more taps, one or more swipes (from left to right, right to left, upward and/or downward), and/or a rolling of a finger (from right to left, left to right, upward and/or downward) that has made contact with device 100. In some implementations or circumstances, inadvertent contact with a graphic does not select the graphic. For example, a swipe gesture that sweeps over an application icon optionally does not select the corresponding application when the gesture corresponding to selection is a tap.

Device 100 optionally also include one or more physical buttons, such as “home” or menu button 204. As described previously, menu button 204 is, optionally, used to navigate to any application 136 in a set of applications that are, optionally, executed on device 100. Alternatively, in some embodiments, the menu button is implemented as a soft key in a GUI displayed on touch screen 112.

In some embodiments, device 100 includes touch screen 112, menu button 204, push button 206 for powering the device on/off and locking the device, volume adjustment button(s) 208, subscriber identity module (SIM) card slot 210, headset jack 212, and docking/charging external port 124. Push button 206 is, optionally, used to turn the power on/off on the device by depressing the button and holding the button in the depressed state for a predefined time interval; to lock the device by depressing the button and releasing the button before the predefined time interval has elapsed; and/or to unlock the device or initiate an unlock process. In an alternative embodiment, device 100 also accepts verbal input for activation or deactivation of some functions through microphone 113. Device 100 also, optionally, includes one or more contact intensity sensors 165 for detecting intensity of contacts on touch screen 112 and/or one or more tactile output generators 167 for generating tactile outputs for a user of device 100.

FIG. 3A is a block diagram of an exemplary multifunction device with a display and a touch-sensitive surface in accordance with some embodiments. Device 300 need not be portable. In some embodiments, device 300 is a laptop computer, a desktop computer, a tablet computer, a multimedia player device, a navigation device, an educational device (such as a child's learning toy), a gaming system, or a control device (e.g., a home or industrial controller). Device 300 typically includes one or more processing units (CPUs) 310, one or more network or other communications interfaces 360, memory 370, and one or more communication buses 320 for interconnecting these components. Communication buses 320 optionally include circuitry (sometimes called a chipset) that interconnects and controls communications between system components. Device 300 includes input/output (I/O) interface 330 comprising display 340, which is typically a touch screen display. I/O interface 330 also optionally includes a keyboard and/or mouse (or other pointing device) 350 and touchpad 355, tactile output generator 357 for generating tactile outputs on device 300 (e.g., similar to tactile output generator(s) 167 described above with reference to FIG. 1A), sensors 359 (e.g., optical, acceleration, proximity, touch-sensitive, and/or contact intensity sensors similar to contact intensity sensor(s) 165 described above with reference to FIG. 1A). Memory 370 includes high-speed random access memory, such as DRAM, SRAM, DDR RAM, or other random access solid state memory devices; and optionally includes non-volatile memory, such as one or more magnetic disk storage devices, optical disk storage devices, flash memory devices, or other non-volatile solid state storage devices. Memory 370 optionally includes one or more storage devices remotely located from CPU(s) 310. In some embodiments, memory 370 stores programs, modules, and data structures analogous to the programs, modules, and data structures stored in memory 102 of portable multifunction device 100 (FIG. 1A), or a subset thereof. Furthermore, memory 370 optionally stores additional programs, modules, and data structures not present in memory 102 of portable multifunction device 100. For example, memory 370 of device 300 optionally stores drawing module 380, presentation module 382, word processing module 384, website creation module 386, disk authoring module 388, and/or spreadsheet module 390, while memory 102 of portable multifunction device 100 (FIG. 1A) optionally does not store these modules.

Each of the above-identified elements in FIG. 3A is, optionally, stored in one or more of the previously mentioned memory devices. Each of the above-identified modules corresponds to a set of instructions for performing a function described above. The above-identified modules or computer programs (e.g., sets of instructions or including instructions) need not be implemented as separate software programs (such as computer programs (e.g., including instructions)), procedures, or modules, and thus various subsets of these modules are, optionally, combined or otherwise rearranged in various embodiments. In some embodiments, memory 370 optionally stores a subset of the modules and data structures identified above. Furthermore, memory 370 optionally stores additional modules and data structures not described above.

Implementations within the scope of the present disclosure can be partially or entirely realized using a tangible computer-readable storage medium (or multiple tangible computer-readable storage media of one or more types) encoding one or more computer-readable instructions. It should be recognized that computer-readable instructions can be organized in any format, including applications, widgets, processes, software, and/or components.

Implementations within the scope of the present disclosure include a computer-readable storage medium that encodes instructions organized as an application (e.g., application 3160) that, when executed by one or more processing units, control an electronic device (e.g., device 3150) to perform the method of FIG. 3B, the method of FIG. 3C, and/or one or more other processes and/or methods described herein.

It should be recognized that application 3160 (shown in FIG. 3D) can be any suitable type of application, including, for example, one or more of: a browser application, an application that functions as an execution environment for plug-ins, widgets or other applications, a fitness application, a health application, a digital payments application, a media application, a social network application, a messaging application, and/or a maps application. In some embodiments, application 3160 is an application that is pre-installed on device 3150 at purchase (e.g., a first-party application). In some embodiments, application 3160 is an application that is provided to device 3150 via an operating system update file (e.g., a first-party application or a second-party application). In some embodiments, application 3160 is an application that is provided via an application store. In some embodiments, the application store can be an application store that is pre-installed on device 3150 at purchase (e.g., a first-party application store). In some embodiments, the application store is a third-party application store (e.g., an application store that is provided by another application store, downloaded via a network, and/or read from a storage device).

Referring to FIG. 3B and FIG. 3F, application 3160 obtains information (e.g., 3010). In some embodiments, at 3010, information is obtained from at least one hardware component of device 3150. In some embodiments, at 3010, information is obtained from at least one software module of device 3150. In some embodiments, at 3010, information is obtained from at least one hardware component external to device 3150 (e.g., a peripheral device, an accessory device, and/or a server). In some embodiments, the information obtained at 3010 includes positional information, time information, notification information, user information, environment information, electronic device state information, weather information, media information, historical information, event information, hardware information, and/or motion information. In some embodiments, in response to and/or after obtaining the information at 3010, application 3160 provides the information to a system (e.g., 3020).

In some embodiments, the system (e.g., 3110 shown in FIG. 3E) is an operating system hosted on device 3150. In some embodiments, the system (e.g., 3110 shown in FIG. 3E) is an external device (e.g., a server, a peripheral device, an accessory, and/or a personal computing device) that includes an operating system.

Referring to FIG. 3C and FIG. 3G, application 3160 obtains information (e.g., 3030). In some embodiments, the information obtained at 3030 includes positional information, time information, notification information, user information, environment information electronic device state information, weather information, media information, historical information, event information, hardware information, and/or motion information. In response to and/or after obtaining the information at 3030, application 3160 performs an operation with the information (e.g., 3040). In some embodiments, the operation performed at 3040 includes: providing a notification based on the information, sending a message based on the information, displaying the information, controlling a user interface of a fitness application based on the information, controlling a user interface of a health application based on the information, controlling a focus mode based on the information, setting a reminder based on the information, adding a calendar entry based on the information, and/or calling an API of system 3110 based on the information.

In some embodiments, one or more steps of the method of FIG. 3B and/or the method of FIG. 3C is performed in response to a trigger. In some embodiments, the trigger includes detection of an event, a notification received from system 3110, a user input, and/or a response to a call to an API provided by system 3110.

In some embodiments, the instructions of application 3160, when executed, control device 3150 to perform the method of FIG. 3B and/or the method of FIG. 3C by calling an application programming interface (API) (e.g., API 3190) provided by system 3110. In some embodiments, application 3160 performs at least a portion of the method of FIG. 3B and/or the method of FIG. 3C without calling API 3190.

In some embodiments, one or more steps of the method of FIG. 3B and/or the method of FIG. 3C includes calling an API (e.g., API 3190) using one or more parameters defined by the API. In some embodiments, the one or more parameters include a constant, a key, a data structure, an object, an object class, a variable, a data type, a pointer, an array, a list or a pointer to a function or method, and/or another way to reference a data or other item to be passed via the API.

Referring to FIG. 3D, device 3150 is illustrated. In some embodiments, device 3150 is a personal computing device, a smart phone, a smart watch, a fitness tracker, a head mounted display (HM D) device, a media device, a communal device, a speaker, a television, and/or a tablet. As illustrated in FIG. 3D, device 3150 includes application 3160 and an operating system (e.g., system 3110 shown in FIG. 3E). Application 3160 includes application implementation module 3170 and API-calling module 3180. System 3110 includes API 3190 and implementation module 3100. It should be recognized that device 3150, application 3160, and/or system 3110 can include more, fewer, and/or different components than illustrated in FIGS. 3D and 3E.

In some embodiments, application implementation module 3170 includes a set of one or more instructions corresponding to one or more operations performed by application 3160. For example, when application 3160 is a messaging application, application implementation module 3170 can include operations to receive and send messages. In some embodiments, application implementation module 3170 communicates with API-calling module 3180 to communicate with system 3110 via API 3190 (shown in FIG. 3E).

In some embodiments, API 3190 is a software module (e.g., a collection of computer-readable instructions) that provides an interface that allows a different module (e.g., API-calling module 3180) to access and/or use one or more functions, methods, procedures, data structures, classes, and/or other services provided by implementation module 3100 of system 3110. For example, API-calling module 3180 can access a feature of implementation module 3100 through one or more API calls or invocations (e.g., embodied by a function or a method call) exposed by API 3190 (e.g., a software and/or hardware module that can receive API calls, respond to API calls, and/or send API calls) and can pass data and/or control information using one or more parameters via the API calls or invocations. In some embodiments, API 3190 allows application 3160 to use a service provided by a Software Development Kit (SDK) library. In some embodiments, application 3160 incorporates a call to a function or method provided by the SDK library and provided by API 3190 or uses data types or objects defined in the SDK library and provided by API 3190. In some embodiments, API-calling module 3180 makes an API call via API 3190 to access and use a feature of implementation module 3100 that is specified by API 3190. In such embodiments, implementation module 3100 can return a value via API 3190 to API-calling module 3180 in response to the API call. The value can report to application 3160 the capabilities or state of a hardware component of device 3150, including those related to aspects such as input capabilities and state, output capabilities and state, processing capability, power state, storage capacity and state, and/or communications capability. In some embodiments, API 3190 is implemented in part by firmware, microcode, or other low level logic that executes in part on the hardware component.

In some embodiments, API 3190 allows a developer of API-calling module 3180 (which can be a third-party developer) to leverage a feature provided by implementation module 3100. In such embodiments, there can be one or more API-calling modules (e.g., including API-calling module 3180) that communicate with implementation module 3100. In some embodiments, API 3190 allows multiple API-calling modules written in different programming languages to communicate with implementation module 3100 (e.g., API 3190 can include features for translating calls and returns between implementation module 3100 and API-calling module 3180) while API 3190 is implemented in terms of a specific programming language. In some embodiments, API-calling module 3180 calls A Pls from different providers such as a set of A Pls from an OS provider, another set of A Pls from a plug-in provider, and/or another set of A Pls from another provider (e.g., the provider of a software library) or creator of the another set of APIs.

Examples of API 3190 can include one or more of: a pairing API (e.g., for establishing secure connection, e.g., with an accessory), a device detection API (e.g., for locating nearby devices, e.g., media devices and/or smartphone), a payment API, a UIKit API (e.g., for generating user interfaces), a location detection API, a locator API, a maps API, a health sensor API, a sensor API, a messaging API, a push notification API, a streaming API, a collaboration API, a video conferencing API, an application store API, an advertising services API, a web browser API (e.g., WebKit API), a vehicle API, a networking API, a WiFi API, a Bluetooth API, an NFC API, a UWB API, a fitness API, a smart home API, contact transfer API, photos API, camera API, and/or image processing API. In some embodiments, the sensor API is an API for accessing data associated with a sensor of device 3150. For example, the sensor API can provide access to raw sensor data. For another example, the sensor API can provide data derived (and/or generated) from the raw sensor data. In some embodiments, the sensor data includes temperature data, image data, video data, audio data, heart rate data, IM U (inertial measurement unit) data, lidar data, location data, GPS data, and/or camera data. In some embodiments, the sensor includes one or more of an accelerometer, temperature sensor, infrared sensor, optical sensor, heartrate sensor, barometer, gyroscope, proximity sensor, temperature sensor, and/or biometric sensor.

In some embodiments, implementation module 3100 is a system (e.g., operating system and/or server system) software module (e.g., a collection of computer-readable instructions) that is constructed to perform an operation in response to receiving an API call via API 3190. In some embodiments, implementation module 3100 is constructed to provide an API response (via API 3190) as a result of processing an API call. By way of example, implementation module 3100 and API-calling module 3180 can each be any one of an operating system, a library, a device driver, an API, an application program, or other module. It should be understood that implementation module 3100 and API-calling module 3180 can be the same or different type of module from each other. In some embodiments, implementation module 3100 is embodied at least in part in firmware, microcode, or hardware logic.

In some embodiments, implementation module 3100 returns a value through API 3190 in response to an API call from API-calling module 3180. While API 3190 defines the syntax and result of an API call (e.g., how to invoke the API call and what the API call does), API 3190 might not reveal how implementation module 3100 accomplishes the function specified by the API call. Various API calls are transferred via the one or more application programming interfaces between API-calling module 3180 and implementation module 3100. Transferring the API calls can include issuing, initiating, invoking, calling, receiving, returning, and/or responding to the function calls or messages. In other words, transferring can describe actions by either of API-calling module 3180 or implementation module 3100. In some embodiments, a function call or other invocation of API 3190 sends and/or receives one or more parameters through a parameter list or other structure.

In some embodiments, implementation module 3100 provides more than one API, each providing a different view of or with different aspects of functionality implemented by implementation module 3100. For example, one API of implementation module 3100 can provide a first set of functions and can be exposed to third-party developers, and another API of implementation module 3100 can be hidden (e.g., not exposed) and provide a subset of the first set of functions and also provide another set of functions, such as testing or debugging functions which are not in the first set of functions. In some embodiments, implementation module 3100 calls one or more other components via an underlying API and thus is both an API-calling module and an implementation module. It should be recognized that implementation module 3100 can include additional functions, methods, classes, data structures, and/or other features that are not specified through API 3190 and are not available to API-calling module 3180. It should also be recognized that API-calling module 3180 can be on the same system as implementation module 3100 or can be located remotely and access implementation module 3100 using API 3190 over a network. In some embodiments, implementation module 3100, API 3190, and/or API-calling module 3180 is stored in a machine-readable medium, which includes any mechanism for storing information in a form readable by a machine (e.g., a computer or other data processing system). For example, a machine-readable medium can include magnetic disks, optical disks, random access memory; read only memory, and/or flash memory devices.

An application programming interface (API) is an interface between a first software process and a second software process that specifies a format for communication between the first software process and the second software process. Limited APIs (e.g., private A Pls or partner A Pls) are APIs that are accessible to a limited set of software processes (e.g., only software processes within an operating system or only software processes that are approved to access the limited A Pls). Public APIs that are accessible to a wider set of software processes. Some APIs enable software processes to communicate about or set a state of one or more input devices (e.g., one or more touch sensors, proximity sensors, visual sensors, motion/orientation sensors, pressure sensors, intensity sensors, sound sensors, wireless proximity sensors, biometric sensors, buttons, switches, rotatable elements, and/or external controllers). Some APIs enable software processes to communicate about and/or set a state of one or more output generation components (e.g., one or more audio output generation components, one or more display generation components, and/or one or more tactile output generation components). Some A Pls enable particular capabilities (e.g., scrolling, handwriting, text entry, image editing, and/or image creation) to be accessed, performed, and/or used by a software process (e.g., generating outputs for use by a software process based on input from the software process). Some APIs enable content from a software process to be inserted into a template and displayed in a user interface that has a layout and/or behaviors that are specified by the template.

Many software platforms include a set of frameworks that provides the core objects and core behaviors that a software developer needs to build software applications that can be used on the software platform. Software developers use these objects to display content onscreen, to interact with that content, and to manage interactions with the software platform. Software applications rely on the set of frameworks for their basic behavior, and the set of frameworks provides many ways for the software developer to customize the behavior of the application to match the specific needs of the software application. Many of these core objects and core behaviors are accessed via an API. An API will typically specify a format for communication between software processes, including specifying and grouping available variables, functions, and protocols. An API call (sometimes referred to as an API request) will typically be sent from a sending software process to a receiving software process as a way to accomplish one or more of the following: the sending software process requesting information from the receiving software process (e.g., for the sending software process to take action on), the sending software process providing information to the receiving software process (e.g., for the receiving software process to take action on), the sending software process requesting action by the receiving software process, or the sending software process providing information to the receiving software process about action taken by the sending software process. Interaction with a device (e.g., using a user interface) will in some circumstances include the transfer and/or receipt of one or more API calls (e.g., multiple API calls) between multiple different software processes (e.g., different portions of an operating system, an application and an operating system, or different applications) via one or more A Pls (e.g., via multiple different A Pls). For example, when an input is detected the direct sensor data is frequently processed into one or more input events that are provided (e.g., via an API) to a receiving software process that makes some determination based on the input events, and then sends (e.g., via an API) information to a software process to perform an operation (e.g., change a device state and/or user interface) based on the determination. While a determination and an operation performed in response could be made by the same software process, alternatively the determination could be made in a first software process and relayed (e.g., via an API) to a second software process, that is different from the first software process, that causes the operation to be performed by the second software process. Alternatively, the second software process could relay instructions (e.g., via an API) to a third software process that is different from the first software process and/or the second software process to perform the operation. It should be understood that some or all user interactions with a computer system could involve one or more API calls within a step of interacting with the computer system (e.g., between different software components of the computer system or between a software component of the computer system and a software component of one or more remote computer systems). It should be understood that some or all user interactions with a computer system could involve one or more API calls between steps of interacting with the computer system (e.g., between different software components of the computer system or between a software component of the computer system and a software component of one or more remote computer systems).

In some embodiments, the application can be any suitable type of application, including, for example, one or more of: a browser application, an application that functions as an execution environment for plug-ins, widgets or other applications, a fitness application, a health application, a digital payments application, a media application, a social network application, a messaging application, and/or a maps application.

In some embodiments, the application is an application that is pre-installed on the first computer system at purchase (e.g., a first-party application). In some embodiments, the application is an application that is provided to the first computer system via an operating system update file (e.g., a first-party application). In some embodiments, the application is an application that is provided via an application store. In some embodiments, the application store is pre-installed on the first computer system at purchase (e.g., a first-party application store) and allows download of one or more applications. In some embodiments, the application store is a third-party application store (e.g., an application store that is provided by another device, downloaded via a network, and/or read from a storage device). In some embodiments, the application is a third-party application (e.g., an app that is provided by an application store, downloaded via a network, and/or read from a storage device). In some embodiments, the application controls the first computer system to perform method 700 and/or method 800 (FIG. 7 and FIG. 8) by calling an application programming interface (API) provided by the system process using one or more parameters.

In some embodiments, exemplary APIs provided by the system process include one or more of: a pairing API (e.g., for establishing secure connection, e.g., with an accessory), a device detection API (e.g., for locating nearby devices, e.g., media devices and/or smartphone), a payment API, a UIKit API (e.g., for generating user interfaces), a location detection API, a locator API, a maps API, a health sensor API, a sensor API, a messaging API, a push notification API, a streaming API, a collaboration API, a video conferencing API, an application store API, an advertising services API, a web browser API (e.g., WebKit API), a vehicle API, a networking API, a WiFi API, a Bluetooth API, an NFC API, a UWB API, a fitness API, a smart home API, contact transfer API, a photos API, a camera API, and/or an image processing API.

In some embodiments, at least one API is a software module (e.g., a collection of computer-readable instructions) that provides an interface that allows a different module (e.g., API-calling module 3180) to access and use one or more functions, methods, procedures, data structures, classes, and/or other services provided by an implementation module of the system process. The API can define one or more parameters that are passed between the API-calling module and the implementation module. In some embodiments, API 3190 defines a first API call that can be provided by API-calling module 3180. The implementation module is a system software module (e.g., a collection of computer-readable instructions) that is constructed to perform an operation in response to receiving an API call via the API. In some embodiments, the implementation module is constructed to provide an API response (via the API) as a result of processing an API call. In some embodiments, the implementation module is included in the device (e.g., 3150) that runs the application. In some embodiments, the implementation module is included in an electronic device that is separate from the device that runs the application.

Attention is now directed towards embodiments of user interfaces that are, optionally, implemented on, for example, portable multifunction device 100.

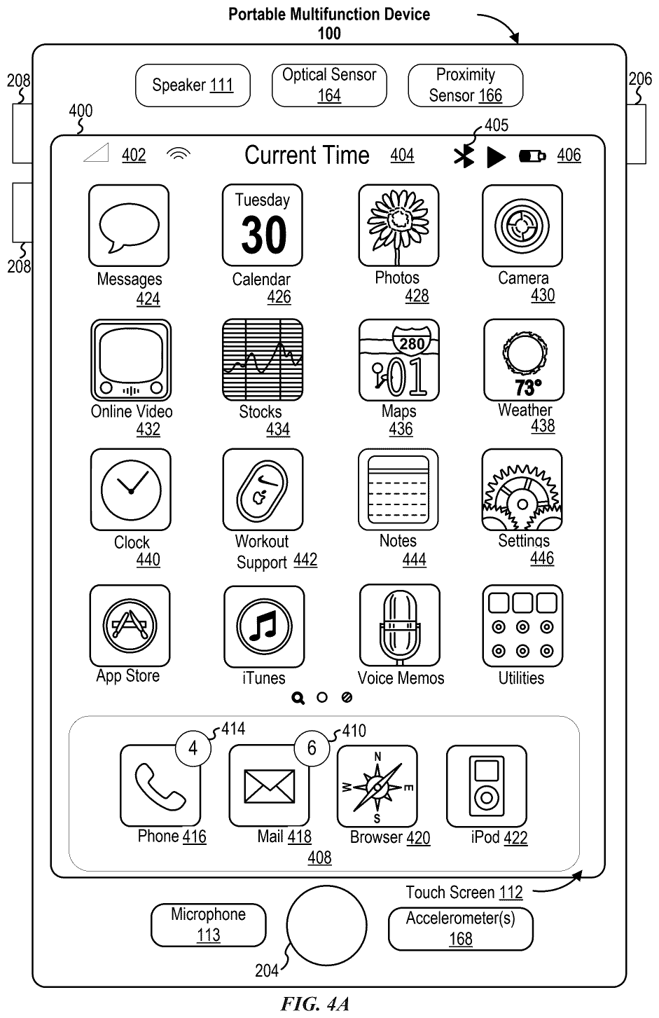

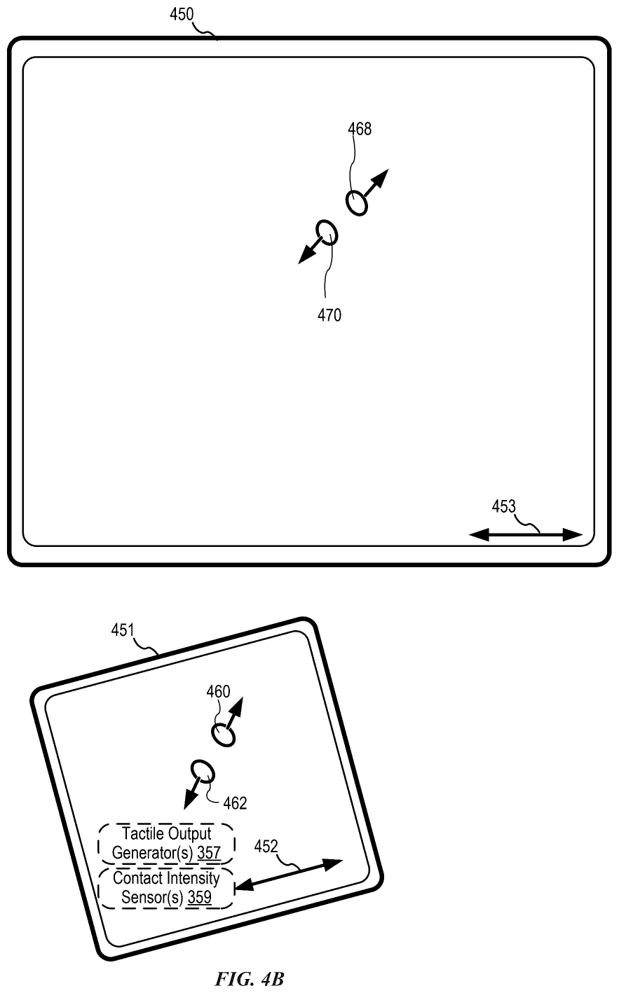

FIG. 4A illustrates an exemplary user interface for a menu of applications on portable multifunction device 100 in accordance with some embodiments. Similar user interfaces are, optionally, implemented on device 300. In some embodiments, user interface 400 includes the following elements, or a subset or superset thereof:
Signal strength indicator(s) 402 for wireless communication(s), such as cellular and Wi-Fi signals;
Time 404;
Bluetooth indicator 405;
Battery status indicator 406;
Tray 408 with icons for frequently used applications, such as:
   Icon 416 for telephone module 138, labeled “Phone,” which optionally includes an indicator 414 of the number of missed calls or voicemail messages;
   Icon 418 for e-mail client module 140, labeled “Mail,” which optionally includes an indicator 410 of the number of unread e-mails;
   Icon 420 for browser module 147, labeled “Browser;” and
   Icon 422 for video and music player module 152, also referred to as iPod (trademark of Apple Inc.) module 152, labeled “iPod;” and
Icons for other applications, such as:
   Icon 424 for IM module 141, labeled “Messages;”
   Icon 426 for calendar module 148, labeled “Calendar;”
   Icon 428 for image management module 144, labeled “Photos;”
   Icon 430 for camera module 143, labeled “Camera;”
   Icon 432 for online video module 155, labeled “Online Video;”
   Icon 434 for stocks widget 149-2, labeled “Stocks;”
   Icon 436 for map module 154, labeled “Maps;”
   Icon 438 for weather widget 149-1, labeled “Weather;”
   Icon 440 for alarm clock widget 149-4, labeled “Clock;”
   Icon 442 for workout support module 142, labeled “Workout Support;”
   Icon 444 for notes module 153, labeled “Notes;” and
   Icon 446 for a settings application or module, labeled “Settings,” which provides access to settings for device 100 and its various applications 136.

It should be noted that the icon labels illustrated in FIG. 4A are merely exemplary. For example, icon 422 for video and music player module 152 is labeled “Music” or “Music Player.” Other labels are, optionally, used for various application icons. In some embodiments, a label for a respective application icon includes a name of an application corresponding to the respective application icon. In some embodiments, a label for a particular application icon is distinct from a name of an application corresponding to the particular application icon.

FIG. 4B illustrates an exemplary user interface on a device (e.g., device 300, FIG. 3A) with a touch-sensitive surface 451 (e.g., a tablet or touchpad 355, FIG. 3A) that is separate from the display 450 (e.g., touch screen display 112). Device 300 also, optionally, includes one or more contact intensity sensors (e.g., one or more of sensors 359) for detecting intensity of contacts on touch-sensitive surface 451 and/or one or more tactile output generators 357 for generating tactile outputs for a user of device 300.

Although some of the examples that follow will be given with reference to inputs on touch screen display 112 (where the touch-sensitive surface and the display are combined), in some embodiments, the device detects inputs on a touch-sensitive surface that is separate from the display, as shown in FIG. 4B. In some embodiments, the touch-sensitive surface (e.g., touch sensitive surface 451 in FIG. 4B) has a primary axis (e.g., 452 in FIG. 4B) that corresponds to a primary axis (e.g., 453 in FIG. 4B) on the display (e.g., display 450). In accordance with these embodiments, the device detects contacts (e.g., contact 460 and contact 462 in FIG. 4B) with the touch-sensitive surface 451 at locations that correspond to respective locations on the display (e.g., in FIG. 4B, contact 460 corresponds to 468 and contact 462 corresponds to 470). In this way, user inputs (e.g., contacts 460 and 462, and movements thereof) detected by the device on the touch-sensitive surface (e.g., touch sensitive surface 451 in FIG. 4B) are used by the device to manipulate the user interface on the display (e.g., display 450 in FIG. 4B) of the multifunction device when the touch-sensitive surface is separate from the display. It should be understood that similar methods are, optionally, used for other user interfaces described herein.

Additionally, while the following examples are given primarily with reference to finger inputs (e.g., finger contacts, finger tap gestures, finger swipe gestures), it should be understood that, in some embodiments, one or more of the finger inputs are replaced with input from another input device (e.g., a mouse-based input or stylus input). For example, a swipe gesture is, optionally, replaced with a mouse click (e.g., instead of a contact) followed by movement of the cursor along the path of the swipe (e.g., instead of movement of the contact). As another example, a tap gesture is, optionally, replaced with a mouse click while the cursor is located over the location of the tap gesture (e.g., instead of detection of the contact followed by ceasing to detect the contact). Similarly, when multiple user inputs are simultaneously detected, it should be understood that multiple computer mice are, optionally, used simultaneously, or a mouse and finger contacts are, optionally, used simultaneously.

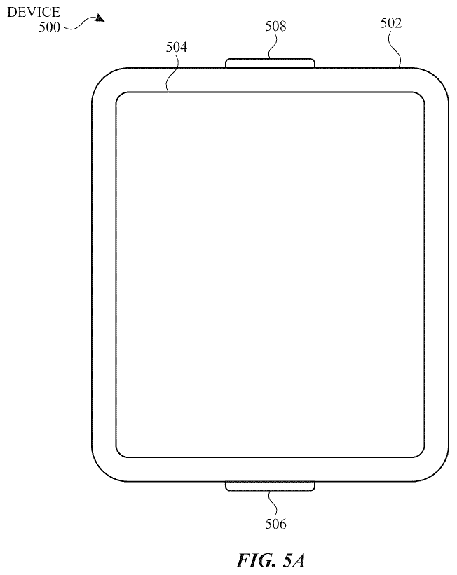

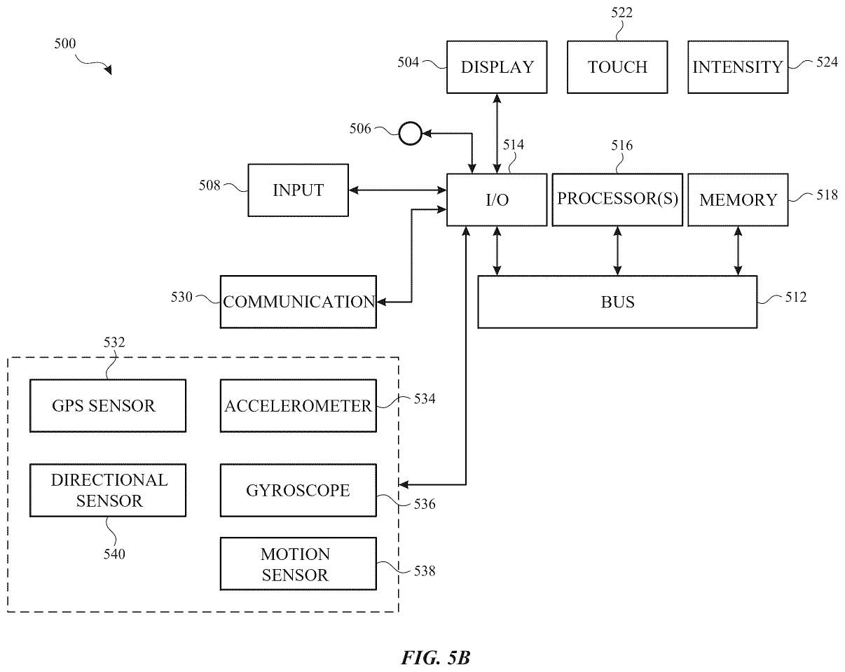

FIG. 5A illustrates exemplary personal electronic device 500. Device 500 includes body 502. In some embodiments, device 500 can include some or all of the features described with respect to devices 100 and 300 (e.g., FIGS. 1A-4B). In some embodiments, device 500 has touch-sensitive display screen 504, hereafter touch screen 504. Alternatively, or in addition to touch screen 504, device 500 has a display and a touch-sensitive surface. As with devices 100 and 300, in some embodiments, touch screen 504 (or the touch-sensitive surface) optionally includes one or more intensity sensors for detecting intensity of contacts (e.g., touches) being applied. The one or more intensity sensors of touch screen 504 (or the touch-sensitive surface) can provide output data that represents the intensity of touches. The user interface of device 500 can respond to touches based on their intensity, meaning that touches of different intensities can invoke different user interface operations on device 500.

Exemplary techniques for detecting and processing touch intensity are found, for example, in related applications: International Patent Application Serial No. PCT/US2013/040061, titled “Device, Method, and Graphical User Interface for Displaying User Interface Objects Corresponding to an Application,” filed May 8, 2013, published as WIPO Publication No. WO/2013/169849, and International Patent Application Serial No. PCT/US2013/069483, titled “Device, Method, and Graphical User Interface for Transitioning Between Touch Input to Display Output Relationships,” filed Nov. 11, 2013, published as WIPO Publication No. WO/2014/105276, each of which is hereby incorporated by reference in their entirety.

In some embodiments, device 500 has one or more input mechanisms 506 and 508. Input mechanisms 506 and 508, if included, can be physical. Examples of physical input mechanisms include push buttons and rotatable mechanisms. In some embodiments, device 500 has one or more attachment mechanisms. Such attachment mechanisms, if included, can permit attachment of device 500 with, for example, hats, eyewear, earrings, necklaces, shirts, jackets, bracelets, watch straps, chains, trousers, belts, shoes, purses, backpacks, and so forth. These attachment mechanisms permit device 500 to be worn by a user.

FIG. 5B depicts exemplary personal electronic device 500. In some embodiments, device 500 can include some or all of the components described with respect to FIGS. 1A, 1B, and 3A. Device 500 has bus 512 that operatively couples I/O section 514 with one or more computer processors 516 and memory 518. I/O section 514 can be connected to display screen 504, which can have touch-sensitive component 522 and, optionally, intensity sensor 524 (e.g., contact intensity sensor). In addition, I/O section 514 can be connected with communication unit 530 for receiving application and operating system data, using Wi-Fi, Bluetooth, near field communication (NFC), cellular, and/or other wireless communication techniques. Device 500 can include input mechanisms 506 and/or 508. Input mechanism 506 is, optionally, a rotatable input device or a depressible and rotatable input device, for example. Input mechanism 508 is, optionally, a button, in some examples.

Input mechanism 508 is, optionally, a microphone, in some examples. Personal electronic device 500 optionally includes various sensors, such as GPS sensor 532, accelerometer 534, directional sensor 540 (e.g., compass), gyroscope 536, motion sensor 538, and/or a combination thereof, all of which can be operatively connected to I/O section 514.

Memory 518 of personal electronic device 500 can include one or more non-transitory computer-readable storage mediums, for storing computer-executable instructions, which, when executed by one or more computer processors 516, for example, can cause the computer processors to perform the techniques described below, including processes 700-800 (FIGS. 7-8). A computer-readable storage medium can be any medium that can tangibly contain or store computer-executable instructions for use by or in connection with the instruction execution system, apparatus, or device. In some examples, the storage medium is a transitory computer-readable storage medium. In some examples, the storage medium is a non-transitory computer-readable storage medium. The non-transitory computer-readable storage medium can include, but is not limited to, magnetic, optical, and/or semiconductor storages. Examples of such storage include magnetic disks, optical discs based on CD, DVD, or Blu-ray® technologies, as well as persistent solid-state memory such as flash, solid-state drives, and the like. Personal electronic device 500 is not limited to the components and configuration of FIG. 5B, but can include other or additional components in multiple configurations.

As used here, the term “affordance” refers to a user-interactive graphical user interface object that is, optionally, displayed on the display screen of devices 100, 300, and/or 500 (FIGS. 1A, 3A, and 5A-5B). For example, an image (e.g., icon), a button, and text (e.g., hyperlink) each optionally constitute an affordance.

As used herein, the term “focus selector” refers to an input element that indicates a current part of a user interface with which a user is interacting. In some implementations that include a cursor or other location marker, the cursor acts as a “focus selector” so that when an input (e.g., a press input) is detected on a touch-sensitive surface (e.g., touchpad 355 in FIG. 3A or touch-sensitive surface 451 in FIG. 4B) while the cursor is over a particular user interface element (e.g., a button, window, slider, or other user interface element), the particular user interface element is adjusted in accordance with the detected input. In some implementations that include a touch screen display (e.g., touch-sensitive display system 112 in FIG. 1A or touch screen 112 in FIG. 4A) that enables direct interaction with user interface elements on the touch screen display, a detected contact on the touch screen acts as a “focus selector” so that when an input (e.g., a press input by the contact) is detected on the touch screen display at a location of a particular user interface element (e.g., a button, window, slider, or other user interface element), the particular user interface element is adjusted in accordance with the detected input. In some implementations, focus is moved from one region of a user interface to another region of the user interface without corresponding movement of a cursor or movement of a contact on a touch screen display (e.g., by using a tab key or arrow keys to move focus from one button to another button); in these implementations, the focus selector moves in accordance with movement of focus between different regions of the user interface. Without regard to the specific form taken by the focus selector, the focus selector is generally the user interface element (or contact on a touch screen display) that is controlled by the user so as to communicate the user's intended interaction with the user interface (e.g., by indicating, to the device, the element of the user interface with which the user is intending to interact). For example, the location of a focus selector (e.g., a cursor, a contact, or a selection box) over a respective button while a press input is detected on the touch-sensitive surface (e.g., a touchpad or touch screen) will indicate that the user is intending to activate the respective button (as opposed to other user interface elements shown on a display of the device).

As used in the specification and claims, the term “characteristic intensity” of a contact refers to a characteristic of the contact based on one or more intensities of the contact. In some embodiments, the characteristic intensity is based on multiple intensity samples. The characteristic intensity is, optionally, based on a predefined number of intensity samples, or a set of intensity samples collected during a predetermined time period (e.g., 0.05, 0.1, 0.2, 0.5, 1, 2, 5, 10 seconds) relative to a predefined event (e.g., after detecting the contact, prior to detecting liftoff of the contact, before or after detecting a start of movement of the contact, prior to detecting an end of the contact, before or after detecting an increase in intensity of the contact, and/or before or after detecting a decrease in intensity of the contact). A characteristic intensity of a contact is, optionally, based on one or more of: a maximum value of the intensities of the contact, a mean value of the intensities of the contact, an average value of the intensities of the contact, a top 10 percentile value of the intensities of the contact, a value at the half maximum of the intensities of the contact, a value at the 90 percent maximum of the intensities of the contact, or the like. In some embodiments, the duration of the contact is used in determining the characteristic intensity (e.g., when the characteristic intensity is an average of the intensity of the contact over time). In some embodiments, the characteristic intensity is compared to a set of one or more intensity thresholds to determine whether an operation has been performed by a user. For example, the set of one or more intensity thresholds optionally includes a first intensity threshold and a second intensity threshold. In this example, a contact with a characteristic intensity that does not exceed the first threshold results in a first operation, a contact with a characteristic intensity that exceeds the first intensity threshold and does not exceed the second intensity threshold results in a second operation, and a contact with a characteristic intensity that exceeds the second threshold results in a third operation. In some embodiments, a comparison between the characteristic intensity and one or more thresholds is used to determine whether or not to perform one or more operations (e.g., whether to perform a respective operation or forgo performing the respective operation), rather than being used to determine whether to perform a first operation or a second operation.

As used herein, an “installed application” refers to a software application that has been downloaded onto an electronic device (e.g., devices 100, 300, and/or 500) and is ready to be launched (e.g., become opened) on the device. In some embodiments, a downloaded application becomes an installed application by way of an installation program that extracts program portions from a downloaded package and integrates the extracted portions with the operating system of the computer system.

As used herein, the terms “open application” or “executing application” refer to a software application with retained state information (e.g., as part of device/global internal state 157 and/or application internal state 192). An open or executing application is, optionally, any one of the following types of applications:
an active application, which is currently displayed on a display screen of the device that the application is being used on;
a background application (or background processes), which is not currently displayed, but one or more processes for the application are being processed by one or more processors; and
a suspended or hibernated application, which is not running, but has state information that is stored in memory (volatile and non-volatile, respectively) and that can be used to resume execution of the application.

As used herein, the term “closed application” refers to software applications without retained state information (e.g., state information for closed applications is not stored in a memory of the device). Accordingly, closing an application includes stopping and/or removing application processes for the application and removing state information for the application from the memory of the device. Generally, opening a second application while in a first application does not close the first application. When the second application is displayed and the first application ceases to be displayed, the first application becomes a background application.

In some embodiments, the computer system is in a locked state or an unlocked state. In the locked state, the computer system is powered on and operational but is prevented from performing a predefined set of operations in response to user input. The predefined set of operations optionally includes navigation between user interfaces, activation or deactivation of a predefined set of functions, and activation or deactivation of certain applications. The locked state can be used to prevent unintentional or unauthorized use of some functionality of the computer system or activation or deactivation of some functions on the computer system. In some embodiments, in the unlocked state, the computer system is powered on and operational and is not prevented from performing at least a portion of the predefined set of operations that cannot be performed while in the locked state. When the computer system is in the locked state, the computer system is said to be locked. When the computer system is in the unlocked state, the computer is said to be unlocked. In some embodiments, the computer system in the locked state optionally responds to a limited set of user inputs, including input that corresponds to an attempt to transition the computer system to the unlocked state or input that corresponds to powering the computer system off.

Attention is now directed towards embodiments of user interfaces (“UI”) and associated processes that are implemented on an electronic device, such as portable multifunction device 100, device 300, or device 500.

FIGS. 6A-6A C illustrate exemplary user interfaces for managing workout-based notifications, in accordance with some embodiments. The user interfaces in these figures are used to illustrate the processes described below, including the processes in FIG. 7 and FIG. 8.

FIG. 6A illustrates computer system 600, which is a smart watch with touch-sensitive display 602 and rotatable and depressible input mechanism 604 (e.g., a rotatable and depressible crown). At FIG. 6A, computer system 600 displays user interface 606. User interface 606 is a workout selection user interface that allows the user of computer system 600 to initiate a workout session corresponding to a selected workout type (e.g., workout modality). User interface 606 includes platter 606a corresponding to a first workout type (e.g., outdoor run) and platter 606b corresponding to a second workout type (e.g., outdoor walk). At FIG. 6A, computer system 600 detects user input 607, which is a touch input (e.g., a tap input) corresponding to selection of platter 606a.

At FIG. 6B, in response to user input 607, computer system 600 initiates an outdoor run workout session, and displays user interface 608. User interface 608 corresponds to and/or is indicative of an active workout session, and includes modality indication 608a and workout session metrics 608b. Modality indication 608a indicates the workout type of the workout session (e.g., a running workout in FIG. 6B). Workout session metrics 608b include one or more metrics corresponding to the active workout session. For example, in FIG. 6B, workout session metrics 608b include (e.g., from top down) the duration of the workout session (e.g., how long the workout session has been going); a physical activity zone for the user that is indicative of the intensity of the user's physical activity; heart rate; time in the current physical activity zone; and maximum heart rate during the current workout session. At FIG. 6B, computer system 600 detects user input 609, which is a swipe right touch input.

At FIG. 6C, in response to user input 609, computer system 600 displays user interface 610. User interface 610 includes options 610a-610e. Option 610a, when selected, causes computer system 600 to end the workout session. Option 610b, when selected, causes computer system 600 to pause the workout session. Option 610c, when selected, causes computer system 600 to initiate a process for starting a new workout session of a different workout type. Option 610d, when selected, causes computer system 600 to mark a new segment in the current workout session. Option 610e, when selected, causes computer system 600 to initiate a process for creating a new workout-based check-in session with an external computer system and/or a remote user. In some embodiments, when a workout-based check-in session between computer system 600 and an external computer system (and/or between the user of computer system 600 and a remote user) is active, computer system 600 automatically transmits notifications to the external computer system when one or more notification criteria are met. For example, in some embodiments, when computer system 600 detects that the workout session of the user of computer system 600 is interrupted (e.g., based on movement information collected from the user of computer system 600 and/or based on movement information collection by computer system 600), which may indicate that the user of computer system 600 requires assistance, computer system 600 automatically transmits a notification to the external computer system. Various embodiments of such features and workout-based check-in sessions and corresponding notifications are described in greater detail below. At FIG. 6C, computer system 600 detects user input 611, which is a touch input (e.g., a tap input) corresponding to selection of option 610e.

At FIG. 6D, in response to user input 611, computer system 600 displays user interface 612. In some embodiments, user interface 612 is a contact selection user interface that allows the user of computer system 600 to identify one or more contacts with whom the user would like to initiate a workout-based check-in session. User interface 612 includes contact options 614a-614d corresponding to different contacts (e.g., different contacts stored on computer system 600 and/or saved to a contact list). At FIG. 6D, computer system 600 detects user input 615, which is a touch input (e.g., a tap input) corresponding to selection of contact option 614a (e.g., a contact named Ivy).

At FIG. 6E, in response to user input 615, computer system 600 displays user interface 616. User interface 616 indicates that the selected contact, Ivy, will be notified when: (1) the user of computer system 600 provides a user input requesting transmission of a check-in notification to Ivy; (2) the user of computer system 600 ends the workout session; (3) the user of computer system 600 does not respond to a prompt output by computer system 600; and (4) computer system 600 and/or another computer system corresponding to the user of computer system 600 (e.g., the user's phone) are disconnected from a communications network and/or offline. User interface 616 includes options 616a and 616b. Option 616a, when selected, causes computer system 600 to return to the state shown in FIG. 6D. Option 616b, when selected, causes computer system 600 to proceed and/or progress in the process for initiating a workout-based check-in session with the selected contact. At FIG. 6E, computer system 600 detects user input 617 (e.g., a tap input) corresponding to selection of option 616b.

At FIG. 6F, in response to user input 617, computer system 600 displays user interface 618. User interface 618 includes options 620a-620b. Option 620b, when selected, corresponds to a user request to transmit a “full” set of user details when check-in notifications are sent to Ivy. For example, in some embodiments, selecting option 620b causes check-in notifications to Ivy to include the user's location information. Option 620a, when selected, corresponds to a user request to transmit a more limited set of user details when check-in notifications are sent to Ivy that includes less information than had option 620b been selected. For example, in some embodiments, selecting option 620a causes check-in notification to Ivy to exclude the user's location information. At FIG. 6F, computer system 600 detects user input 621a (e.g., a tap input) corresponding to selection of option 620b.

At FIG. 6G, in response to user input 621a, computer system 600 displays selection indication 620b-1 in option 620b to indicate that option 620b has been selected. User interface 618 also includes options 618a and 618b. Option 618a, when selected, causes computer system 600 to return to displaying user interface 616, as in FIG. 6E. Option 618b, when selected, causes computer system 600 to proceed and/or progress in the process for initiating a workout-based check-in session with the selected contact. At FIG. 6G, computer system 600 detects user input 621b (e.g., a tap input) corresponding to selection of option 618b.

At FIG. 6H, in response to user input 621b, computer system 600 displays user interface 622. User interface 622 provides a summary of the workout-based check-in session that the user is in the process of initiating. User interface 622 includes platters 624a-624c. Platter 624a indicates that the user is initiating a workout-based check-in session that will automatically transmit a check-in notification to Ivy when the user chooses to end the workout session and, in some embodiments, is initiating a workout-based check-in session that will automatically terminate and/or end when the user ends the workout session. Platter 624b indicates that the user has opted to share “full” check-in details with Ivy (e.g., including displaying the user's location when a check-in notification is sent to Ivy). Platter 624c, when selected, causes computer system 600 to display a user interface that explains when check-in notifications are sent to Ivy during the workout-based check-in session (e.g., a user interface similar to user interface 616 of FIG. 6E). User interface 622 includes options 622a-622b. Option 622a, when selected, cancels the request to initiate the workout-based check-in session (e.g., without starting and/or initiating the workout-based check-in session). Option 622b, when selected, causes computer system 600 to initiate the workout-based check-in session. At FIG. 6H, computer system 600 detects user input 625 (e.g., a tap input) corresponding to selection of option 622b.

At FIG. 6I, in response to user input 625, computer system 600 initiates the workout-based check-in session with remote user Ivy, and displays user interface 608. In FIG. 6I, user interface 608 is now displayed with indication 626, which indicates that workout-based check-in session with a remote user is now active. Additionally, in response to user input 625, computer system 600 transmits information to one or more external computer systems associated with the remote user Ivy to notify Ivy that the user of computer system 600 has initiated a workout-based check-in session with the remote user. In FIG. 6I, the remote user Ivy is associated with two computer systems 630, 640. Computer system 630 is Ivy's smart watch with touch-sensitive display 632 and rotatable and depressible input mechanism 634, and computer system 640 is Ivy's smart phone with touch-sensitive display 642. The information transmitted by computer system 600 causes computer system 632 to display notification 636, and causes computer system 640 to display notification 648. Notification 636 includes option 636a, information 636b, and information 636c. Information 636b notifies Ivy that Ivy will be notified when the user of computer system 600 (named Jill) ends their workout, and that Ivy will also be notified if Jill's workout is interrupted or if Jill initiates an emergency SOS call (e.g., to first responders or emergency response services). Information 636b also informs Ivy that, in such scenarios, Ivy will be notified with Jill's location. Information 636c indicates Jill (the user of computer system 600) started an outdoor run workout at 9:41 PM. On computer system 640 (e.g., Ivy's phone), notification 648 is displayed as a notification within lock screen user interface 644, which is indicative of computer system 640 being in a locked state. At FIG. 6I, computer system 630 detects user input 647a (e.g., a tap input) corresponding to selection of option 636a, and computer system 640 detects user input 647b (e.g., a tap input) corresponding to selection of notification 648.

At FIG. 6J, in response to user input 647a, computer system 630 ceases display of notification 636 and displays messaging user interface 648. In some embodiments, messaging user interface 648 corresponds to a messaging session between Jill (the user of computer system 600) and Ivy (the user of computer system 630) (e.g., in some embodiments, an already existing messaging session between the two users). Messaging session user interface 648 displays a new message, message 649a, which indicates that Jill has initiated a workout-based check-in session with Ivy. In some embodiments, messaging session user interface 648 also displays additional and/or previous messages that were transmitting into the messaging session by Ivy or Jill prior to message 649a. In some embodiments, message 649a, when selected, causes computer system 630 to display additional details about the workout-based check-in session (e.g., a user interface that includes the same information as and/or at least some of the information shown in notification 636).

At FIG. 6J, in response to user input 647b, computer system 640 displays messaging user interface 650. In some embodiments, messaging user interface 650 also corresponds to a messaging session between Jill and Ivy. Messaging session user interface 650 includes participant indication 650a, which indicates that the messaging session is a session between Ivy (e.g., the user of computer system 640) and Jill; transmitted messages region 650d that includes messages 652a-652b that have been transmitted into the messaging session; text input field 650b, and keyboard 650c. In FIG. 6J, messaging session user interface 650 displays message 652b, which indicates that Jill has initiated a workout-based check-in session with Ivy. In some embodiments, message 652b, when selected, causes computer system 640 to display additional details about the workout-based check-in session (e.g., a user interface that includes the same information as and/or at least some of the information shown in notification 636).

At FIG. 6K, the user of computer system 600 has progressed in the workout, and computer system 600 displays user interface 608. User interface 608 is displayed with indication 626 indicating that the workout-based check-in session with Ivy is still active.

At FIG. 6L, computer system 600 detects that one or more alert criteria have been satisfied for prompting the user of computer system 600, and displays alert 654. In some embodiments, the one or more alert criteria are met based on movement information corresponding to computer system 600 and/or the user of computer system 600. For example, in some embodiments, the one or more alert criteria are met when the movement of computer system 600 and/or the movement of the user of computer system 600 indicate that the user's workout has been interrupted. In some embodiments, the one or more alert criteria are met when the user's movement and/or the movement of computer system 600 drops below a threshold level of movement and/or drops below a threshold speed of movement (e.g., indicating that the user is no longer moving and/or is no longer running). In some embodiments, the one or more alert criteria are met when the user's movement and/or the movement of computer system 600 is above a threshold speed (e.g., indicating that the user is in a motor vehicle).

Alert 654 includes information 654e which indicates that computer system 600 has detected that the user's workout may have been interrupted, and that Ivy will be notified if the user of computer system 600 does not interact with alert 654 within fifteen minutes. In some embodiments, information 654e is updated over time to indicate how much time is left for the user to interact with alert 654 (e.g., the text “15 minutes” changes to “14 minutes” and “13 minutes” and so forth as time progresses without the user of computer system 600 interacting with alert 654).

Alert 654 includes options 654a, 654b, 654c and 654d. Option 654a, when selected, causes computer system 600 to cease display of alert 654 without sending a check-in notification to remote user Ivy. For example, in some embodiments, selection of option 654a corresponds to an indication that the user of computer system 600 is not in danger and/or is not in need of assistance. Option 654b, when selected, causes computer system 600 to terminate the workout-based check-in session with remote user Ivy, and to send a notification to remote user Ivy that the workout-based check-in session has been ended. Once the workout-based check-in session is terminated, computer system 600 no longer sends automatic notifications to remote user Ivy when notification criteria are satisfied (e.g., no longer sends automatic notifications to Ivy when the user of computer system 600 initiates an emergency SOS call and/or when computer system 600 is disconnected from a communications network); and computer system 600 also no longer displays prompt 654 when alert notification criteria are satisfied (e.g., no longer displays prompt 654 based on the movement of computer system 600 and/or the user of computer system 600 indicating that the user's workout has been interrupted). Option 654c, when selected, is indicative of a user request for computer system 600 to wait additional time before sending a check-in notification to remote user Ivy and/or indicative of a user request to give the user more time to interact with alert 654 (e.g., give the user more time to select option 654a). Option 654d, when selected, causes computer system 600 to send a check-in notification to remote user Ivy (e.g., even before the 15 minutes have elapsed). FIG. 6L depicts three example scenarios in which computer system 600 detects three different user inputs: user input 656a corresponding to selection of option 654c, user input 656b corresponding to selection of option 654b, and user input 656c corresponding to selection of option 654d. Each of these user inputs and scenarios is described below.

At FIG. 6M, in response to user input 654a in FIG. 6L, computer system 600 displays user interface 658. User interface 658 includes options 658a-658d. Option 658a, when selected, causes computer system 600 to cease display of user interface 658 and re-display user interface 654 without adding additional time before a check-in notification is sent to remote user Ivy. Option 658b, when selected, causes computer system 600 to add 15 minutes before a check-in notification is sent to remote user Ivy and, in some embodiments, causes computer system 600 to re-display user interface 654 with information 654e updated to indicate the added time. Option 658c, when selected, causes computer system 600 to add 30 minutes before a check-in notification is sent to remote user Ivy and, in some embodiments, causes computer system 600 to re-display user interface 654 with information 654e updated to indicate the added time. Option 658d, when selected, causes computer system 600 to add 60 minutes before a check-in notification is sent to remote user Ivy and, in some embodiments, causes computer system 600 to re-display user interface 654 with information 654e updated to indicate the added time.

At FIG. 6N, in response to user input 654b in FIG. 6L, computer system 600 displays notification 662 indicating that the workout-based check-in session has been ended. In FIG. 6L, notification 662 is displayed within time user interface 660, which includes current date indication 660a and current time indication 660b. In some embodiments, notification 662 is displayed concurrently with and/or within workout session user interface 608. At FIG. 6N, in response to and/or based on user input 654b in FIG. 6L, computer system 600 also transmits information to computer system 630 (which is associated with remote user Ivy) indicating that the user of computer system 600 has ended the workout-based check-in session. In response to receiving this information from computer system 600, computer system 630 displays notification 664. Notification 664 includes options 664a-664c, map 664d, and information 664e-664f. Option 664a, when selected, causes computer system 630 to cease display of notification 664. Option 664b, when selected, causes computer system 630 to initiate a telephone call and/or an audio call with Jill, the user of computer system 600. Option 664c, when selected, causes computer system 630 to display the current location of Jill, the user of computer system 600, and/or the current location of computer system 600. In some embodiments, map 664d displays the current location of Jill and/or computer system 600. In some embodiments, map 664d displays the location of Jill and/or computer system 600 when the workout-based check-in session was terminated. Information 664e indicates that Jill, the user of computer system 600, started their outdoor run workout at 9:41 PM, and information 664f indicates that Jill ended the workout-based check-in session at 10:09 PM.

At FIG. 6O, in response to user input 654c in FIG. 6L and/or in response to a determination that the user of computer system 600 has not interacted with alert 654 (e.g., has not selected option 654a) within the threshold amount of time, computer system 600 displays user interface 666, which indicates that a check-in notification has been sent to remote user Ivy, and that the location of computer system 600 is being shared with remote user Ivy. User interface 666 includes option 666a and option 666c. Option 666a, when selected, causes computer system 600 to cease display of user interface 666. Option 666c, when selected, causes computer system 600 to stop sharing location information with remote user Ivy.

At FIG. 6O, in response to user input 654c in FIG. 6L and/or in response to a determination that the user of computer system 600 has not interacted with alert 654 (e.g., has not selected option 654a) within the threshold amount of time, computer system 600 also transmits to computer system 630 (e.g., a computer system associated with remote user Ivy) check-in notification information. In response to receiving this information from computer system 600, computer system 630 displays notification 668. Notification 668 includes options 668a-668c, map 668d, and information 668e-668f. Option 668a, when selected, causes computer system 630 to cease display of notification 668. Option 668b, when selected, causes computer system 630 to initiate a telephone call and/or an audio call with Jill, the user of computer system 600. Option 668c, when selected, causes computer system 630 to display detailed map 670 in FIG. 6P. In some embodiments, map 668d displays the current location of Jill and/or computer system 600. Information 664e indicates that Jill, the user of computer system 600, started their outdoor run workout at 9:41 PM. Information 664f indicates that J ill's workout was interrupted and/or a check-in notification was transmitted at 10:09 PM.

In FIG. 6O, notification 668 includes location information corresponding to computer system 600 and/or the user of computer system 600 because the user of computer system 600 opted to share “full” details with remote user Ivy. In some embodiments, had the user of computer system 600 opted to share “limited” details with remote user Ivy, notification 668 would not include option 668c and/or map 668d, as shown in FIG. 6O1.

At FIG. 6O, computer system 630 detects user input 669, which is a touch input corresponding to selection of map 668d. At FIG. 6P, in response to user input 669, computer system 630 displays map 670, which provides more detailed information about the current location of computer system 600 and/or the user of computer system 600. For example, in FIG. 6P, map 670 displays workout start location indication 670d indicating the location at which Jill started her workout, workout interruption indication 670e indicating the location of computer system 600 when the check-in notification was transmitted at 10:09 PM, route indication 670c indicating the route of computer system 600 and/or the user of computer system 600, and current location indication 670b indicating the current location of computer system 600 and/or the user of computer system 600.

FIG. 6Q depicts a different example scenario in which Jill, the user of computer system 600, is able to complete her workout without interruption and/or without any check-in notifications being transmitted to remote user Ivy. For example, in some embodiments, FIG. 6Q depicts an example scenario in which, from FIG. 6L, the user of computer system 600 selected option 654a within the threshold amount of time, and/or an example scenario in which alert was never displayed during the user's workout. At FIG. 6Q, computer system 600 detects user input 671a, which is a swipe right input via touch-sensitive display 602. At FIG. 6R, in response to user input 671a, computer system 600 displays user interface 610. At FIG. 6R, computer system 600 detects user input 671b, which is a touch input (e.g., a tap input) corresponding to selection of option 610a to end the user's workout session.

At FIG. 6S, in response to user input 671b, computer system 600 ends the workout session and displays workout summary user interface 672. Workout summary user interface 672 includes one or more workout metrics pertaining to the workout session, including duration information 672a and distance information 672b. Additionally, in response to user input 671b and/or based on termination of the workout session, computer system 600 also terminates the workout-based check-in session, and displays notification 674 indicating that the workout-based check-in session has been ended, and remote user Ivy has been notified that the workout-based check-in session has ended. At FIG. 6S, in response to user input 671b, computer system 600 transmits information to remote user Ivy (e.g., to computer system 630 that is associated with remote user Ivy) indicating that the user of computer system 600 has ended their workout session, and that the workout-based check-in session is now terminated. At FIG. 6S, in response to receiving this information from computer system 600, computer system 630 displays notification 676, which notifies remote user Ivy that Jill has ended their workout session and that the workout-based check-in session is terminated. Notification 676 includes options 676a-676c, map 676d, and information 676e-676f. Option 676a, when selected, causes computer system 630 to cease display of notification 676. Option 676b, when selected, causes computer system 630 to initiate a telephone call and/or an audio call with Jill, the user of computer system 600. M ap 676d displays the location of computer system 600 and/or the user of computer system 600 when the workout session was ended and the workout-based check-in session was terminated. Information 676e indicates that Jill, the user of computer system 600, started their outdoor run workout at 9:41 PM. Information 676f indicates that Jill's workout session ended at 10:09 PM. At FIG. 6S, computer system 630 detects user input 677, which is a touch input (e.g., a tap input) corresponding to selection of option 676a.

At FIG. 6T, in response to user input 677, computer system 600 displays messaging session user interface 648. Messaging session user interface 648 now includes a new message, message 649b, that indicates that the workout-based check-in session has ended, and that Jill ended their workout session at 10:09 PM.

At FIG. 6U, computer system 600 displays workout selection user interface 606, which includes platter 606c. Platter 606c, when selected, causes computer system 600 to initiate a cycling workout. At FIG. 6U, computer system 600 detects user input 678a, which is a touch input corresponding to selection of platter 606c. At FIG. 6V, in response to user input 678a, computer system 600 initiates a cycling workout workout session and displays user interface 608. At FIG. 6V, computer system 600 detects user input 678b, which is a press (e.g., a depression) of rotatable and depressible input mechanism 604.

On the left side of FIG. 6W, in response to user input 678b, computer system 600 displays time user interface 680-1. Time user interface 680-1 includes time indication 682-1, and complications 683a-683c. Complication 683a corresponds to a fitness application and, when selected, causes computer system 600 to open the fitness application. Complication 683b corresponds to a health application, displays information from the health application (e.g., daily physical activity metric information) and, when selected, causes computer system 600 to open the health application. Complication 683c corresponds to a weather application, displays information from the health application (e.g., current and/or daily temperature information) and, when selected, causes computer system 600 to open the weather application. On the left side of FIG. 6W, based on a determination that a workout session is currently active, and that a workout-based check-in session is not active, computer system 600 displays suggestion 686-1 suggesting that the user initiate a workout-based check-in session. Additionally, in FIG. 6W, based on a determination that the user of computer system 600 has previously initiated a threshold number of workout-based check-in sessions with remote user Ivy, suggestion 686-1 suggests that the user initiate a workout-based check-in session with remote user Ivy. In some embodiments, suggestion 686-1, when selected, causes computer system 600 to display user interface 616 and/or enter the state shown in FIG. 6E.

The right side of FIG. 6W depicts an alternative embodiment in which, in response to user input 678b, computer system 600 displays a different time user interface, time user interface 680-2. Time user interface 680-2 includes current date indication 684 and current time indication 682-2. On the right side of FIG. 6W, based on a determination that a workout session is currently active, and that a workout-based check-in session is not active, computer system 600 displays suggestion 686-2 suggesting that the user initiate a workout-based check-in session. Additionally, in FIG. 6W, based on a determination that the user of computer system 600 has previously initiated a threshold number of workout-based check-in sessions with remote user Ivy, suggestion 686-2 suggests that the user initiate a workout-based check-in session with remote user Ivy. In some embodiments, suggestion 682-2, when selected, causes computer system 600 to display user interface 616 and/or enter the state shown in FIG. 6E.

FIG. 6X depicts a different scenario from FIG. 6W. On the left side of FIG. 6X, in response to user input 678b, computer system 600 displays time user interface 680-1. On the left side of FIG. 6X, based on a determination that a workout session is currently active, and that a workout-based check-in session is not active, computer system 600 displays suggestion 688-1 suggesting that the user initiate a workout-based check-in session. However, in FIG. 6X, suggestion 688-1 suggests that the user initiate workout-based check-in session without identifying any specific user for the workout-based check-in session. This may occur, for example, when the user of computer system 600 has not selected any particular contact for workout-based check-ins a threshold number of times, or suggestion 688-1, in some embodiments, is the default suggestion. In some embodiments, suggestion 688-1, when selected, causes computer system 600 to display user interface 612 and/or enter the state shown in FIG. 6D. Similarly, on the right side of FIG. 6X, computer system 600 displays time user interface 680-2 with suggestion 688-2. In some embodiments, suggestion 688-2, when selected, causes computer system 600 to display user interface 612 and/or enter the state shown in FIG. 6D.

In some embodiments, when a workout-based check-in session is active, computer system 600 displays time user interface 680-1 and/or time user interface 680-2 with an indication that a workout-based check-in session is active. For example, in FIG. 6Y, on the left side, computer system 600 displays time user interface 680-1 with indication 689-1 that is indicative of an active workout-based check-in session, and on the right side, computer system 600 displays time user interface 680-2 with indication 689-2 that is indicative of an active workout-based check-in session.

In some embodiments, the indications shown with workout session user interface 608, time user interface 680-1, and/or time user interface 680-2 change as the status of the workout-based check-in session changes. For example, in FIG. 6Z, computer system 600 displays user interface 608, time user interface 690-1, and time user interface 690-2 with indications 690, 690-1, and 690-2, respectively, indicating that computer system 600 does not have network connectivity. In some embodiments, computer system 600 connects to a communications network via another device (e.g., via a phone corresponds to the user of computer system 600), and indication 690-2 indicates that computer system 600 is not able to detect and/or connect with the other device and, as a result, does not have network connectivity. In FIG. 6AA, computer system 600 displays user interface 608, time user interface 690-1, and time user interface 690-2 with indications 691, 691-1, and 691-2, respectively, indicating that a check-in notification has been sent to a remote user, and that the location of the user of computer system 600 is being shared with the remote user.

FIG. 6A B depicts an example scenario and embodiment in which computer system 600 determines that the user has opted to initiate a workout-based check-in session a threshold number of times. Based on this determination, computer system 600 displays suggestion 692, which asks the user if the user would like to always automatically initiate a workout-based check-in session when the user starts a workout (e.g., without requiring additional user input to initiate the workout-based check-in session). At FIG. 6AB, computer system 600 detects user input 693, which is a touch input corresponding to selection of suggestion 692.

At FIG. 6AC, in response to user input 692, computer system 600 displays user interface 694. User interface 694 includes options 694a-694c. Option 694a, when selected, causes computer system 600 to cease display of user interface 694. Option 694b can be transitioned between an enabled state and a disabled state. When option 694b is in the enabled state, computer system 600 automatically initiates a workout-based check-in session whenever the user of computer system 600 initiates a workout session (e.g., by selecting a platter in user interface 606). When option 694b is in the disabled state, computer system 600 does not automatically initiate workout-based check-in sessions, and the user must provide user input to initiate workout-based check-in sessions (e.g., as shown in FIGS. 6A-6I above). Option 694c, when selected, causes computer system 600 to display a list of contacts (e.g., as in FIG. 6D) so that the user can select the contact for automatically-initiated workout-based check-in sessions. In FIG. 6AC, the user has selected remote user Ivy, and option 694b is enabled, such that whenever the user of computer system 600 starts a new workout session, computer system 600 will automatically (e.g., without additional user input) initiate a workout-based check-in session with remote user Ivy.

FIG. 7 is a flow diagram illustrating a method for managing workout-based notifications using a computer system in accordance with some embodiments. Method 700 is performed at a computer system (e.g., 100, 300, 500, and/or 600) (e.g., a smart phone, a smart watch, a tablet, a laptop, a desktop, a wearable device, wrist-worn device, and/or head-mounted device) that is in communication with one or more display generation components (e.g., 602) (e.g., a display, a touch-sensitive display, and/or a display controller) and one or more input devices (e.g., 602 and/or 604) (e.g., a touch-sensitive surface, a touch-sensitive display, a button, a rotatable input mechanism, a depressible and rotatable input mechanism, a camera, an accelerometer, an inertial measurement unit (IM U), a heartrate sensor, a body temperature sensor, and/or a blood-oxygen level sensor). Some operations in method 700 are, optionally, combined, the orders of some operations are, optionally, changed, and some operations are, optionally, omitted.

As described below, method 700 provides an intuitive way for managing workout-based notifications. The method reduces the cognitive burden on a user for managing workout-based notifications, thereby creating a more efficient human-machine interface. For battery-operated computing devices, enabling a user to manage workout-based notifications faster and more efficiently conserves power and increases the time between battery charges.

In some embodiments, the computer system (e.g., 600) detects (702), via the one or more input devices, a first user input (e.g., one or more user inputs) (e.g., one or more touch inputs, one or more hardware control inputs, one or more button presses, one or more rotational inputs, one or more gesture inputs, and/or one or more spoken inputs) corresponding to a user request to initiate a workout session (e.g., user input 607). In response to detecting the first user input, the computer system initiates (704) a workout session (e.g., FIG. 6B). While the workout session is active, the computer system detects (706), via the one or more input devices, one or more user inputs (e.g., one or more touch inputs, one or more hardware control inputs, one or more button presses, one or more rotational inputs, one or more gesture inputs, and/or one or more spoken inputs) (e.g., one or more user inputs that include selection of a check-in option) corresponding to a user request to activate a workout-based check-in session (e.g., a check-in session that is initiated and/or terminated based on initiation and/or termination of the workout session) with an external computer system separate from the computer system (e.g., in some embodiments, an external computer system that corresponds to an external user that is different from the user of the computer system) (e.g., user inputs 611, 615, 617, 621b, and/or 625), including selection of a displayed check-in option (e.g., 610e). In response to detecting the one or more user inputs corresponding to the user request to activate the workout-based check-in session with an external computer system (708), the computer system initiates (710) a process for activating a workout-based check-in session with a first external computer system (e.g., 630 and/or 640) (e.g., in some embodiments, an external computer system that corresponds to an external user that is different from the user of the computer system) separate from the computer system (e.g., 600), wherein, while the workout-based check-in session is active, the computer system automatically transmits notifications to the first external computer system based on satisfaction of notification criteria pertaining to the computer system (in some embodiments, while the workout-based check-in session is not active, the computer system does not automatically transmit notifications to the first external computer system based on satisfaction of the notification criteria). While the workout-based check-in session with the first external computer system is active (712): in accordance with a determination that notification criteria are satisfied, the computer system (e.g., 600) transmits (714) check-in notification information to the first external computer system (e.g., 630), wherein the check-in notification information causes the first external computer system to output information (e.g., 664, 668, 668-1, and/or 676) (e.g., display information and/or output audio information) corresponding to the computer system. In some embodiments, while the workout-based check-in session with the first external computer system is active: in accordance with a determination that notification criteria are not satisfied, the computer system forgoes transmitting check-in notification information to the first external computer system.

In some embodiments, initiating a workout session includes initiating recording (e.g., tracking, logging, collecting) of physical activity metrics corresponding to the workout session (e.g., physical activity metrics indicative of a physical activity level of the user during the workout session). In some embodiments, the physical activity metrics are recorded (e.g., captured) using one or more sensors (e.g., GPS, accelerometer, gyroscope, heart rate) of the computer system or an external device that is in communication with the computer system. In some embodiments, the physical activity metrics were not being recorded or were being recorded at a lower frequency and/or lower degree of precision prior to initiating (e.g., immediately prior to initiating) the workout session. In some embodiments, initiating the workout session includes causing one or more sensors to be enabled and/or activated to improve accurate measurements of user physical activity metrics during the workout session.

In some embodiments, when a check-in session is active and/or open, the computer system transmits (e.g., in some embodiments, automatically transmits) one or more notifications to the external computer system when one or more notification criteria are satisfied with respect to the computer system. In some embodiments, the notification criteria include a criterion that is satisfied when the computer system has displayed an affordance for a threshold duration of time (e.g., five minutes, ten minutes, fifteen minutes, or twenty minutes) without user input interacting with the displayed affordance (e.g., the user has not responded to a notification within a threshold duration of time). In some embodiments, the one or more notification criteria include a criterion that is satisfied when the computer system has not moved for a threshold duration of time (e.g., 15 minutes, 30 minutes, 45 minutes, or one hour). In some embodiments, the one or more notification criteria include a criterion that is satisfied when the computer system is moving at a rate of speed that exceeds a threshold rate of speed (e.g., greater than 20 mph, greater than 25 mph, greater than 30 mph, greater than 35 mph, greater than 40 mph, greater than 45 mph, and/or greater than 50 mph) (e.g., a rate of speed indicative of the user being in a motor vehicle). In some embodiments, the one or more notification criteria include a criterion that is satisfied when the computer system has lost connectivity and/or has limited connectivity with a network (e.g., a cellular network and/or an internet network). In some embodiments, the one or more notification criteria include a criterion that is satisfied when the computer system receives user input indicating a user request to send a notification to the external computer system. In some embodiments, the one or more notification criteria include a criterion that is satisfied when the computer system receives user input indicating a user request to output an emergency call (e.g., to first responders and/or to emergency responders). In some embodiments, the one or more notification criteria include a criterion that is satisfied when the computer system outputs an emergency call (e.g., to first responders and/or to emergency responders).

In some embodiments, when a check-in session is active and/or open, the computer system transmits one or more notifications to the external computer system when one or more notification criteria are satisfied with respect to the computer system. In some embodiments, the notification criteria include a criterion that is satisfied when the computer system has displayed a check-in notification affordance for a threshold duration of time (e.g., five minutes, ten minutes, fifteen minutes, or twenty minutes) without user input interacting with the displayed check-in notification affordance (e.g., the user has not responded to the check-in notification affordance within a threshold duration of time). In some embodiments, the computer system displays the check-in notification affordance when alert display criteria are satisfied. In some embodiments, the alert display criteria include a criterion that is satisfied when the computer system has not moved for a threshold duration of time (e.g., 15 minutes, 30 minutes, 45 minutes, or one hour). In some embodiments, the alert display criteria include a criterion that is satisfied when the computer system is moving at a rate of speed that exceeds a threshold rate of speed (e.g., greater than 20 mph, greater than 25 mph, greater than 30 mph, greater than 35 mph, greater than 40 mph, greater than 45 mph, and/or greater than 50 mph) (e.g., a rate of speed indicative of the user being in a motor vehicle). In some embodiments, the alert display criteria include a criterion that is satisfied when the computer system has lost connectivity and/or has limited connectivity with a network (e.g., a cellular network and/or an internet network. Providing the user with an option to initiate a workout-based check-in session allows the user to perform these operations with fewer inputs. Additionally, automatically transmitting a workout-based check-in notification when notification criteria are met and the workout-based check-in session is active allows these operations to be performed without user input. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, initiating the process for activating the workout-based check-in session with the first external computer system comprises: displaying, via the one or more display generation components (e.g., 602), a contact selection user interface (e.g., 612) that includes a plurality of contact options (e.g., 614a-614d) for the workout-based check-in session, including: a first contact option corresponding to a first contact (e.g., a first person, a first friend, a first family member, and/or a first phone number) that, when selected, causes the computer system to initiate a process for activating a workout-based check-in session with the first contact; and a second contact option corresponding to a second contact (e.g., a second person, a second friend, second first family member, and/or a second phone number) different from the first contact that, when selected, causes the computer system to initiate a process for activating a workout-based check-in session with the second contact. Displaying a list of contacts that a user can select from to initiate a workout-based check-in session enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, the first contact option (e.g., 614a) is displayed within a first region of the contact selection user interface (e.g., 612) (e.g., a top region of the contact selection user interface; and/or as a topmost contact option of the plurality of contact options) based on a determination that the user of the computer system has previously activated a workout-based check-in session with the first contact (e.g., in some embodiments, option 614a is displayed at the top of user interface 612 based on remote user Ivy having previously been selected for one or more workout-based check-in sessions). In some embodiments, contacts and/or people that have previously been selected for workout-based check-in sessions are prioritized (e.g., moved to the top of and/or otherwise visually emphasized) within the contact selection user interface. Displaying a list of contacts that a user can select from to initiate a workout-based check-in session, and automatically prioritizing previously-selected contacts, enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, while the workout session is active, the computer system concurrently displays, via the one or more display generation components: the check-in option (e.g., 610e) (e.g., a check-in option that, when selected, causes the computer system to initiate a process for activating a workout-based check-in session with an external computer system); and an end workout option (e.g., 610a) that, when selected, causes the computer system to initiate a process for ending the workout session (e.g., causes the computer system to end the workout session and/or to display a confirmation option that is selectable to end the workout session). In some embodiments, ending a workout session includes ending recording (e.g., tracking, logging, collecting) of physical activity metrics corresponding to the workout session (e.g., physical activity metrics indicative of a physical activity level of the user during the workout session). In some embodiments, the physical activity metrics are recorded (e.g., captured) using one or more sensors (e.g., GPS, accelerometer, gyroscope, heart rate) of the computer system or an external device that is in communication with the computer system. In some embodiments, ending a workout session includes recording one or more physical activity metrics at a lower frequency and/or lower degree of precision than during the workout session and/or while the workout session is active. In some embodiments, ending the workout session includes causing one or more sensors to be disabled and/or deactivated. Providing the user with an option to initiate a workout-based check-in session and providing the user with an option to end the workout session allows the user to perform these operations with fewer inputs. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, while the workout session is active, the computer system concurrently displays, via the one or more display generation components: the check-in option (e.g., 610e) (e.g., a check-in option that, when selected, causes the computer system to initiate a process for activating a workout-based check-in session with an external computer system); and a pause workout option (e.g., 610b) that, when selected, causes the computer system to pause the workout session (e.g., causes the computer system to pause a timer of the workout session and/or causes the computer system to pause measuring and/or recording one or more physical activity metrics of the workout session). Providing the user with an option to initiate a workout-based check-in session and providing the user with an option to pause the workout session allows the user to perform these operations with fewer inputs. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, while the workout-based check-in session with the first external computer system (e.g., 630 and/or 640) is active, the computer system detects that computer system movement criteria are satisfied (e.g., FIG. 6L), wherein the computer system movement criteria pertain to movement of the computer system (e.g., 600) (e.g., velocity of the computer system and/or acceleration of the computer system) (e.g., the computer system has stopped moving, or the computer system is moving at a rate of speed that is greater than a threshold rate of speed (e.g., 30 mph, 35 mph, 40 mph, or 45 mph)). In response to detecting that the computer system movement criteria are satisfied, the computer system displays a first alert (e.g., 654), wherein: the determination that notification criteria are satisfied includes a determination that the user has not interacted with (e.g., has not dismissed, has not responded to, and/or has not provided a particular type of user input with respect to) the first alert (e.g., 654) (e.g., the user has not selected option 654a) within a threshold duration of time (e.g., within 1 minute, within 2 minutes, within 5 minutes, within 10 minutes, or within 15 minutes since the first alert was displayed); and the check-notification information is transmitted to the first external computer system (e.g., 630 and/or 640) in accordance with a determination that the user has not interacted with the first alert within the threshold duration of time. Automatically transmitting a notification to the external computer system when notification criteria are satisfied allows for these operations to be performed without user input. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, while the workout-based check-in session with the first external computer system (e.g., 630 and/or 640) is active, the computer system detects one or more user inputs corresponding to a user request to end the workout-based check-in session (e.g., 656b) (e.g., one or more touch inputs, one or more hardware control inputs, one or more button presses, one or more rotational inputs, one or more gesture inputs, and/or one or more spoken inputs) (e.g., one or more user inputs corresponding to a user request to end the workout-based check-in session while maintaining and/or continuing the workout session). In response to detecting the one or more user inputs corresponding to the user request to end the workout-based check-in session, the computer system (e.g., 600) ends the workout-based check-in session, wherein: the determination that notification criteria are satisfied includes a determination that the user of the computer system has ended the workout-based check-in session; and the check-notification information is transmitted to the first external computer system in accordance with a determination that the user of the computer system has ended the workout-based check-in session (e.g., FIG. 6N). Automatically transmitting a notification to the external computer system when notification criteria are satisfied allows for these operations to be performed without user input. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, while the workout-based check-in session with the first external computer system (e.g., 630 and/or 640) is active, the computer system detects one or more user inputs corresponding to a user request to end the workout session (e.g., user input 671b) (e.g., one or more touch inputs, one or more hardware control inputs, one or more button presses, one or more rotational inputs, one or more gesture inputs, and/or one or more spoken inputs). In response to detecting the one or more user inputs corresponding to the user request to end the workout session, the computer system ends the workout session (e.g., FIG. 6S), wherein: the determination that notification criteria are satisfied includes a determination that the workout session has ended; and the check-notification information is transmitted to the first external computer system in accordance with a determination that workout session has ended (e.g., FIG. 6S). Automatically transmitting a notification to the external computer system when notification criteria are satisfied allows for these operations to be performed without user input. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, while the workout-based check-in session with the first external computer system is active, the computer system detects one or more user inputs corresponding to a user request to transmit an emergency call (e.g., an emergency call to one or more first responders and/or one or more emergency response services) (e.g., one or more touch inputs, one or more hardware control inputs, one or more button presses, one or more rotational inputs, one or more gesture inputs, and/or one or more spoken inputs). In response to detecting the one or more user inputs corresponding to the user request to transmit an emergency call, the computer system transmits an emergency call (e.g., to one or more first responders and/or one or more emergency response services), wherein: the determination that notification criteria are satisfied includes a determination that the user of the computer system has initiated an emergency call; and the check-notification information is transmitted to the first external computer system in accordance with a determination that the user of the computer system has initiated an emergency call. For example, in some embodiments, when computer system 600 transmits an emergency call (e.g., to first responders and/or to emergency response services), computer system 600 transmits information to computer system 630 indicating that computer system 600 has transmitted an emergency call. In some embodiments, in response to receiving this information, computer system 630 displays a notification similar to notification 668 indicating that computer system 600 made an emergency call (e.g., in some embodiments, the user interface displays the time the emergency call was made and/or the location of computer system 600 when the emergency call was made). Automatically transmitting a notification to the external computer system when notification criteria are satisfied allows for these operations to be performed without user input. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, while the workout-based check-in session with the first external computer system is active, the computer system detects that a connected device (e.g., a smart watch, a tablet, a phone, or other device) that was in communication with the computer system (e.g., a smart phone connected to computer system 600) is no longer in communication with the computer system (e.g., detecting that the user's smart watch is no longer connected to the user's phone or vice versa) (e.g., FIG. 6Z), wherein: the determination that notification criteria are satisfied includes a determination that the connected device is no longer in communication with the computer system; and the check-notification information is transmitted to the first external computer system in accordance with a determination that the connected device is no longer in communication with the computer system. For example, in some embodiments, when computer system 600 loses network connectivity and/or disconnects from a smart phone that was providing computer system 600 with network connectivity, computer system 600 and/or the smart phone transmits information to computer system 630 indicating that computer system 600 has been disconnected from a communications network. In some embodiments, in response to receiving this information, computer system 630 displays a notification similar to notification 668 indicating that computer system 600 has disconnected from a communications network and/or has lost network connectivity (e.g., in some embodiments, the user interface displays the time computer system 600 lost network connectivity and/or the location of computer system 600 when computer system 600 lost network connectivity). Automatically transmitting a notification to the external computer system when notification criteria are satisfied allows for these operations to be performed without user input. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, while the workout session is active, the computer system displays, via the one or more display generation components, a workout session user interface (e.g., 608), wherein the workout session user interface displays one or more physical activity metrics that correspond to (e.g., are recorded during) the workout session (e.g., workout duration, heart rate, calories burned, distance traveled, and/or repetitions completed), and displaying the workout session user interface includes: in accordance with a determination that a workout-based check-in session is active, displaying (e.g., within the workout session user interface) a check-in indication (e.g., 626) that is indicative of an active workout-based check-in session (e.g., FIG. 6I); and in accordance with a determination that workout-based check-in session is not active, forgoing display of the check-in indication (e.g., FIG. 6B). Displaying an indication when a workout-based check-in session is active provides the user with visual feedback about a state of the system (e.g., that a workout-based check-in session is active), thereby improving visual feedback to the user. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, while the workout-based check-in session with the first external computer system is active, the computer system detects that user notification criteria are satisfied; and in response to detecting that the user notification criteria are satisfied, the computer system displays, via the one or more display generation components, a first user interface (e.g., 654) that pertains to the workout-based check-in session. In some embodiments, the user notification criteria includes computer system movement criteria, wherein the computer system movement criteria pertain to movement of the computer system (e.g., velocity of the computer system and/or acceleration of the computer system) (e.g., the computer system has stopped moving, or the computer system is moving at a rate of speed that is greater than a threshold rate of speed (e.g., 30 mph, 35 mph, 40 mph, or 45 mph)). In some embodiments, the user notification criteria include criteria that pertain to a determination that the user has been disrupted in performing their workout and/or the user no longer performing the workout. Automatically displaying the first user interface when the user notification criteria are satisfied allows for these operations to be performed without user input. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, the first user interface (e.g., 654) includes a continue check-in option (e.g., 654a and/or 654c) that, when selected, causes the computer system to maintain the workout-based check-in session without transmitting check-in notification information to the first external computer system. In some embodiments, while displaying the first user interface, the computer system detects, via the one or more input devices, a selection input (e.g., one or more user inputs) (e.g., one or more touch inputs, one or more hardware control inputs, one or more button presses, one or more rotational inputs, one or more gesture inputs, and/or one or more spoken inputs) corresponding to selection of the continue check-in option; and in response to detecting the selection input corresponding to selection of the continue check-in option, the computer system maintains the workout-based check-in session without transmitting check-in notification information to the first external computer system. Automatically displaying the first user interface when the user notification criteria are satisfied allows for these operations to be performed without user input. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, the first user interface (e.g., 654) includes an end check-in option (e.g., 654b) that, when selected, causes the computer system to end the workout-based check-in session (or, in some embodiments, initiate a process for ending the workout-based check-in session). In some embodiments, while displaying the first user interface, the computer system detects, via the one or more input devices, a selection input (e.g., user input 656b) (e.g., one or more user inputs) (e.g., one or more touch inputs, one or more hardware control inputs, one or more button presses, one or more rotational inputs, one or more gesture inputs, and/or one or more spoken inputs) corresponding to selection of the end check-in option (e.g., 654b). In response to detecting the selection input corresponding to selection of the end check-in option, the computer system causes the workout-based check-in session to be terminated; and transmits, to the first external computer system (e.g., 630), check-in termination information which causes the first external computer system to display an indication that the workout-based check-in session has been terminated (e.g., 664 in FIG. 6N). In some embodiments, when the workout-based check-in session is terminated, the computer system ceases automatic transmission of notifications pertaining to the computer system and/or the workout based on satisfaction of the notification criteria. Automatically displaying the first user interface when the user notification criteria are satisfied allows for these operations to be performed without user input. Additionally, displaying an option to end the workout-based check-in session allows for these operations to be performed with fewer user inputs. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, while the workout-based check-in session with the first external computer system is active: in accordance with a determination that the first user interface (e.g., 654) has been displayed for a threshold duration of time (e.g., one minute, two minutes, five minutes, ten minutes, or fifteen minutes) without user input interacting with the first user interface (e.g., without the user selecting option 654a) (e.g., the user of the computer system has not provided any input interacting with the first user interface and/or has not provided a particular type of input interacting with the first user interface), the computer system transmits, to the first external computer system (e.g., 630), first check-in notification information pertaining to the workout-based check-in session that causes the first external computer system to output first information (e.g., 668) (e.g., display information and/or output audio information) corresponding to the computer system, wherein the first check-in notification information includes information pertaining to a location (e.g., a geographic location) of the computer system and the first check-in notification information causes the first external computer system to display information pertaining to the location of the computer system (e.g., 668d). In some embodiments, while the workout-based check-in session with the first external computer system is active: in accordance with a determination that the first user interface has not been displayed for the threshold duration of time (e.g., one minute, two minutes, five minutes, ten minutes, or fifteen minutes) without user input interacting with the first user interface, the computer system forgoes transmitting the first check-in notification information to the first external computer system. Automatically transmitting check-in information to the first external computer system when the user does not interact with the first user interface for a threshold duration of time allows for these operations to be performed without user input. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, while the workout-based check-in session with the first external computer system is active: in accordance with a determination that the first user interface (e.g., 654) has been displayed for the threshold duration of time without user input interacting with the first user interface (e.g., without user input selecting option 654a), the computer system ceases display of the first user interface (e.g., 654); and displays, via the one or more display generation components, a second user interface (e.g., 666) different from the first user interface, wherein the second user interface indicates that first check-in notification information has been transmitted to the first external computer system. In some embodiments, the second user interface indicates that the location of the computer system is being shared with the first external computer system. In some embodiments, while the workout-based check-in session with the first external computer system is active: in accordance with a determination that the first user interface has not been displayed for the threshold duration of time (e.g., one minute, two minutes, five minutes, ten minutes, or fifteen minutes) without user input interacting with the first user interface, the computer system forgoes displaying the second user interface and/or maintains display of the first user interface without displaying the second user interface. Automatically displaying the second user interface when the user does not interact with the first user interface for a threshold duration of time provides the user with visual feedback about a state of the system (e.g., the computer system has transmitted notification information to the first external computer system). Doing so also allows for these operations to be performed without user input. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, the second user interface (e.g., 666) includes a stop sharing option (e.g., 666c) that, when selected, causes the computer system to cease sharing location information pertaining to the location of the computer system with the first external computer system (e.g., 630). In some embodiments, while displaying the second user interface, the computer system detects, via the one or more input devices, a selection input (e.g., one or more user inputs) (e.g., one or more touch inputs, one or more hardware control inputs, one or more button presses, one or more rotational inputs, one or more gesture inputs, and/or one or more spoken inputs) corresponding to selection of the stop sharing option; and in response to detecting the selection input corresponding to selection of the stop sharing option, the computer system ceases transmitting location information pertaining to the location of the computer system to the first external computer system. Automatically displaying the second user interface when the user does not interact with the first user interface for a threshold duration of time provides the user with visual feedback about a state of the system (e.g., the computer system has transmitted notification information to the first external computer system). Doing so also allows for these operations to be performed without user input. Additionally, displaying an option to stop sharing location information with the first external computer system allows for these operations to be performed with fewer user inputs, and also improves device security by allowing the user of the computer system to control if and/or when location information is shared with an external device. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, while the workout-based check-in session is active, and while displaying the workout session user interface (e.g., 608), the computer system detects that the computer system has transitioned from a first power mode to a low power mode (e.g., in response to one or more user inputs and/or automatically (e.g., based on the computer system falling below a power level and/or battery level threshold) d), wherein, in the low power mode, the computer system activates one or more power saving measures that are not activated in the first power mode. In response to detecting that the computer system has transitioned from the first power mode to the low power mode, the computer system (e.g., 600) updates the check-in indication (e.g., 626) to indicate that the computer system is in the low power mode. For example, in some embodiments, when computer system 600 enters a low power mode, indication 626 is updated to indicate that computer system 600 is in the lower power mode (e.g., similar to how in FIG. 6Z, indication 626 is updated to indication 690 to indicate that computer system 600 has lost network connectivity). In some embodiments, in the low power mode, the computer system (e.g., 600) decreases a brightness of the one or more display generation components (e.g., 602) compared to when the computer system is in the first power mode. In some embodiments, in the low power mode, the computer system (e.g., 600) decreases a frequency with which the one or more display generation components (e.g., 602) are updated compared to when the computer system is in the first power mode. In some embodiments, in the low power mode, the computer system decreases the frequency with which the computer system pulls and/or receives data from external computer systems compared to when the computer system is in the first power mode. In some embodiments, in the low power mode, the computer system turns off a cellular connection of the computer system until the user provides specific input requesting and/or requiring cellular data transmission (e.g., user input to transmit a message; and/or user input requesting loading of data and/or content that requires receiving data); whereas in the first power mode, the computer system periodically and/or automatically transmits and/or receives cellular data even without user input requesting and/or requiring cellular data transmission. Automatically updating the check-in indication when the computer system enters the low power mode provides the user with visual feedback about a state of the system (e.g., the computer system is in the low power mode). Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, in response to detecting that the computer system (e.g., 600) has transitioned from the first power mode to the low power mode, the computer system transmits low power information to the first external computer system (e.g., 630) that causes the first external computer system to display an indication that the computer system is in the low power mode (e.g., in some embodiments, when computer system 600 enters a low power mode, computer system 600 transmits information to remote computer system 630 that causes remote computer system 630 to display a notification (e.g., similar to notifications 668 and/or 676) indicating that computer system 600 has entered a low power mode). In some embodiments, when the computer system transitions from the first power mode to the low power mode, and the workout-based check-in session is not active, the computer system does not transmit information to the first external computer system that causes the first external computer system to display an indication that the computer system is in the low power mode. Automatically transmitting information to the first external computer system when the computer system enters the low power mode allows for these operations to be performed without user input. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, the computer system displays, via the one or more display generation components, a messaging session user interface (e.g., 648 and/or 650) that corresponds to a messaging session that includes the user of the computer system and a first remote user that is associated with the first external computer system. While displaying the messaging session user interface, the computer system detects, via the one or more display generation components, a first set of user inputs (e.g., one or more user inputs) (e.g., one or more touch inputs, one or more hardware control inputs, one or more button presses, one or more rotational inputs, one or more gesture inputs, and/or one or more spoken inputs). In response to detecting the first set of user inputs: in accordance with a determination that a workout session is active when the first set of user inputs is detected, the computer system displays, via the one or more display generation components, a selectable user interface object that, when selected, causes the computer system to initiate a process for activating a workout-based check-in session with a respective external computer system separate from the computer system; and in accordance with a determination that a workout session is not active when the first set of user inputs is detected, the computer system (e.g., 600) forgoes display of the selectable user interface object (e.g., in some embodiments, displaying a non-selectable representation of the selectable user interface object, or displaying a different user interface object that does not initiate a workout-based check-in session). In some embodiments, computer system 600 displays a messaging session user interface similar to messaging session user interface 648 (e.g., in some embodiments, computer system 600 displays messaging session user interface 648 rather than computer system 630). In some embodiments, while computer system 600 is displaying messaging session user interface 648, computer system 600 detects user input corresponding to selection of option 652b. In some embodiments, if a workout session is active when the user selects option 652b, computer system 600 displays an option to initiate a workout-based check-in session (e.g., an option that, when selected, causes computer system 600 to enter the state shown in FIG. 6E). In some embodiments, if a workout session is not active when the user selects option 652b, computer system 600 does not display an option to initiate a workout-based check-in session and/or displays the option in an unselectable and/or disabled state. In some embodiments, while computer system 630 is displaying messaging session user interface 648, computer system 630 detects user input corresponding to selection of option 652b. In some embodiments, if a workout session is active on computer system 630 when the user selects option 652b, computer system 630 displays an option to initiate a workout-based check-in session (e.g., an option that, when selected, causes computer system 630 to enter the state for computer system 600 shown in FIG. 6E). In some embodiments, if a workout session is not active on computer system 630 when the user selects option 652b, computer system 630 does not display an option to initiate a workout-based check-in session and/or displays the option in an unselectable and/or disabled state. In some embodiments, displaying the messaging session user interface includes displaying a selectable user interface object that is associated with activating a workout-based check-in session with a respective external computer system separate from the computer system. While displaying the selectable user interface object, the computer system detects a selection input corresponding to selection of the selectable user interface object. In response to detecting the selection input corresponding to selection of the selectable user interface object: in accordance with a determination that a workout session is active when the selection input is detected, the computer system initiates a process for activating a workout-based check-in session with a respective external computer system separate from the computer system; and in accordance with a determination that a workout session is not active when the selection input is detected, the computer system forgoes initiating the process for activating a workout-based check-in session with a respective external computer system and, in some embodiments, displays an indication that a workout session must be active in order to activate a workout-based check-in session. Displaying an option to initiate a workout-based check-in session only when a workout is active enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

Note that details of the processes described above with respect to method 700 (e.g., FIG. 7) are also applicable in an analogous manner to the methods described below. For example, method 800 optionally includes one or more of the characteristics of the various methods described above with reference to method 700. For example, in some embodiments, the workout session recited in method 700 is the same workout session recited in method 800; and/or the workout-based check-in session recited in method 700 is the same workout-based check-in session recited in method 800. For brevity, these details are not repeated below.

FIG. 8 is a flow diagram illustrating a method for managing workout-based notifications using a computer system in accordance with some embodiments. Method 800 is performed at a computer system (e.g., 100, 300, 500, and/or 600) (e.g., a smart phone, a smart watch, a tablet, a laptop, a desktop, a wearable device, wrist-worn device, and/or head-mounted device) that is in communication with one or more display generation components (e.g., 602) (e.g., a display, a touch-sensitive display, and/or a display controller) and one or more input devices (e.g., 602 and/or 604) (e.g., a touch-sensitive surface, a touch-sensitive display, a button, a rotatable input mechanism, a depressible and rotatable input mechanism, a camera, an accelerometer, an inertial measurement unit (IM U), a heartrate sensor, a body temperature sensor, and/or a blood-oxygen level sensor). Some operations in method 800 are, optionally, combined, the orders of some operations are, optionally, changed, and some operations are, optionally, omitted.

As described below, method 800 provides an intuitive way for managing workout-based notifications. The method reduces the cognitive burden on a user for managing workout-based notifications, thereby creating a more efficient human-machine interface. For battery-operated computing devices, enabling a user to manage workout-based notifications faster and more efficiently conserves power and increases the time between battery charges.

In some embodiments, the computer system displays (802), via the one or more display generation components, a time user interface (e.g., 680-1 and/or 680-2) (e.g., a user interface that includes an analog and/or digital indication of time, a clock face user interface, a watch face user interface, a reduced-power screen, a wake screen, and/or a lock screen), wherein the time user interface includes an indication of time (e.g., 682-1 and/or 682-2) (e.g., an analog and/or digital indication of time). While displaying the time user interface (804): in accordance with a determination that a workout session is active (e.g., at the time of displaying the time user interface) (806), the computer system displays (808), via the one or more display generation components (e.g., concurrently with the time user interface), a recommendation (e.g., 686-1, 686-2, 688-1, and/or 688-2) to initiate a workout-based check-in session with an external computer system, wherein, while the workout-based check-in session is active, the computer system (e.g., 600) automatically transmits notifications to the external computer system based on satisfaction of notification criteria pertaining to the computer system.

In some embodiments, initiating a workout session includes initiating recording (e.g., tracking, logging, collecting) of physical activity metrics corresponding to the workout session (e.g., physical activity metrics indicative of a physical activity level of the user during the workout session). In some embodiments, the physical activity metrics are recorded (e.g., captured) using one or more sensors (e.g., GPS, accelerometer, gyroscope, heart rate) of the computer system or an external device that is in communication with the computer system. In some embodiments, the physical activity metrics were not being recorded or were being recorded at a lower frequency and/or lower degree of precision prior to initiating (e.g., immediately prior to initiating) the workout session. In some embodiments, initiating the workout session includes causing one or more sensors to be enabled and/or activated to improve accurate measurements of user physical activity metrics during the workout session.

In some embodiments, when a check-in session is active and/or open, the computer system transmits (e.g., in some embodiments, automatically transmits) one or more notifications to the external computer system when one or more notification criteria are satisfied with respect to the computer system. In some embodiments, the notification criteria include a criterion that is satisfied when the computer system has displayed an affordance for a threshold duration of time (e.g., five minutes, ten minutes, fifteen minutes, or twenty minutes) without user input interacting with the displayed affordance (e.g., the user has not responded to a notification within a threshold duration of time). In some embodiments, the one or more notification criteria include a criterion that is satisfied when the computer system has not moved for a threshold duration of time (e.g., 15 minutes, 30 minutes, 45 minutes, or one hour). In some embodiments, the one or more notification criteria include a criterion that is satisfied when the computer system is moving at a rate of speed that exceeds a threshold rate of speed (e.g., greater than 20 mph, greater than 25 mph, greater than 30 mph, greater than 35 mph, greater than 40 mph, greater than 45 mph, and/or greater than 50 mph) (e.g., a rate of speed indicative of the user being in a motor vehicle). In some embodiments, the one or more notification criteria include a criterion that is satisfied when the computer system has lost connectivity and/or has limited connectivity with a network (e.g., a cellular network and/or an internet network). In some embodiments, the one or more notification criteria include a criterion that is satisfied when the computer system receives user input indicating a user request to send a notification to the external computer system. In some embodiments, the one or more notification criteria include a criterion that is satisfied when the computer system receives user input indicating a user request to output an emergency call (e.g., to first responders and/or to emergency responders). In some embodiments, the one or more notification criteria include a criterion that is satisfied when the computer system outputs an emergency call (e.g., to first responders and/or to emergency responders).

In some embodiments, when a check-in session is active and/or open, the computer system transmits one or more notifications to the external computer system when one or more notification criteria are satisfied with respect to the computer system. In some embodiments, the notification criteria include a criterion that is satisfied when the computer system has displayed a check-in notification affordance for a threshold duration of time (e.g., five minutes, ten minutes, fifteen minutes, or twenty minutes) without user input interacting with the displayed check-in notification affordance (e.g., the user has not responded to the check-in notification affordance within a threshold duration of time). In some embodiments, the computer system displays the check-in notification affordance when alert display criteria are satisfied. In some embodiments, the alert display criteria include a criterion that is satisfied when the computer system has not moved for a threshold duration of time (e.g., 15 minutes, 30 minutes, 45 minutes, or one hour). In some embodiments, the alert display criteria include a criterion that is satisfied when the computer system is moving at a rate of speed that exceeds a threshold rate of speed (e.g., greater than 20 mph, greater than 25 mph, greater than 30 mph, greater than 35 mph, greater than 40 mph, greater than 45 mph, and/or greater than 50 mph) (e.g., a rate of speed indicative of the user being in a motor vehicle). In some embodiments, the alert display criteria include a criterion that is satisfied when the computer system has lost connectivity and/or has limited connectivity with a network (e.g., a cellular network and/or an internet network. Automatically displaying the recommendation to initiate a workout-based check-in session when a workout is active provides the user with feedback about a state of the system (e.g., that a workout is active and the computer system is able to start a workout-based check-in session). Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, while displaying the time user interface (e.g., 680-1 and/or 680-2): in accordance with a determination that a workout session is not active (e.g., at the time of displaying the time user interface), the computer system displays, via the one or more display generation components (e.g., concurrently with the time user interface), the time user interface without displaying a recommendation to initiate a workout-based check-in session with an external computer system (e.g., displays time user interface 680-1 and/or time user interface 680-2 without suggestion 686-1, 686-2, 688-1, and/or 688-2). Forgoing display of a recommendation to initiate a workout-based check-in session when a workout is not active enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, the recommendation (e.g., 686-1 and/or 686-2) identifies (e.g., recommends) a first contact (e.g., a first person, a first phone number, and/or a first name) for the workout-based check-in session. In some embodiments, the first contact is selected for inclusion in the recommendation based on the user of the computer system having previously selected the first contact for one or more workout-based check-in session. In some embodiments, the recommendation identifies and/or includes a particular contact (e.g., a first contact) based on and/or in accordance with a determination that the user of the computer system has previously selected the particular contact for workout-based check-in sessions a threshold number of times. In some embodiments, the recommendation, when selected, causes the computer system to initiate a process for activating a workout-based check-in session with the first contact and/or a first external computer system that is associated with the first contact. Automatically displaying the recommendation to initiate a workout-based check-in session when a workout is active provides the user with feedback about a state of the system (e.g., that a workout is active and the computer system is able to start a workout-based check-in session). Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, while displaying the recommendation (e.g., 688-1 and/or 688-2), the computer system detects, via the one or more input devices, a selection input (e.g., one or more user inputs) (e.g., one or more touch inputs, one or more hardware control inputs, one or more button presses, one or more rotational inputs, one or more gesture inputs, and/or one or more spoken inputs) corresponding to selection of the recommendation. In response to detecting the selection input corresponding to selection of the recommendation, the computer system displays, via the one or more display generation components, a contact selection user interface (e.g., 612) that includes a plurality of contact options (e.g., 614a-614d) for the workout-based check-in session, including: a first contact option corresponding to a first contact (e.g., a first person, a first friend, a first family member, and/or a first phone number) that, when selected, causes the computer system to initiate a process for activating a workout-based check-in session with the first contact; and a second contact option corresponding to a second contact (e.g., a second person, a second friend, second first family member, and/or a second phone number) different from the first contact that, when selected, causes the computer system to initiate a process for activating a workout-based check-in session with the second contact. In some embodiments, the recommendation does not identify and/or recommend any particular contact for the workout-based check-in session. In some embodiments, based on and/or in accordance with a determination that the user of the computer system has not previously selected a particular contact (e.g., the same contact) a threshold number of times for previous workout-based check in sessions, the computer system does not identify and/or recommend any particular contact within the recommendation and/or displays the recommendation without identifying and/or recommendation any particular contact within the recommendation. Displaying a list of contacts that a user can select from to initiate a workout-based check-in session enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, while displaying the recommendation (e.g., 686-1, 686-2, 688-1, and/or 688-2), the computer system detects, via the one or more input devices, a first set of user inputs (e.g., one or more user inputs) (e.g., one or more touch inputs, one or more hardware control inputs, one or more button presses, one or more rotational inputs, one or more gesture inputs, and/or one or more spoken inputs) that include selection of the recommendation. In response to detecting the first set of user inputs, the computer system initiates a workout-based check-in session with a first external computer system different from the computer system. For example, in some embodiments, selection of recommendation 686-1 or 686-2 causes computer system 600 to enter the state shown in FIG. 6E, and/or selection of recommendation 688-1 or 688-2 causes computer system 600 to enter the state shown in FIG. 6D, and the user can proceed from FIG. 6D or FIG. 6E to initiate a workout-based check-in session. While the workout-based check-in session with the first external computer system is active: in accordance with a determination that notification criteria are satisfied, the computer system (e.g., 600) transmits check-in notification information to the first external computer system (e.g., 630), wherein the check-in notification information causes the first external computer system to output information (e.g., display information and/or output audio information) corresponding to the computer system (e.g., 664, 668, and/or 676). In some embodiments, while the workout-based check-in session with the first external computer system is active: in accordance with a determination that notification criteria are not satisfied, the computer system forgoes transmitting check-in notification information to the first external computer system (e.g., forgoes causing the first external computer system to output information pertaining to the computer system and/or the workout-based check-in session). Automatically transmitting a workout-based check-in notification when notification criteria are met and the workout-based check-in session is active allows these operations to be performed without user input. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, while the workout-based check-in session with the first external computer system (e.g., 630) is active, the computer system (e.g., 600) detects that computer system movement criteria are satisfied, wherein the computer system movement criteria pertain to movement of the computer system (e.g., velocity of the computer system and/or acceleration of the computer system) (e.g., the computer system has stopped moving, or the computer system is moving at a rate of speed that is greater than a threshold rate of speed (e.g., 30 mph, 35 mph, 40 mph, or 45 mph)) (e.g., FIG. 6L). In response to detecting that the computer system movement criteria are satisfied, the computer system displays a first alert (e.g., 654), wherein: the determination that notification criteria are satisfied includes a determination that the user has not interacted with (e.g., has not dismissed, has not responded to, and/or has not provided a particular type of user input with respect to) the first alert within a threshold duration of time (e.g., within 1 minute, within 2 minutes, within 5 minutes, within 10 minutes, or within 15 minutes since and/or while the first alert was displayed) (e.g., the user has not selected option 654a within the threshold duration of time); and the check-notification information is transmitted to the first external computer system in accordance with a determination that the user has not interacted with the first alert within the threshold duration of time (e.g., the user has not selected option 654a within the threshold duration of time). Automatically transmitting a notification to the external computer system when notification criteria are satisfied allows for these operations to be performed without user input. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, while the workout-based check-in session with the first external computer system (e.g., 630) is active, the computer system detects one or more user inputs corresponding to a user request to end the workout-based check-in session (e.g., user input 656b) (e.g., one or more touch inputs, one or more hardware control inputs, one or more button presses, one or more rotational inputs, one or more gesture inputs, and/or one or more spoken inputs) (e.g., one or more user inputs corresponding to a user request to end the workout-based check-in session while maintaining and/or continuing the workout session). In response to detecting the one or more user inputs corresponding to the user request to end the workout-based check-in session, the computer system ends the workout-based check-in session (e.g., FIG. 6N), wherein: the determination that notification criteria are satisfied includes a determination that the user of the computer system has ended the workout-based check-in session; and the check-notification information is transmitted to the first external computer system in accordance with a determination that the user of the computer system has ended the workout-based check-in session (e.g., FIG. 6N). Automatically transmitting a notification to the external computer system when notification criteria are satisfied allows for these operations to be performed without user input. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, while the workout-based check-in session with the first external computer system (e.g., 630) is active, the computer system (e.g., 600) detects one or more user inputs corresponding to a user request to end the workout session (e.g., 671b) (e.g., one or more touch inputs, one or more hardware control inputs, one or more button presses, one or more rotational inputs, one or more gesture inputs, and/or one or more spoken inputs). In response to detecting the one or more user inputs corresponding to the user request to end the workout session, the computer system ends the workout session (e.g., FIG. 6S), wherein: the determination that notification criteria are satisfied includes a determination that the workout session has ended; and the check-notification information is transmitted to the first external computer system in accordance with a determination that workout session has ended (e.g., FIG. 6S). Automatically transmitting a notification to the external computer system when notification criteria are satisfied allows for these operations to be performed without user input. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, while the workout-based check-in session with the first external computer system (e.g., 630) is active, the computer system (e.g., 600) detects one or more user inputs corresponding to a user request to transmit an emergency call (e.g., an emergency call to one or more first responders and/or one or more emergency response services) (e.g., one or more touch inputs, one or more hardware control inputs, one or more button presses, one or more rotational inputs, one or more gesture inputs, and/or one or more spoken inputs). In response to detecting the one or more user inputs corresponding to the user request to transmit an emergency call, the computer system transmits an emergency call (e.g., to one or more first responders and/or one or more emergency response services), wherein: the determination that notification criteria are satisfied includes a determination that the user of the computer system has initiated an emergency call; and the check-notification information is transmitted to the first external computer system in accordance with a determination that the user of the computer system has initiated an emergency call. For example, in some embodiments, when computer system 600 transmits an emergency call (e.g., to first responders and/or to emergency response services), computer system 600 transmits information to computer system 630 indicating that computer system 600 has transmitted an emergency call. In some embodiments, in response to receiving this information, computer system 630 displays a notification similar to notification 668 indicating that computer system 600 made an emergency call (e.g., in some embodiments, the user interface displays the time the emergency call was made and/or the location of computer system 600 when the emergency call was made). Automatically transmitting a notification to the external computer system when notification criteria are satisfied allows for these operations to be performed without user input. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, while the workout-based check-in session with the first external computer system is active, the computer system detects that a connected device (e.g., a smart watch, a tablet, a phone, or other device) that was in communication with the computer system (e.g., 600) is no longer in communication with the computer system (e.g., detecting that the user's smart watch is no longer connected to the user's phone or vice versa) (e.g., FIG. 6Z), wherein: the determination that notification criteria are satisfied includes a determination that the connected device is no longer in communication with the computer system; and the check-notification information is transmitted to the first external computer system in accordance with a determination that the connected device is no longer in communication with the computer system. For example, in some embodiments, when computer system 600 loses network connectivity and/or disconnects from a smart phone that was providing computer system 600 with network connectivity, computer system 600 and/or the smart phone transmits information to computer system 630 indicating that computer system 600 has been disconnected from a communications network. In some embodiments, in response to receiving this information, computer system 630 displays a notification similar to notification 668 indicating that computer system 600 has disconnected from a communications network and/or has lost network connectivity (e.g., in some embodiments, the user interface displays the time computer system 600 lost network connectivity and/or the location of computer system 600 when computer system 600 lost network connectivity). Automatically transmitting a notification to the external computer system when notification criteria are satisfied allows for these operations to be performed without user input. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, displaying the time user interface (e.g., 680-1 and/or 680-2) further includes: in accordance with a determination that a workout-based check-in session is active, displaying (e.g., within the time user interface) a check-in indication (e.g., 689-1 and/or 689-2) that is indicative of an active workout-based check-in session; and in accordance with a determination that workout-based check-in session is not active, forgoing display of the check-in indication. Displaying an indication when a workout-based check-in session is active provides the user with visual feedback about a state of the system (e.g., that a workout-based check-in session is active), thereby improving visual feedback to the user. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, while the workout-based check-in session is active, and while displaying the time user interface (e.g., 680-1 and/or 680-2) including the check-in indication (e.g., 689-1 and/or 689-2), the computer system detects that the computer system has transitioned from a first power mode to a low power mode (e.g., in response to one or more user inputs and/or automatically (e.g., based on the computer system falling below a power level and/or battery level threshold) d), wherein, in the low power mode, the computer system activates one or more power saving measures that are not activated in the first power mode. In response to detecting that the computer system has transitioned from the first power mode to the low power mode, the computer system updates the check-in indication to indicate that the computer system is in the low power mode. For example, in some embodiments, when computer system 600 enters a low power mode, indication 689-1 and/or 689-2 is updated to indicate that computer system 600 is in the lower power mode (e.g., similar to how in FIG. 6Z, indication 689-1 is updated to indication 690-1 and/or indication 689-2 is updated to indication 690-2 to indicate that computer system 600 has lost network connectivity). In some embodiments, in the low power mode, the computer system decreases a brightness of the one or more display generation components compared to when the computer system is in the first power mode. In some embodiments, in the low power mode, the computer system decreases a frequency with which the one or more display generation components are updated compared to when the computer system is in the first power mode. In some embodiments, in the low power mode, the computer system decreases the frequency with which the computer system pulls and/or receives data from external computer systems compared to when the computer system is in the first power mode. In some embodiments, in the low power mode, the computer system turns off a cellular connection of the computer system until the user provides specific input requesting and/or requiring cellular data transmission (e.g., user input to transmit a message; and/or user input requesting loading of data and/or content that requires receiving data); whereas in the first power mode, the computer system periodically and/or automatically transmits and/or receives cellular data even without user input requesting and/or requiring cellular data transmission. Automatically updating the check-in indication when the computer system enters the low power mode provides the user with visual feedback about a state of the system (e.g., the computer system is in the low power mode). Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, in response to detecting that the computer system (e.g., 600) has transitioned from the first power mode to the low power mode, the computer system transmits low power information to the first external computer system (e.g., 630) that causes the first external computer system to display an indication that the computer system is in the low power mode (e.g., in some embodiments, when computer system 600 enters a low power mode, computer system 600 transmits information to remote computer system 630 that causes remote computer system 630 to display a notification (e.g., similar to notifications 668 and/or 676) indicating that computer system 600 has entered a low power mode). In some embodiments, when the computer system transitions from the first power mode to the low power mode, and the workout-based check-in session is not active, the computer system does not transmit information to the first external computer system that causes the first external computer system to display an indication that the computer system is in the low power mode. Automatically transmitting information to the first external computer system when the computer system enters the low power mode allows for these operations to be performed without user input. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, while a workout-based check-in session with a first external computer system (e.g., 630) is active, the computer system (e.g., 600) detects that user notification criteria are satisfied (e.g., FIG. 6L). In response to detecting that the user notification criteria are satisfied, the computer system displays, via the one or more display generation components, a first user interface (e.g., 654) that pertains to the workout-based check-in session. In some embodiments, the user notification criteria includes computer system movement criteria, wherein the computer system movement criteria pertain to movement of the computer system (e.g., velocity of the computer system and/or acceleration of the computer system) (e.g., the computer system has stopped moving, or the computer system is moving at a rate of speed that is greater than a threshold rate of speed (e.g., 30 mph, 35 mph, 40 mph, or 45 mph)). In some embodiments, the user notification criteria include criteria that pertain to a determination that the user has been disrupted in performing their workout and/or the user no longer performing the workout. Automatically displaying the first user interface when the user notification criteria are satisfied allows for these operations to be performed without user input. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, the first user interface (e.g., 654) includes a continue check-in option (e.g., 654a and/or 654c) that, when selected, causes the computer system to maintain the workout-based check-in session without transmitting check-in notification information to the first external computer system. In some embodiments, while displaying the first user interface, the computer system detects, via the one or more input devices, a selection input (e.g., one or more user inputs) (e.g., one or more touch inputs, one or more hardware control inputs, one or more button presses, one or more rotational inputs, one or more gesture inputs, and/or one or more spoken inputs) corresponding to selection of the continue check-in option; and in response to detecting the selection input corresponding to selection of the continue check-in option, the computer system maintains the workout-based check-in session without transmitting check-in notification information to the first external computer system. Automatically displaying the first user interface when the user notification criteria are satisfied allows for these operations to be performed without user input. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, the first user interface (e.g., 654) includes an end check-in option (e.g., 654b) that, when selected, causes the computer system to end the workout-based check-in session (or, in some embodiments, initiate a process for ending the workout-based check-in session). In some embodiments, while displaying the first user interface (e.g., 654), the computer system (e.g., 600) detects, via the one or more input devices, a selection input (e.g., 656b) (e.g., one or more user inputs) (e.g., one or more touch inputs, one or more hardware control inputs, one or more button presses, one or more rotational inputs, one or more gesture inputs, and/or one or more spoken inputs) corresponding to selection of the end check-in option. In response to detecting the selection input corresponding to selection of the end check-in option, the computer system causes the workout-based check-in session to be terminated; and transmits, to the first external computer system (e.g., 630), check-in termination information which causes the first external computer system to display an indication (e.g., 664) that the workout-based check-in session has been terminated. In some embodiments, when the workout-based check-in session is terminated, the computer system ceases automatic transmission of notifications pertaining to the computer system and/or the workout based on satisfaction of the notification criteria. Automatically displaying the first user interface when the user notification criteria are satisfied allows for these operations to be performed without user input. Additionally, displaying an option to end the workout-based check-in session allows for these operations to be performed with fewer user inputs. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, while the workout-based check-in session with the first external computer system is active: in accordance with a determination that the first user interface (e.g., 654) has been displayed for a threshold duration of time (e.g., one minute, two minutes, five minutes, ten minutes, or fifteen minutes) without user input interacting with the first user interface (e.g., without user input selecting option 654a) (e.g., the user of the computer system has not provided any input interacting with the first user interface and/or has not provided a particular type of input interacting with the first user interface), the computer system (e.g., 600) transmits, to the first external computer system (e.g., 630), first check-in notification information pertaining to the workout-based check-in session that causes the first external computer system to output first information (e.g., 668) (e.g., display information and/or output audio information) corresponding to the computer system, wherein the first check-in notification information includes information pertaining to a location (e.g., a geographic location) of the computer system and the first check-in notification information causes the first external computer system to display information pertaining to the location of the computer system (e.g., map 668d). In some embodiments, while the workout-based check-in session with the first external computer system is active: in accordance with a determination that the first user interface has not been displayed for the threshold duration of time (e.g., one minute, two minutes, five minutes, ten minutes, or fifteen minutes) without user input interacting with the first user interface, the computer system forgoes transmitting the first check-in notification information to the first external computer system. Automatically transmitting check-in information to the first external computer system when the user does not interact with the first user interface for a threshold duration of time allows for these operations to be performed without user input. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, while the workout-based check-in session with the first external computer system is active: in accordance with a determination that the first user interface has been displayed for the threshold duration of time without user input interacting with the first user interface (e.g., without user input selection option 654a), the computer system ceases display of the first user interface (e.g., 654) and displays via the one or more display generation components, a second user interface (e.g., 666) different from the first user interface, wherein the second user interface indicates that first check-in notification information has been transmitted to the first external computer system. In some embodiments, the second user interface indicates that the location of the computer system is being shared with the first external computer system. In some embodiments, while the workout-based check-in session with the first external computer system is active: in accordance with a determination that the first user interface has not been displayed for the threshold duration of time (e.g., one minute, two minutes, five minutes, ten minutes, or fifteen minutes) without user input interacting with the first user interface, the computer system forgoes displaying the second user interface and/or maintains display of the first user interface without displaying the second user interface. Automatically displaying the second user interface when the user does not interact with the first user interface for a threshold duration of time provides the user with visual feedback about a state of the system (e.g., the computer system has transmitted notification information to the first external computer system). Doing so also allows for these operations to be performed without user input. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, the second user interface (e.g., 666) includes a stop sharing option (e.g., 666c) that, when selected, causes the computer system to cease sharing location information pertaining to the location of the computer system with the first external computer system. In some embodiments, while displaying the second user interface, the computer system detects, via the one or more input devices, a selection input (e.g., one or more user inputs) (e.g., one or more touch inputs, one or more hardware control inputs, one or more button presses, one or more rotational inputs, one or more gesture inputs, and/or one or more spoken inputs) corresponding to selection of the stop sharing option; and in response to detecting the selection input corresponding to selection of the stop sharing option, the computer system ceases transmitting location information pertaining to the location of the computer system to the first external computer system. Automatically displaying the second user interface when the user does not interact with the first user interface for a threshold duration of time provides the user with visual feedback about a state of the system (e.g., the computer system has transmitted notification information to the first external computer system). Doing so also allows for these operations to be performed without user input. Additionally, displaying an option to stop sharing location information with the first external computer system allows for these operations to be performed with fewer user inputs, and also improves device security by allowing the user of the computer system to control if and/or when location information is shared with an external device. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

Note that details of the processes described above with respect to method 800 (e.g., FIG. 8) are also applicable in an analogous manner to the methods described above. For example, method 700 optionally includes one or more of the characteristics of the various methods described above with reference to method 800. For example, in some embodiments, the workout session recited in method 700 is the same workout session recited in method 800; and/or the workout-based check-in session recited in method 700 is the same workout-based check-in session recited in method 800. For brevity, these details are not repeated below.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

Although the disclosure and examples have been fully described with reference to the accompanying drawings, it is to be noted that various changes and modifications will become apparent to those skilled in the art. Such changes and modifications are to be understood as being included within the scope of the disclosure and examples as defined by the claims.

As described above, one aspect of the present technology is the gathering and use of data available from various sources to improve the delivery to users of check-in notifications or any other content that may be of interest to them. The present disclosure contemplates that in some instances, this gathered data may include personal information data that uniquely identifies or can be used to contact or locate a specific person. Such personal information data can include demographic data, location-based data, telephone numbers, email addresses, social network IDs, home addresses, data or records relating to a user's health or level of fitness (e.g., vital signs measurements, medication information, exercise information), date of birth, or any other identifying or personal information.

The present disclosure recognizes that the use of such personal information data, in the present technology, can be used to the benefit of users. For example, the personal information data can be used to deliver workout-based check-in notifications. Accordingly, use of such personal information data enables users to have calculated control of delivered notifications and/or content. Further, other uses for personal information data that benefit the user are also contemplated by the present disclosure. For instance, health and fitness data may be used to provide insights into a user's general wellness, or may be used as positive feedback to individuals using technology to pursue wellness goals.

The present disclosure contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. Such policies should be easily accessible by users, and should be updated as the collection and/or use of data changes. Personal information from users should be collected for legitimate and reasonable uses of the entity and not shared or sold outside of those legitimate uses. Further, such collection/sharing should occur after receiving the informed consent of the users. Additionally, such entities should consider taking any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices. In addition, policies and practices should be adapted for the particular types of personal information data being collected and/or accessed and adapted to applicable laws and standards, including jurisdiction-specific considerations. For instance, in the US, collection of or access to certain health data may be governed by federal and/or state laws, such as the Health Insurance Portability and Accountability Act (HIPAA); whereas health data in other countries may be subject to other regulations and policies and should be handled accordingly. Hence different privacy practices should be maintained for different personal data types in each country.

Despite the foregoing, the present disclosure also contemplates embodiments in which users selectively block the use of, or access to, personal information data. That is, the present disclosure contemplates that hardware and/or software elements can be provided to prevent or block access to such personal information data. For example, in the case of workout-based check-in notifications, the present technology can be configured to allow users to select to “opt in” or “opt out” of participation in the collection of personal information data during registration for services or anytime thereafter. In another example, users can select not to provide location information or other types of information for workout-based check-ins. In yet another example, users can select to limit the length of time user location data is maintained or entirely prohibit the transmission and/or sharing of user location data. In addition to providing “opt in” and “opt out” options, the present disclosure contemplates providing notifications relating to the access or use of personal information. For instance, a user may be notified upon downloading an app that their personal information data will be accessed and then reminded again just before personal information data is accessed by the app.

Moreover, it is the intent of the present disclosure that personal information data should be managed and handled in a way to minimize risks of unintentional or unauthorized access or use. Risk can be minimized by limiting the collection of data and deleting data once it is no longer needed. In addition, and when applicable, including in certain health related applications, data de-identification can be used to protect a user's privacy. De-identification may be facilitated, when appropriate, by removing specific identifiers (e.g., date of birth, etc.), controlling the amount or specificity of data stored (e.g., collecting location data a city level rather than at an address level), controlling how data is stored (e.g., aggregating data across users), and/or other methods.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data. For example, workout-based check-in notifications can be delivered without sharing location information or other types of personal information.

What is claimed is:

1. A computer system configured to communicate with one or more display generation components and one or more input devices, comprising:
one or more processors; and
memory storing one or more programs configured to be executed by the one or more processors, the one or more programs including instructions for:
detecting, via the one or more input devices, a first user input corresponding to a user request to initiate a workout session;
in response to detecting the first user input, initiating a workout session;
while the workout session is active, detecting, via the one or more input devices, one or more user inputs corresponding to a user request to activate a workout-based check-in session with an external computer system separate from the computer system, including selection of a displayed check-in option;
in response to detecting the one or more user inputs corresponding to the user request to activate the workout-based check-in session with an external computer system, initiating a process for activating a workout-based check-in session with a first external computer system separate from the computer system, wherein, while the workout-based check-in session is active, the computer system automatically transmits notifications to the first external computer system based on satisfaction of notification criteria pertaining to the computer system; and
while the workout-based check-in session with the first external computer system is active:
in accordance with a determination that notification criteria are satisfied, transmitting check-in notification information to the first external computer system, wherein the check-in notification information causes the first external computer system to output information corresponding to the computer system.

2. The computer system of claim 1, wherein initiating the process for activating the workout-based check-in session with the first external computer system comprises:
displaying, via the one or more display generation components, a contact selection user interface that includes a plurality of contact options for the workout-based check-in session, including:
a first contact option corresponding to a first contact that, when selected, causes the computer system to initiate a process for activating a workout-based check-in session with the first contact; and
a second contact option corresponding to a second contact different from the first contact that, when selected, causes the computer system to initiate a process for activating a workout-based check-in session with the second contact.

3. The computer system of claim 2, wherein the first contact option is displayed within a first region of the contact selection user interface based on a determination that the user of the computer system has previously activated a workout-based check-in session with the first contact.

4. The computer system of claim 1, the one or more programs further including instructions for:
while the workout session is active, concurrently displaying, via the one or more display generation components:
the check-in option; and
an end workout option that, when selected, causes the computer system to initiate a process for ending the workout session.

5. The computer system of claim 1, the one or more programs further including instructions for:
while the workout session is active, concurrently displaying, via the one or more display generation components:
the check-in option; and
a pause workout option that, when selected, causes the computer system to pause the workout session.

6. The computer system of claim 1, the one or more programs further including instructions for:
while the workout-based check-in session with the first external computer system is active, detecting that computer system movement criteria are satisfied, wherein the computer system movement criteria pertain to movement of the computer system; and
in response to detecting that the computer system movement criteria are satisfied, displaying a first alert, wherein:
the determination that notification criteria are satisfied includes a determination that the user has not interacted with the first alert within a threshold duration of time; and
the check-notification information is transmitted to the first external computer system in accordance with a determination that the user has not interacted with the first alert within the threshold duration of time.

7. The computer system of claim 1, the one or more programs further including instructions for:
while the workout-based check-in session with the first external computer system is active, detecting one or more user inputs corresponding to a user request to end the workout-based check-in session; and
in response to detecting the one or more user inputs corresponding to the user request to end the workout-based check-in session, ending the workout-based check-in session, wherein:
the determination that notification criteria are satisfied includes a determination that the user of the computer system has ended the workout-based check-in session; and
the check-notification information is transmitted to the first external computer system in accordance with a determination that the user of the computer system has ended the workout-based check-in session.

8. The computer system of claim 1, the one or more programs further including instructions for:
while the workout-based check-in session with the first external computer system is active, detecting one or more user inputs corresponding to a user request to end the workout session; and
in response to detecting the one or more user inputs corresponding to the user request to end the workout session, ending the workout session, wherein:
the determination that notification criteria are satisfied includes a determination that the workout session has ended; and
the check-notification information is transmitted to the first external computer system in accordance with a determination that workout session has ended.

9. The computer system of claim 1, the one or more programs further including instructions for:
while the workout-based check-in session with the first external computer system is active, detecting one or more user inputs corresponding to a user request to transmit an emergency call; and
in response to detecting the one or more user inputs corresponding to the user request to transmit an emergency call, transmitting an emergency call, wherein:
the determination that notification criteria are satisfied includes a determination that the user of the computer system has initiated an emergency call; and
the check-notification information is transmitted to the first external computer system in accordance with a determination that the user of the computer system has initiated an emergency call.

10. The computer system of claim 1, the one or more programs further including instructions for:
while the workout-based check-in session with the first external computer system is active, detecting that a connected device that was in communication with the computer system is no longer in communication with the computer system, wherein:
the determination that notification criteria are satisfied includes a determination that the connected device is no longer in communication with the computer system; and
the check-notification information is transmitted to the first external computer system in accordance with a determination that the connected device is no longer in communication with the computer system.

11. The computer system of claim 1, the one or more programs further including instructions for:
while the workout session is active, displaying, via the one or more display generation components, a workout session user interface, wherein the workout session user interface displays one or more physical activity metrics that correspond to the workout session, and displaying the workout session user interface includes:
in accordance with a determination that a workout-based check-in session is active, displaying a check-in indication that is indicative of an active workout-based check-in session; and
in accordance with a determination that workout-based check-in session is not active, forgoing display of the check-in indication.

12. The computer system of claim 1, the one or more programs further including instructions for:
while the workout-based check-in session with the first external computer system is active, detecting that user notification criteria are satisfied; and
in response to detecting that the user notification criteria are satisfied, displaying, via the one or more display generation components, a first user interface that pertains to the workout-based check-in session.

13. The computer system of claim 12, wherein the first user interface includes a continue check-in option that, when selected, causes the computer system to maintain the workout-based check-in session without transmitting check-in notification information to the first external computer system.

14. The computer system of claim 12, wherein:
the first user interface includes an end check-in option that, when selected, causes the computer system to end the workout-based check-in session; and
the method further comprises:
while displaying the first user interface, detecting, via the one or more input devices, a selection input corresponding to selection of the end check-in option; and
in response to detecting the selection input corresponding to selection of the end check-in option:
causing the workout-based check-in session to be terminated; and
transmitting, to the first external computer system, check-in termination information which causes the first external computer system to display an indication that the workout-based check-in session has been terminated.

15. The computer system of claim 12, the one or more programs further including instructions for:
while the workout-based check-in session with the first external computer system is active:
in accordance with a determination that the first user interface has been displayed for a threshold duration of time without user input interacting with the first user interface, transmitting, to the first external computer system, first check-in notification information pertaining to the workout-based check-in session that causes the first external computer system to output first information corresponding to the computer system, wherein the first check-in notification information includes information pertaining to a location of the computer system and the first check-in notification information causes the first external computer system to display information pertaining to the location of the computer system.

16. The computer system of claim 15, the one or more programs further including instructions for:
while the workout-based check-in session with the first external computer system is active:
in accordance with a determination that the first user interface has been displayed for the threshold duration of time without user input interacting with the first user interface:
ceasing display of the first user interface; and
displaying, via the one or more display generation components, a second user interface different from the first user interface, wherein the second user interface indicates that first check-in notification information has been transmitted to the first external computer system.

17. The computer system of claim 16, wherein the second user interface includes a stop sharing option that, when selected, causes the computer system to cease sharing location information pertaining to the location of the computer system with the first external computer system.

18. The computer system of claim 11, the one or more programs further including instructions for:
while the workout-based check-in session is active, and while displaying the workout session user interface, detecting that the computer system has transitioned from a first power mode to a low power mode, wherein, in the low power mode, the computer system activates one or more power saving measures that are not activated in the first power mode; and
in response to detecting that the computer system has transitioned from the first power mode to the low power mode, updating the check-in indication to indicate that the computer system is in the low power mode.

19. The computer system of claim 18, the one or more programs further including instructions for:
in response to detecting that the computer system has transitioned from the first power mode to the low power mode, transmitting low power information to the first external computer system that causes the first external computer system to display an indication that the computer system is in the low power mode.

20. The computer system of claim 1, the one or more programs further including instructions for:
displaying, via the one or more display generation components, a messaging session user interface that corresponds to a messaging session that includes the user of the computer system and a first remote user that is associated with the first external computer system;
while displaying the messaging session user interface, detecting, via the one or more display generation components, a first set of user inputs; and
in response to detecting the first set of user inputs:
in accordance with a determination that a workout session is active when the first set of user inputs is detected, displaying, via the one or more display generation components, a selectable user interface object that, when selected, causes the computer system to initiate a process for activating a workout-based check-in session with a respective external computer system separate from the computer system; and
in accordance with a determination that a workout session is not active when the first set of user inputs is detected, forgoing display of the selectable user interface object.

21. A non-transitory computer-readable storage medium storing one or more programs configured to be executed by one or more processors of a computer system that is in communication with one or more display generation components and one or more input devices, the one or more programs including instructions for:
detecting, via the one or more input devices, a first user input corresponding to a user request to initiate a workout session;
in response to detecting the first user input, initiating a workout session;
while the workout session is active, detecting, via the one or more input devices, one or more user inputs corresponding to a user request to activate a workout-based check-in session with an external computer system separate from the computer system, including selection of a displayed check-in option;
in response to detecting the one or more user inputs corresponding to the user request to activate the workout-based check-in session with an external computer system, initiating a process for activating a workout-based check-in session with a first external computer system separate from the computer system, wherein, while the workout-based check-in session is active, the computer system automatically transmits notifications to the first external computer system based on satisfaction of notification criteria pertaining to the computer system; and
while the workout-based check-in session with the first external computer system is active:
in accordance with a determination that notification criteria are satisfied, transmitting check-in notification information to the first external computer system, wherein the check-in notification information causes the first external computer system to output information corresponding to the computer system.

22. A method, comprising:
at a computer system that is in communication with one or more display generation components and one or more input devices:
detecting, via the one or more input devices, a first user input corresponding to a user request to initiate a workout session;
in response to detecting the first user input, initiating a workout session;
while the workout session is active, detecting, via the one or more input devices, one or more user inputs corresponding to a user request to activate a workout-based check-in session with an external computer system separate from the computer system, including selection of a displayed check-in option;
in response to detecting the one or more user inputs corresponding to the user request to activate the workout-based check-in session with an external computer system, initiating a process for activating a workout-based check-in session with a first external computer system separate from the computer system, wherein, while the workout-based check-in session is active, the computer system automatically transmits notifications to the first external computer system based on satisfaction of notification criteria pertaining to the computer system; and
while the workout-based check-in session with the first external computer system is active:
in accordance with a determination that notification criteria are satisfied, transmitting check-in notification information to the first external computer system, wherein the check-in notification information causes the first external computer system to output information corresponding to the computer system.
